(19) United States
(12) Patent Application Publication
(10) Pub. No.: US 2025/0236820 A1
(43) Pub. Date: Jul. 24, 2025

(54) BIODEGRADABLE MICROCAPSULES WITH IMPROVED STORAGE STABILITY, PROCESS FOR PREPARING THE SAME AND METHOD OF USE THEREOF

(71) Applicant: ISP Investments LLC, Wilmington, DE (US)

(72) Inventors: Karen MITCHINSON, Baildon (GB); Abdul WAHAB HUSSAIN, Thornton (GB); Alan FERNYHOUGH, Idle (GB); OSAMA M. MUSA, Bedminster, NJ (US)

(73) Assignee: ISP Investments LLC, Wilmington, DE (US)

(21) Appl. No.: 18/729,490

(22) PCT Filed: Jan. 17, 2023
(86) PCT No.: PCT/US2023/010942

§ 371 Date: Jul. 16, 2024

Related U.S. Application Data
(60) Provisional application No. 63/300,204, filed on Jan. 17, 2022.

(51) Int. Cl. C11D 7/50; B01J 31/02; C11D 3/50; C11D 7/32; C11D 17/00

(52) U.S. Cl. CPC C11D 7/50; B01J 31/0237; C11D 3/505; C11D 7/3254; C11D 17/0039; C11D 2111/12

(57) ABSTRACT

The present application provides biodegradable microcapsules, based on specific poly ß-amino ester shells that can encapsulate and retain cargoes such as, lipophilic, or hydrophobic core materials comprising fragrances, butters, essential or other oils; or oil solubilized ingredients, process of making said biodegradable microcapsules and their applications in various industries. Present application further provides biodegradable shell materials that show evidence of biodegradation or non-persistence in aquatic based and/or soil or compost based environments, and which are stable on storage before use.

### FIELD OF THE INVENTION

The present invention relates to biodegradable microcapsules, that can encapsulate and retain cargoes such as, lipophilic, or hydrophobic core materials comprising fragrances, butters, essential or other oils; or oil solubilized ingredients, process of making said biodegradable microcapsules and their applications in various industries. Present invention further relates to biodegradable shell materials that show evidence of biodegradation or non-persistence in aquatic based and/or soil or compost based environments and yet which can show some storage stability in aqueous media, or formulated end products.

### BACKGROUND OF THE INVENTION

Conventionally, microcapsules: (a) provide protection and stability to actives or ingredients entrapped inside the microcapsule; (b) facilitate, trigger, or control release of the entrapped actives or ingredients, (c) extend the life of the actives, (d) reduce the threat of exposures or (e) enable easily handling of entrapped actives which are otherwise toxic in nature or difficult to handle.

The microcapsules of the invention have an inner core material comprising lipophilic/hydrophobic compounds surrounded by an outer polymeric shell. The release rate of the core material and the diffusion of the core material through the capsule wall can often be controlled by varying the wall composition and/or the degree of crosslinking of the wall (shell) material. Further, the degree of crosslinking of the wall material directly impacts the strength and nature of the microcapsule wall.

A highly beneficial use, for example, of microcapsules is for the prolongation of fragrances, essential oils, or other lipophilic or oil solubilized ingredients which have been encapsulated inside a polymer shell. Typically, the technologies or materials used for encapsulation of fragrances or similar molecules (cargoes) have included melamine formaldehyde, urea-formaldehyde, or poly-urea/urethane technologies or acrylate technologies, most often using classical interfacial polymerizations. Because such cargoes are often aggressively solvating or plasticizing for many polymers and/or are volatile or have low boiling components difficult to retain within polymeric shells, such polymer shell walls used for their encapsulation are crosslinked networks of such polymers for stability and durability in the formulations in which they are used, for example, laundry/washing products, household cleaning products, hair care products skin care products among others. They are not designed to be biodegradable or non-persistent in the environments they may end up in. There are some known examples of biodegradable polymers used in microcapsule shell walls. They include polyesters or poly-ß-amino-esters, for example. However, in most cases, they are typically not encapsulating fragrances or essential oils or other solvating or plasticizing lipophilic or oil solubilized components for use in such consumer products, and/or are not biodegradable in relevant end media and/or are not stable on storage in aqueous or formulated end products that may contain aggressive surfactants or other components, and which may be formulated at pH's significantly above or below neutral.

U.S. Pat. No. 8,287,849 (assigned to Massachusetts Institute of Technology) and scientific publication—“Degradable Poly-ß-amino esters: Synthesis, Characterization, and Self-Assembly with Plasmid DNA” (written by Lynn, D. M, Langer, R.) published in J. Am. Chem. Soc. 2000, 122, 44, 10761-10768 disclose that poly-ß-amino esters are typically unstable in solutions with wide pH range over hours to days, and are biodegradable in physiological or biomedical environments, comprising a compound prepared by reacting primary amine with bis-(acrylate ester).

U.S. Pat. No. 8,557,231 (assigned to Massachusetts Institute of Technology) describes poly-ß-amino esters which are synthesized in organic solvents such as tetrahydrofuran (THF) or dichloromethane (DCM) and after isolation are then suggested as being useful in a complex double emulsion encapsulation process, typically also using a solvent (later to be removed) such as DCM, to make capsules for delivery of drugs or pharma based actives, for near term controlled release in biomedical physiological environments.

U.S. Pat. No. 8,945,622 B2 (assigned to Council of Scientific and Industrial Research CSIR) discloses a sustained release composition, useful for delivering active pharmaceutical agents comprising a graft polymer with a polyester backbone having the formula P [A(x)B(y)C(z)] prepared from diol (A), a dicarboxylic acid or acid anhydride (B) and a monomer (C) with pendant unsaturation onto which is grafted a polyacrylic or methacrylic acid chain. This patent describes tablet making processes.

US Patent Publication US 2003/0224060 (assigned to L'Oreal) discloses nanocapsules having specific targeted cargo of retinoyl esters, which is described as a lipophilic active agent, and which have a water-insoluble envelope, comprising at least one polyester polyol wherein, this pre-made polyester polyol has been obtained by polycondensation of an aliphatic dicarboxylic acid or derivative with at least two alkane diols or with at least one alkane diol and at least one hydroxyalkyl alkane diol.

U.S. Patent Publication US 2007/0009441 (assigned to Molecular Therapeutics Inc.) discloses nanoparticle synthesis, their use in nanoscale (typically below 200 nm) encapsulations of water-soluble drugs or water-insoluble actives as solids for pharmaceutical applications. Biodegradability/biocompatibility in simulated physiological media was shown and in one aspect an itaconate polyester was used with specifically added crosslinkers for radical crosslinking, via aqueous radical initiator systems, with the itaconate polymer.

U.S. Patent Publication US 2020/164332 (assigned to Calyxia SAS) describes a complex multi-layered microcapsule in which one polymer shell may contain esters, but which is made by a very complicated double emulsion process, and which uses radically polymerized monomer or polymers with added crosslinker.

European Patent Application EP 0517669 A1 (assigned to Sandoz) discloses process for microencapsulation of agrochemicals, obtained by microencapsulating an agrochemical in a crosslinked polymer capsule which is in part a polyester polymer, wherein such a process comprises the steps of (a) dissolving or suspending the agrochemical in a non-aqueous liquid mixture comprising unsaturated polyester resin and a vinyl monomer (preferentially styrene), (b) emulsifying said solution or suspension in water to a desired particle size; and (c) effecting crosslinking of the unsaturated polyester resin and vinyl monomer to produce the microcapsules.

PCT Publication WO 2017125395 (assigned to BASF SE) discloses ‘biodegradable’ (in soil) polyester capsules comprising an aqueous core and a pesticide, wherein the capsule shell comprises a polyester, and the capsule core comprises a water-soluble pesticide (so a hydrophilic core), and at least 10 wt. % of water based on the total weight of the capsule core. Further, acid chlorides are used for its practical application to enable moderate temperatures and short reaction times for formation of the in-situ polyester in the presence of the cargo.

US 2020/0360889 (assigned to Gemminov) describes a method to prepare biodegradable microcapsules with lipophilic cores wherein the shell material is based on poly-ß-amino-esters. The method described is an interfacial oil-in-water polymerization process wherein one reactant (an amine; donor) is added, at a significant excess of molar equivalents (of reactive functional groups), to a pre-made oil-in-water emulsion containing the other reactant (an acrylate, acceptor). Secondary coatings based on polymers are optionally applied at the end of the interfacial polymerization as a water solution.

The Scientific publication—“Fragrance-containing microcapsules based on interfacial thiol-ene polymerization” (by Liao et al) published in J. Appl. Polym. Sci. 2016, 133,43905 doi: 10.1002/App.43905, discloses fragrance capsules with a poly-ß-thio ester shell wall, made using a classical interfacial polymerization route wherein a water-soluble reactant is added in a water solution to a pre-made emulsion of an oil phase (containing the other reactant) and a water/stabilizer phase.

Such prior arts have numerous disadvantages wherein: (i) some are for water soluble actives and so not suitable for hydrophobic or lipophilic materials; (ii) few show or claim or are designed for biodegradability, none show or claim biodegradability in ambient aquatic environments or in related OECD tests, (iii) many use organic solvents to enable encapsulation which are problematic in removal and for use with volatile cargoes, (iv) some make the polymer shell wall itself in the presence of the cargo via interfacial polymerizations where one reactive or catalytic ingredient is either in the water phase or is added separately to a pre-made oil-in-water emulsion comprising the other reactant, which is in the oil phase and where, as such, necessarily require a large excess of a the added reactant (e.g. amine), and/or require the use of undesirable solvents and their evaporation, and (v) few, if any, show any ability to be stable on storage as made with fragrance or oils or other plasticizing cargoes inside, or show stability or utility after storage in end product formulations as are used in home or personal care applications, which may have pH extremes or surfactants or salts or solvents or other additives that may plasticize or attack the shell wall.

There remains a significant challenge to encapsulate lipophilic or hydrophobic cargoes in a polymeric shell which can biodegrade in aquatic or other media and which is robust enough to hold the cargo (often in aqueous based formulations of personal care or household or other products) until release is triggered or required and/or to release it in a gradual process or controlled way.

Current inventors aim to meet these criteria and so enable production of microcapsules that have a shell material that is biodegradable or non-persistent, particularly in aquatic media/waterways, and yet which can retain a hydrophobic or lipophilic cargo or a volatile or a plasticizing or oil solubilized cargo such as a fragrance or an essential oil or other oil, and which is stable on storage in a product form until use.

### SUMMARY OF THE INVENTION

We have surprisingly discovered that volatile or plasticizing hydrophobic or lipophilic ingredients such as fragrances, oils and other lipophilic cargoes can be encapsulated within robust storage stable polymer shells incorporating specific ß-amino-ester moieties in their backbones and/or in their branches and/or in their crosslinks and, furthermore, in addition, that through the associated polymer shell precursors and polymer architectures, such as linear, branched, or crosslinked polymers, such polymer shell systems can meet important biodegradability criteria and in particular such criteria for biodegradability or non-persistence in ambient aquatic environments such as seawater, river/surface water, effluents, and/or other water treatment process streams (e.g. activated sludge).

In one aspect, the present application provides a microcapsule comprising: (i) a polymeric microcapsule shell; and (ii) a lipophilic core; wherein, the polymeric microcapsule shell comprises a poly-ß-amino-ester polymer, co-polymer of poly-ß-amino-ester, terpolymer of poly-ß-amino-ester, a crosslinked polymer of poly-ß-amino-ester, or mixtures thereof; wherein, the microcapsule is storage stable, and its polymeric shell is biodegradable.

In another aspect, the present application provides a method for preparing a microcapsule, the method comprising: (a) preparing an oil-in-water emulsion of (i) an oil phase comprising at least one multifunctional amine donor with at least one multifunctional acceptor, and at least one lipophilic core; and (ii) a water phase comprising at least one stabilizer, at least one defoamer, or at least one emulsifier, (b) optionally adding at least one catalyst, at least one diluent or at least one initiator to the oil phase; (c) heating the oil-in-water emulsion with stirring to a temperature between 25° C. and 100° C. and so forming the polymeric microcapsule shell by an in-situ oil-in-water reaction of the amine donor(s) with the acceptor component(s); and (d) obtaining the lipophilic core encapsulated in a polymeric microcapsule shell.

In another aspect, the present application provides method for preparing a microcapsule, the method comprising: (a) pre-reacting at least one multifunctional amine donor with a modifying reagent to form a modified amine donor; (b) preparing an oil-in-water emulsion of (i) an oil phase comprising at least one amine functional donor including at least one modified amine donor as prepared in (a), with at least one acceptor(s), and at least one lipophilic core; and (ii) a water phase comprising at least one stabilizer, at least one defoamer or at least one emulsifier, (c) optionally adding at least one catalyst, at least one diluent or at least one initiator to the oil phase; (d) heating the oil-in-water emulsion with stirring to a temperature between 25° C. and 100° C. to form the polymeric microcapsule shell by an in-situ oil-in-water reaction of the amine donor(s) with the acceptor component(s); and (e) obtaining the lipophilic core encapsulated in a polymeric microcapsule shell.

In yet another aspect, the present application provides method for preparing a microcapsule, the method comprising: (a) preparing an oil-in-water emulsion of (i) an oil phase comprising at least one amine functional donor with at least one acceptor, and at least one lipophilic core; and (ii) a water phase comprising at least one stabilizer, at least one defoamer or at least one emulsifier, (b) optionally adding at least one catalyst, at least one diluent or at least one initiator to the oil phase; (c) heating the oil-in-water emulsion with stirring to a temperature between 25° C. and 100° C. and so forming the polymeric microcapsule shell by an in-situ oil-in-water reaction of the amine donor(s) with the acceptor component(s); and (e) obtaining the lipophilic core encapsulated in a polymeric microcapsule shell, and (f) effecting a post-modification reaction on the polymeric shell material to add hydrophobic, sterically hindered, charged, rigid or pH responsive functional groups to the shell material.

### BRIEF DESCRIPTION OF THE DRAWINGS

Further embodiments of the present application can be understood with the appended figures.

FIG. 1 illustrates Optical Micrograph Images of Microcapsules of Example 1 (220-31-2). Microcapsules of Example 1 (220-31-2) poly-ß-amino ester capsules prepared via in situ polymerization process using multifunctional amine: images of capsules before and after crushing under a microscope cover-slip.

FIG. 2 illustrates Optical Micrograph Images of Microcapsules of Example 2 (220-61-1). Microcapsules of Example 2 (220-61-1)—incorporating mono-functional hydrophobic methacrylate (LMA) pre-modified PEHA: capsules before and after crushing under a microscope cover-slip.

FIG. 3 illustrates Optical Micrograph Images of Microcapsules of Example 3 (220-62-1). Microcapsules of Example 3 (220-62-1)—incorporating mono-functional charged methacrylate (MBJ) pre-modified PEHA: capsules before and after crushing under a microscope cover-slip.

FIG. 4 illustrates Optical Micrograph Images of Microcapsules of Example 2 & 3 (‘pre-reactions’) capsules before (upper image in series below) and after crushing (lower image in series below) under a microscope cover-slip (220-40-2; 220-42-2; 220-43-2).

FIG. 5 illustrates Optical Micrograph Images of Microcapsules made by mono-acrylate modified amine (PEHA) and then reacted with Di-PETA. Images before (upper image) and after (lower image) crushing under a microscope slide (220-44-1; 220-45-2; 220-46-1).

FIG. 6 illustrates Optical Micrograph Images of Microcapsules mono-acrylate modified amine (PEHA) and then reacted with Di-PETA. Images before (upper image) and after (lower image) under a microscope slide (220-58-1; 222-05-1; 222-06-1).

FIG. 7 illustrates Optical Micrograph Images of Microcapsules made from Carboxy ethyl acrylate (CEA) pre-modified PEHA 222-07-1) and Acrylic acid (AA) pre-modified PEHA (222-08-1).

FIG. 8 illustrates Optical Micrograph Images of Microcapsules of the invention made by mono-acrylate (or acrylamide) pre-modified amine (PEHA) and then reacted with Di-PETA. Images before (upper image) and after (lower image) crushing under a microscope slide (220-60-1; 220-61-1).

FIG. 9 illustrates Optical Micrograph Images of Microcapsules of the invention made by mono-methacrylate pre-modified amine (PEHA) and then reacted with Di-PETA. Images before (upper image) and after (lower image) crushing under a microscope slide (220-63-1; 220-58-1).

FIG. 10 illustrates Optical Micrograph Images of Microcapsules of Example 4 (220-73-1) incorporating a hydrophobic methacrylate (LMA) in a post shell formation reaction. Images of capsules before and after crushing under a microscope cover-slip.

FIG. 11 illustrates Optical Micrograph Images of other microcapsules prepared by processes analogous to Example 4 (‘post modification reactions’)—images of capsules before (upper image) and after crushing (lower image).

FIG. 12 illustrates Optical Micrograph Images of microcapsules prepared from AMPS post-modified PEHA (220-92-1) and CEAO post-modified PEHA (220-92-2).

FIG. 13 illustrates Optical Micrograph Images of microcapsules prepared from CEA post-modified PEHA (220-92-3) and AA post-modified PEHA (220-92-4).

FIG. 14 illustrates Optical Micrograph Images of microcapsules of the invention made Capsules from Example 6 made from Di-PETA with PEHA with secondary coating (221-3-1). Images before (left image) and after (right image) crushing under a microscope slide.

FIG. 15 illustrates Optical Micrograph Images of microcapsules of the invention Capsules from Example 7 made from Di-PETA with MBJ modified PEHA and secondary coating under a microscope slide (221-10-1).

FIG. 16 illustrates Optical Micrograph Images of microcapsules of the invention Capsules from 221-11-1 made from Di-PETA with MBS modified PEHA and secondary coating under a microscope slide.

FIG. 17 illustrates Optical Micrograph Images of microcapsules—(221-13-1) of the invention Capsules from Examples with secondary coatings (as made) under a microscope slide.

FIG. 18 illustrates Optical Micrograph Images of microcapsules of the invention Capsules from 221-20-1 from MBJ pre-modified PEHA with DiPETA and secondary coating with higher CCG and pH=10.5 adjusted to 4.58 (crosslinked with glutaraldehyde)—(with secondary coating, also including zein protein): before (left) and after crushing under a microscope slide.

FIG. 19 illustrates Optical Micrograph Images of microcapsules of the invention Capsules from 221-21-1 (with secondary coating and with added zein, higher CCG and pH=8.8 (crosslinked with glutaraldehyde): before (left) and after crushing under a microscope slide.

FIG. 20 illustrates Optical Micrograph Images of microcapsules of the invention Capsules made with selected amine donor-acceptor combinations (no pre- or post-modifications). Capsules from Example 1 made from PEHA and Di-PETA with added mono-acceptors mixed in with Di-PETA from the start: tert-butyl acrylate (t-BA; 222-13-1) and trimethylamine ethyl acrylate chloride (MBS; 222-14-1).

FIG. 21 illustrates Optical Micrograph Images of microcapsules of the invention, Microcapsules from 230-11-1 (PBAE reactively linked with hydrophobic polysaccharide—Example 10).

FIG. 22 illustrates Optical Micrograph Images of microcapsules of the invention, microcapsules from 230-20-1 (PBAE copolymer (co-ß-thio-ester) reactively linked with hydrophobic polysaccharide—Example 11).

FIG. 23 illustrates Optical Micrograph Images of microcapsules of the invention, microcapsules from 229-37-1 (PBAE copolymer (co-ß-thio-ester) with spray dried outer coating containing hydrophobic polysaccharide (OSA starch) Example 12).

FIG. 24 illustrates Optical Micrograph Images of microcapsules of the invention, microcapsules from 226-24-3 (PBAE with hydrophobic polysaccharide and tannic acid crosslinking-overcoating; Example 13).

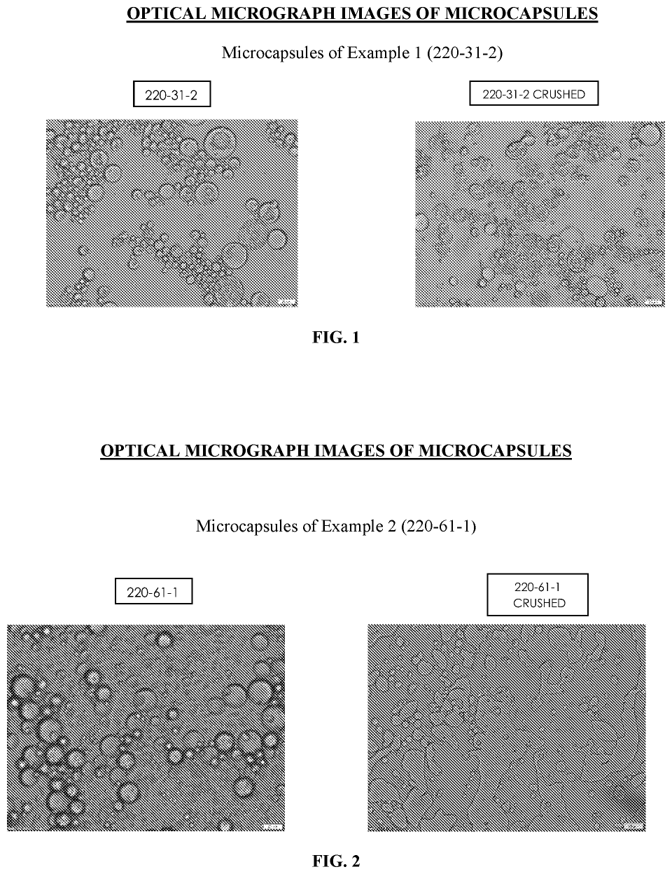

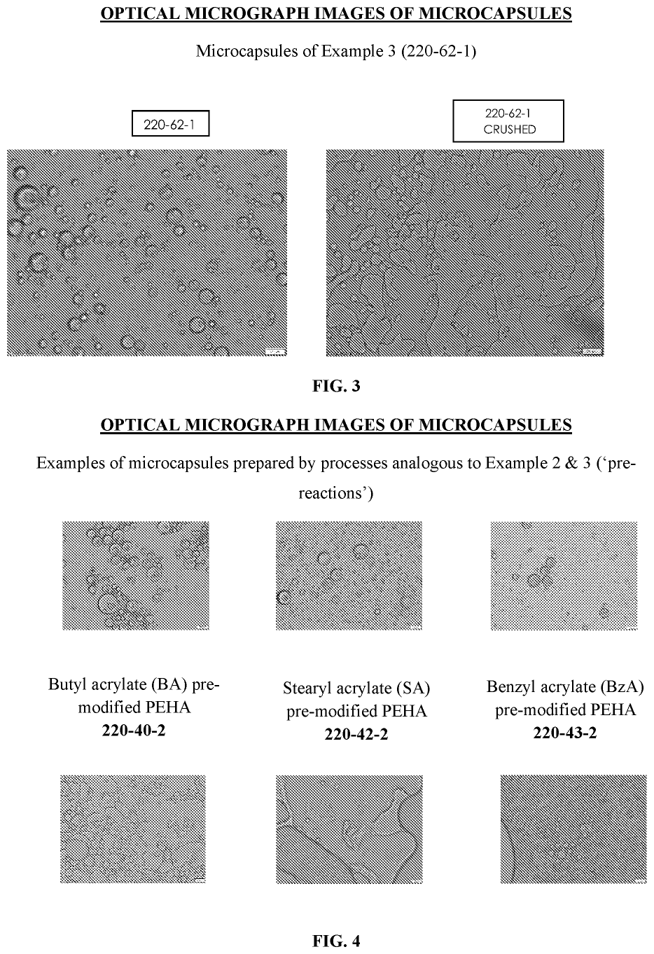

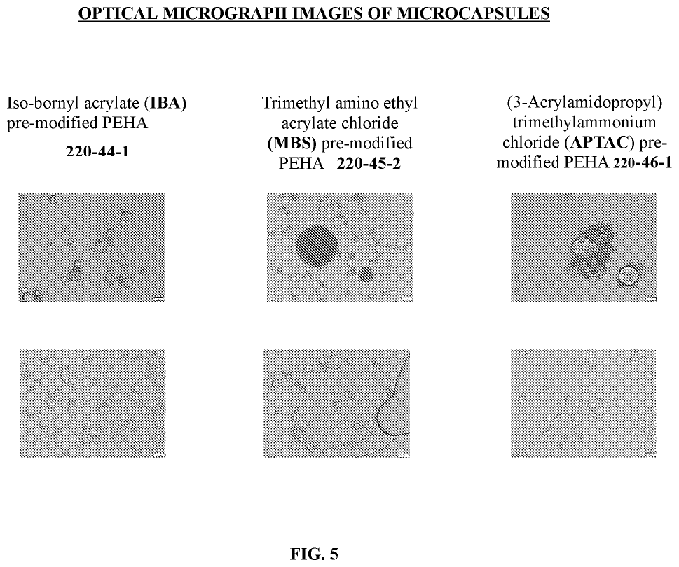

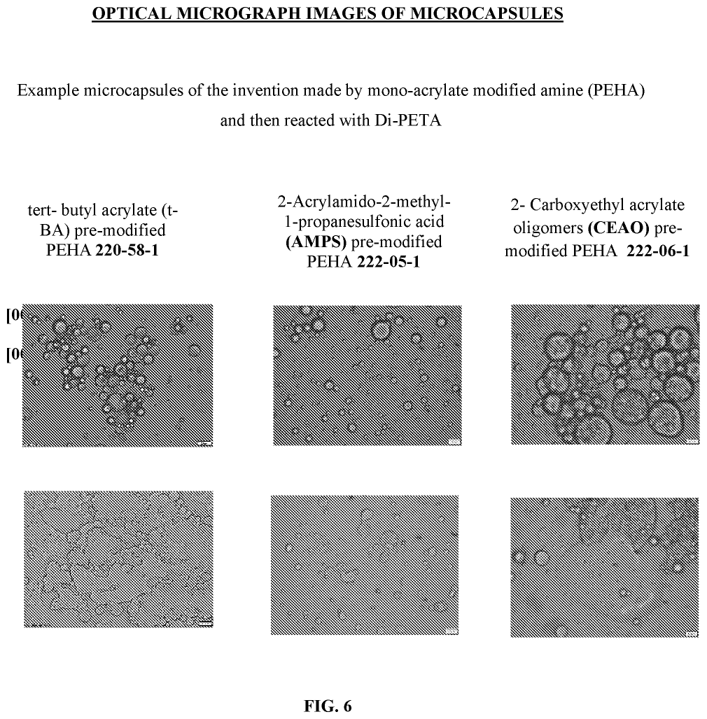

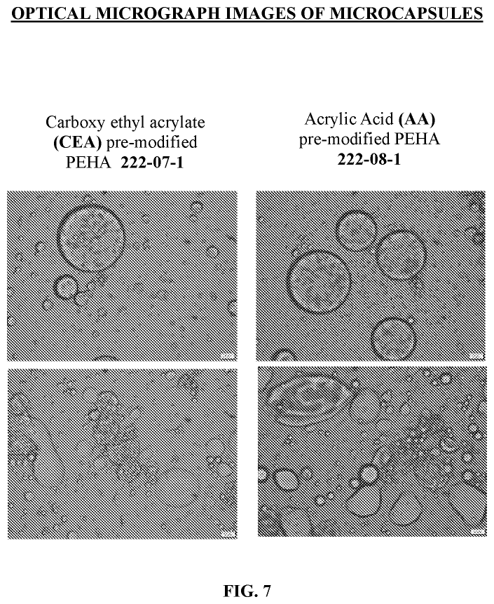

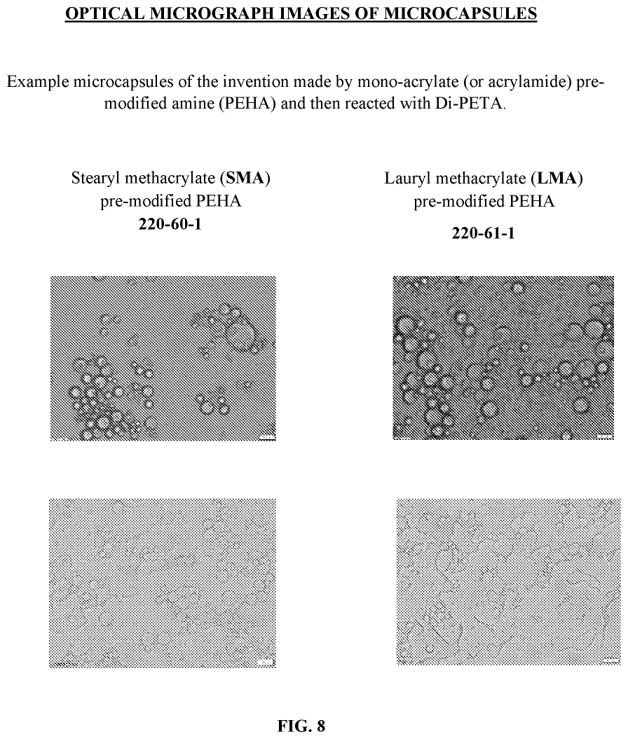

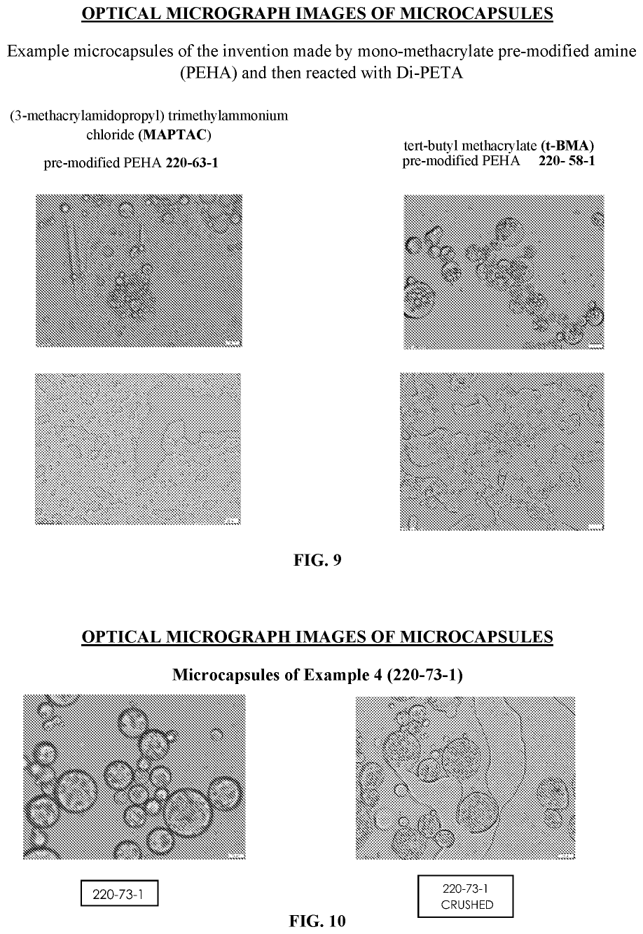

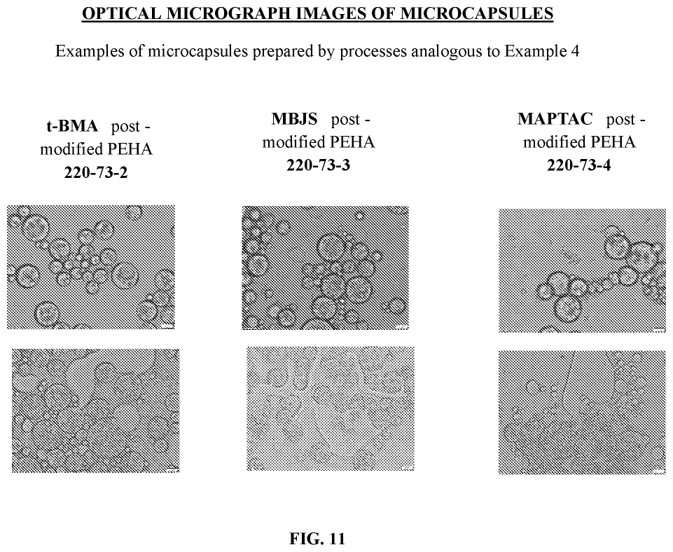

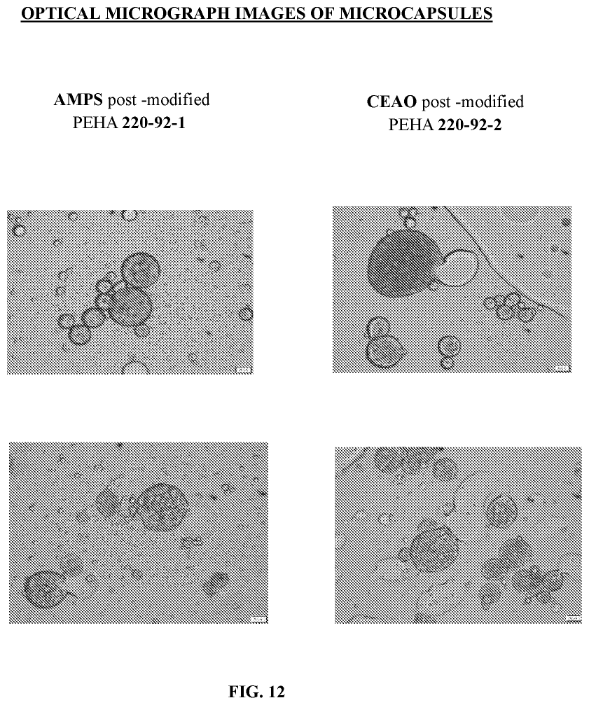

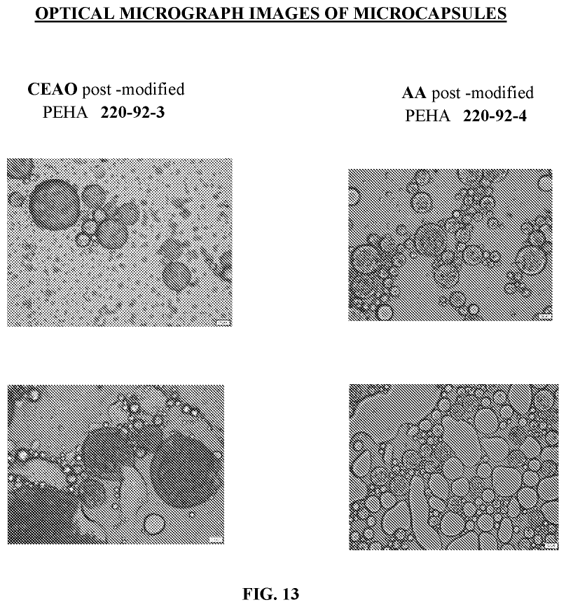

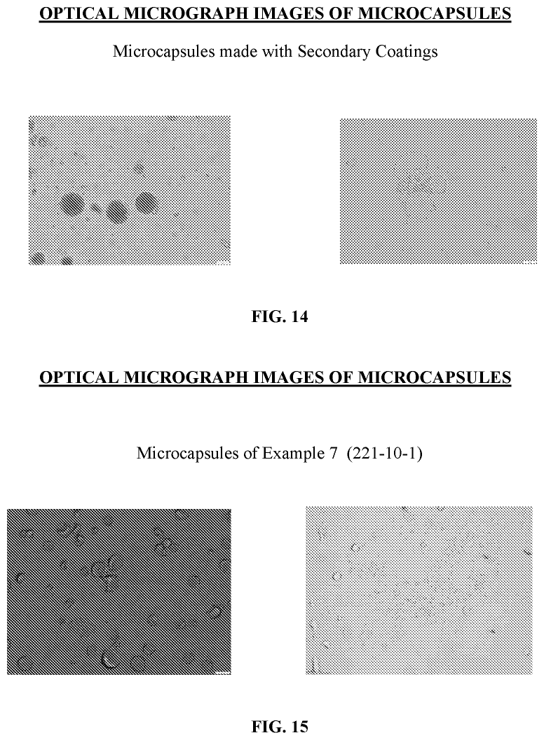

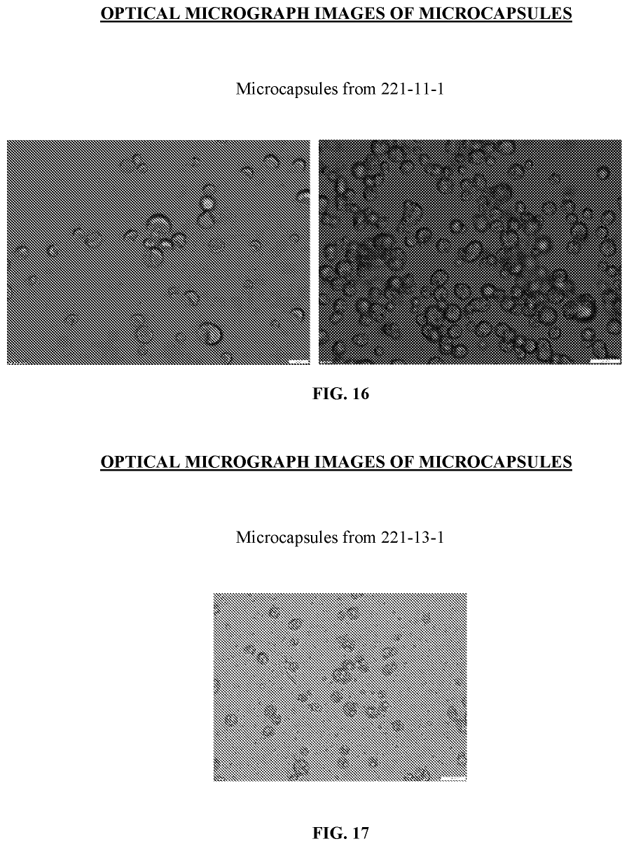

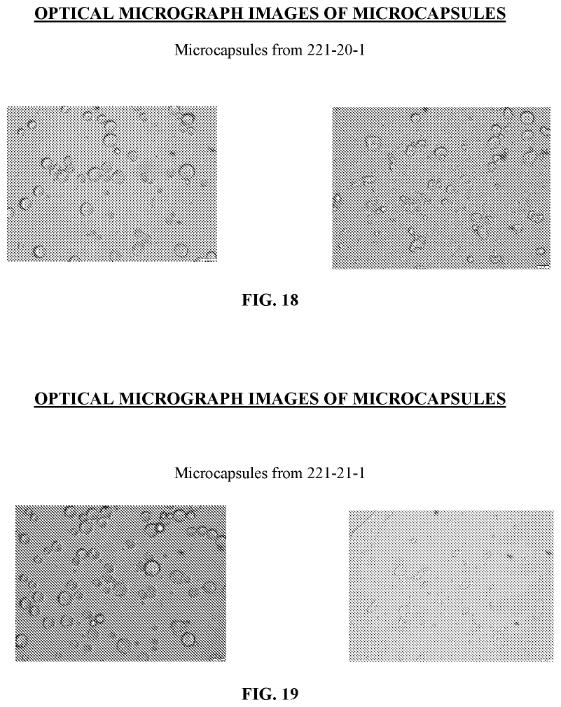

### DETAILED DESCRIPTION OF THE INVENTION

The present invention is directed to a biodegradable microcapsule, based on specific poly-ß-amino ester shells that can encapsulate and retain cargoes such as, lipophilic, or hydrophobic core materials comprising fragrances, butters, essential or other oils; or oil solubilized ingredients; process of making said biodegradable microcapsules and their applications in various industries. For physical-mechanical encapsulation methods, such as spray drying, particle size control is generally achieved through control of the physical conditions under which the involved processes are carried out. Before explaining at least one aspect of the disclosed and/or claimed inventive concept(s) in detail, it is to be understood that the disclosed and/or claimed inventive concept(s) is not limited in its application to the details of construction and the arrangement of the components or steps or methodologies set forth in the following description or illustrated in the drawings. The disclosed and/or claimed inventive concept(s) is capable of other aspects or of being practiced or carried out in various ways. Also, it is to be understood that the phraseology and terminology employed herein is for the purpose of description and should not be regarded as limiting.

As utilized in accordance with the disclosure, the following terms, unless otherwise indicated, shall be understood to have the following meanings.

Unless otherwise defined herein, technical terms used in connection with the disclosed and/or claimed inventive concept(s) shall have the meanings that are commonly understood by those of ordinary skill in the art. Further, unless otherwise required by context, singular terms shall include pluralities and plural terms shall include the singular.

The singular forms “a,” “an,” and “the” include plural forms unless the context clearly dictates otherwise specified or clearly implied to the contrary by the context in which the reference is made. The term “Comprising” and “Comprises of” includes the more restrictive claims such as “Consisting essentially of” and “Consisting of”.

For purposes of the following detailed description, other than in any operating examples, or where otherwise indicated, numbers that express, for example, quantities of ingredients used in the specification and claims are to be understood as being modified in all instances by the term “about”. The numerical parameters set forth in the specification and attached claims are approximations that may vary depending upon the desired properties to be obtained in carrying out the invention.

All percentages, parts, proportions, and ratios as used herein, are by weight of the total composition, unless otherwise specified. All such weights as they pertain to listed ingredients are based on the active level and, therefore; do not include solvents or by-products that may be included in commercially available materials, unless otherwise specified.

All publications, articles, papers, patents, patent publications, and other references cited herein are hereby incorporated herein in their entirety for all purposes to the extent consistent with the disclosure herein.

The use of the term “at least one” will be understood to include one as well as any quantity more than one, including but not limited to, 1, 2, 3, 4, 5, 10, 15, 20, 30, 40, 50, 100, etc. The term “at least one” may extend up to 100 or 1000 or more depending on the term to which it is attached. In addition, the quantities of 100/1000 are not to be considered limiting as lower or higher limits may also produce satisfactory results.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “core” and “cargo” as used throughout the specification are inclusive and refer to same ingredient forming part of the microcapsule encapsulation.

The term “each independently selected from the group consisting of” means when a group appears more than once in a structure, that group may be selected independently each time it appears.

The term “hydrophobic” as used herein refers to relatively water repelling, and hydrophobic groups are groups which inhibit the access of water molecules to the ß-amino ester bond environment.

The term “sterically hindering groups” as used herein refers to groups which ‘crowd out’ or shield the ß-amino ester bond environment so that the ß-amino-ester bonds are partially shielded by neighboring groups and so water or larger molecules cannot easily approach and so react with the ß-amino ester bonds.

The term “charged” as used herein refers to bearing a positive or a negative charge, as is well known for certain classes of molecules in organic chemistry.

The term “pH Responsive”, or “pH stabilizing”, or “pH sensitive” (pH responsive is used to cover all such terms or implied variations) as used herein refers to a molecule or group that can change its structure or shape in way to be stable in a pH extreme way from neutral and may include molecules that can accept or release protons or ions. Such changes can be driven by pH changes or via added reagents that lead to pH changes or to ion formation or its reversal.

Ways of introducing such groups to protect the ß-amino ester environments are described in this document but involve use of selected combinations of donors and acceptors, pre-reactions of donors or acceptors with a modifying reactant (bearing those groups above), or post-shell formation reactions with a modifying reactant (bearing those types of groups above).

The term “modifying reactant or reagent” as used herein, refers to a reactive reagent which introduces such groups described into the polymer shell either directly via any post-reaction of the polymer shell, or via any pre-reaction of donors or acceptors going on to form the polymer shell.

The term “monomeric reactants” as used herein, are reactants which are small molecules, and which can react together to build up a polymeric structure. They may be difunctional or have higher functionality, or be multifunctional or polyfunctional, and may contain a portion of monofunctional molecules. In the context of this invention the monomeric reactants are donors or acceptors such as may take part in Michael Addition, or conjugate addition, reactions to build up molecular weight and so form a prepolymer or oligomer or a polymer.

The term “polymer” as used herein, refers to a compound comprising repeating structural units (monomers) connected by covalent chemical bonds. Polymers may be further derivatized, crosslinked, grafted, branched, or end-capped. Non-limiting examples of polymers include copolymers, terpolymers, tetrapolymers, quaternary polymers, and homologues. The term “copolymer” refers to a polymer consisting essentially of two or more different types of monomers polymerized to obtain said copolymer.

The term “pre-polymer”, “precursor”, and “pre-modified” as used herein, refers to any polymer or oligomer pre-made prior to the encapsulation process stage, and which can undergo some form of further chemical or physical transformation during or after the process of encapsulation such as a reaction (chain extension, branching, molecular rearrangement, crosslinking, ionic, complexation, or other linking or molecular association).

The release rate of the core material and the diffusion of the core material through the capsule wall can, in many cases, be controlled by varying the wall composition and/or the degree of crosslinking of the wall (shell) material. Also, the degree of crosslinking of the wall material directly impacts the strength and nature of the wall of the microcapsule. Furthermore, if a material is encapsulated, its useful life can be significantly extended. Also, if a material is toxic and/or difficult to handle, encapsulation of the material can reduce the threat of exposures and/or allow for easier handling.

Fragrances and oils and other lipophilic ingredients are widely used in personal and household care products such as detergents, fabric softeners, shampoos, and shower gels to enhance the product performance and attributes. Long lasting release of fragrances is a key performance parameter in many personal and household care products, yet many fragrances or oils are volatile, and their aroma effects are quickly lost on application. Encapsulation of fragrances inside a solid shell can protect fragrances and enable longer lasting release. Among the existing encapsulation systems, polymeric microcapsules made via interfacial polymerizations are widely used. These can be via oil-in-water (O/W) or water-in-oil (W/O) emulsions wherein typically, monomers react at the oil-water interface to form a polymeric shell. Most commercial fragrance microcapsules consist of poly(urea-formaldehyde), poly(melamine-formaldehyde), polyurethane, or polyurethane-urea shell materials or polyacrylates. Example References: U.S. Pat. 20080206291; WO2013092375; U.S. Pat. 20130337023; Chem. Eng. J. 2009, 149, 463; and WO 2017123965A1 are incorporated herein in its entirety.

These particular systems such as M-F systems, or U-F (urea-formaldehyde) or radically crosslinked acrylate or crosslinked urea or urethane systems, are chosen for their superior thermal and mechanical properties and are all rigid or highly crosslinked systems in order to retain volatile ingredients or ingredients that have a tendency to plasticize or dissolve away other shell walls or leach out through shell walls of other systems and also chosen for their stability in a wide range of end product formulations. They typically use low viscosity reactive monomers or reagents to enable interfacial or in-situ polymerization-encapsulation processes to proceed smoothly to form rigid, highly insoluble, and highly crosslinked polymer systems. They are designed for durability, including long lasting stability in various formulations over a range of pH's with salts or surfactants or solvents or other additives present which may compromise some other polymer shell walls, and have the feature of being friable meaning they are able to be crumbled or broken by application of pressure or friction such as in service or when required for release. This, for example is an attractive attribute in fragrance encapsulations for formulations or applications where a fragrance bloom (instant but long lasting or repeatable over time, release of cargo) can occur when shell walls are ‘crumbled’ or broken in service for example by rubbing or friction. Such durable polymeric shell materials are not claimed to be, nor would they be anticipated to be, biodegradable in aquatic environments, or indeed other environments such as soil or compost. Typically, such highly crosslinked or rigid polymer particles or capsules would be expected to be persistent or very slowly degrading in the environment and/or often use environmentally toxic or unfriendly materials such as formaldehyde or isocyanates in their production. Other routes such as coacervation do not make as robust a capsule and/or may require the use added undesirable solvents or use undesirable animal derived ingredients. There is a need for a robust microcapsule that can encapsulate, and retain until a triggered release, volatile or plasticizing cargoes and be biodegradable in the environments in which they end up in, which in many cases will be aquatic systems such as rivers, oceans or water treatment plants.

We have surprisingly discovered that volatile or plasticizing hydrophobic or lipophilic ingredients such as fragrances, oils and other lipophilic cargoes can be encapsulated within robust storage stable polymer shells which are biodegradable and yet which can display a similar or acceptable degree of cargo retention and bloom (release) to some non-biodegradable robust polymer shell options, and can in some cases also show longer lasting release of cargoes such as fragrances or oils or other lipophilic cargoes. Such polymer shells can be made using linear polymers or branched polymers, or using lightly crosslinked or, highly crosslinked polymer systems incorporating specific ß-amino-ester moieties in their backbones and/or in their branches and/or in their crosslinks. Furthermore, in addition to successful encapsulation and retention of potentially plasticizing or solvating lipophilic cargoes we have discovered that by selection of the polymer shell precursors and polymer architectures, such as linear or branched or crosslinked polymer shell systems, such polymer shell materials can meet important biodegradability criteria and in particular such criteria for biodegradability or non-persistence in ambient aquatic environments such as seawater, river/surface water, effluents, and other water treatment process streams (e.g. activated sludge). Furthermore, we have found that in some such combinations or systems the biodegradable capsules of our invention are also able to be stored ‘as is’ (as-made) or in formulated end products of various types and over a range of pH's and with common formulating additives such as surfactants or solvents or salts etc. also present. Thus, the invention encompasses a suite of microcapsule compositions, based on polymeric shell walls with specific ß-amino esters which can be designed to be biodegradable according to criteria herein described, and all of which have encapsulated lipophilic cargoes and are able to be tailored to meet the difficult combination of biodegradability, storage stability in formulated products, and triggered performance release or bloom of cargo, which span a range of performance levels suited to different formulated end products or applications and/or different encapsulated cargoes for those end product formulations. In addition, the invention also encompasses a suite of processes to produce said microcapsules.

The capsules of the invention are also able to be dried and stored dried and subsequently redispersed into formulations. They may also be formulated, directly as a slurry or after drying, into dry or ‘waterless’ formulated product forms such as tablets or soap bars or printed solid products other solid formats in various end applications, particularly, but not limited to, those used in personal and home care markets.

Biodegradation and non-persistence of materials which are in the environment are influenced by multiple factors. These include factors such as: (a) the environment in which the material finds itself either in use and/or after use, and the many factors therein such as, temperatures, humidity/water presence, pH, microbial populations, nutrients, etc., and (b) the timescale for monitoring or predicting biodegradation. Material composition, structure, morphology and physical size and form are also important factors, for a material of interest.

Evidence of biodegradation or non-persistence may be achieved via demonstrating a certain level of degradation within a time and/or degradation at a rate that is indicative that ultimately the material will degrade and be non-persistent after a certain time. There are many testing standards or methods which specify certain tests and associated timescales. In use there are some applications which may desire certain levels of biodegradation within certain timescales and environments to be met for evidence of biodegradation. Of course, for some applications a certain level or percentage (%) of biodegradation may be desirable or even stipulated as evidence of more rapid biodegradation or of a certain minimum level of biodegradation. Others may specify evidence of non-persistence. As to what evidence of biodegradation and/or of non-persistence is required or desired depends on the details of the end-application, the expectations of the customers (often the final product manufacturers) or of the consumers (typically, the end-users) and can vary according to end use and end environments or regulatory body directives or guidelines, of which there may be many variations. OECD, ISO, ASTM, EN or other standards for testing are commonly used to measure biodegradation or compostability and can be used as evidence of non-persistence or, indeed, conversely, of any likely persistence in an environment. However, they are not the only methods available and many publications report on other methods or criteria, and which are peer reviewed or rational to those skilled in the art. Furthermore, different end use sectors (products) and different regions of the world have different specifications or guidelines such that there is no single universal definition.

In the case of polymers, biodegradation typically begins with breakdown of the polymer chains or backbone into smaller components which continues until they become small enough to be intracellularly metabolized by micro-organisms such as bacteria, yeast, or fungi. Often the first steps of initial breakdown of polymers proceeds via hydrolysis or oxidation of the polymer backbone chains to generate smaller molecules suitable for intracellular consumption. Hydrolysis is a particularly common first step and may be facilitated, for example, by secreted extracellular enzymes (enzymatic hydrolysis; secreted by microorganisms in the end- or test-environment) and/or by the certain ambient conditions (pH, temperature, etc.).

Many polymers are resistant to biodegradation and persist in the environment for years or decades, for example many plastics, which are often used in applications for their long lasting durability. Similarly, many particles or microplastics are known to persist in the environments they end up in. This includes the many of the microcapsules of the prior art such as those based on M-F, U-F, crosslinked urea or urethane, and crosslinked polyacrylates. This has become a concern for the global environment such that nations and organizations, such as ECHA, may implement bans or restrictions on the use of microplastics that persist in the environment in certain products. In some respects, depending on the materials used, and their characteristics, microcapsules may be considered as a form of microplastics. As noted above microcapsules are very convenient for protection of cargoes (entrapped actives or ingredients) and/or controlled release of cargoes. Thus, biodegradable microcapsules are sought after.

Biodegradable capsules are known and particularly in the fields of biomedical and pharma applications. Common polymers include polyesters among others. Biodegradation in such applications is in physiological human (or animal) body environments and typically are at 37° C. and often with extremes of pH and/or a high presence of enzymes or nutrients that specifically facilitate breakdown of such polymers. Typically, the cargoes are solids, or water-soluble actives, and/or do not have volatile or reactive components. Also routes to manufacture such capsules involve undesirable solvents (such as dichloromethane) and/or processes such as microfluidics or freeze drying or evaporative processes, or extrusion methods, which are all impractical technically and/or commercially for encapsulating volatile fragrances and similar lipophilic cargoes or for applications in cosmetics or the personal care and household sector. Further, biodegradability in such biomedical/pharma environments (with their higher temperatures, enzyme presence, and more aggressive (for degradation) conditions etc.) is not indicative of, or comparable to, biodegradability in ambient aquatic waterways or seawater for example and, also, not reflective of the needs of the personal care or household sector, and other sectors (e.g. drilling/energy), where many of the products used will end in aquatic environments such as rivers, seas, surface water, water treatment plants/effluents—which are essentially ambient temperature (20° C. or lower) waters, or in soils or sediments.

It is reported that some of the microcapsules or other ingredients which are used in the personal care and household sector today may potentially be considered persistent in the environment and that they may fall under the umbrella definition of microplastics, and as such are undesirable. All such products as may be classed as microplastics are likely to be restricted in their use in personal care and household, and other products at some stage in the future. ECHA has initiated proposed processes for that. Other bodies may develop similar or alternative guidelines or protocols. Thus, there is a need to develop polymer capsules that are biodegradable in environments where common personal care and household products may eventually end up in. Demonstrating reasonable biodegradability of an ingredient will likely such avoid restrictions assuming other factors are also favorable. OECD and ISO test methods are typically specified for biodegradation testing in some cases. Other test standards are also used and are likely to be relevant and including future new standards as may be developed or specified. Thus, there is a need to develop polymer capsules that are biodegradable in environments where common personal care and household products, and many other products, may eventually end up in, and which can be manufactured in commercially sensible processes for that sector (so not using solvents requiring evaporation or high temperature encapsulation processes, for example).

In some OECD biodegradation tests for aquatic media, which are typically in relatively short timescales such as 28 days, under certain test conditions, achieving 60% biodegradation within 28 days in certain OECD tests can lead to a classification of being readily biodegradable. Such a material would be considered as rapidly biodegradable. In some OECD tests achieving 20% biodegradation can indicate a classification of a material being inherently biodegradable or primary inherently biodegradable. This indicates the potential for a material to be biodegradable, which would be over longer timescales than for readily biodegradable materials. Although 28 day tests are the standardized duration in some OECD tests, for the inherent classifications, extended testing periods of up to 60 days and longer if biodegradation has started within 28 days and has not yet reached plateau (see for example Annex 1 of OECD document OECD Guideline for Testing of Chemicals: part 1 Principles and strategies related to the testing of degradation of organic chemicals available at http://www.oecd.org/chemicalsafety/testing/34898616.pdf, in particular paragraphs 21 and 36).

Thus, for the purposes of this invention, evidence or data for biodegradability or evidence of non-persistence is tested in aquatic environments or media such as activated sludge, secondary effluent, river- or surface- or sea-water and the like according to OECD test standards but may be for longer than 28 days when biodegradation has started and not reach a plateau. Typically testing of biodegradation herein is according to methods of OECD or ISO test protocols, such methods and their variants as described for OECD 301, 302, 306, 310 or EN ISO 14852:2018 or EN ISO14851:2004 or EN ISO 19679:2016 or EN ISO 18830:2006 or EN ISO 17556:2012) or analogous or other standards. If in using such tests, about 20% biodegradation has been attained within 28 days or, is attained within a longer time period if biodegradation has started within 28 days and not reached a plateau, then that is provided as evidence for being biodegradable or non-persistent. Such evidence for biodegradation can be demonstrated within 28 days or 40 days, or 45 days or 60 days or 90 days or 3 months, or within 6 months, or within 12 months, or longer when tested according to standards, if no plateau is evident. Preferably for the purposes of this document 20% biodegradation will have been obtained within 60 days of such a standard OECD aquatic media and not shown a plateau in the biodegradation vs time plot. Thus, for testing purposes here, evidence or data for being biodegradable means evidence for inherently biodegradable or inherently primary biodegradable as per the OECD test methods and descriptions, including within longer timescale were allowed for in order to achieve 20% biodegradation with no plateau. It should be noted that not achieving such levels is not indicative of persistence—other tests can be applied to demonstrate non persistence or biodegradability in aquatic or other media. In addition, such OECD aquatic tests are typically at ambient conditions (20-25° C. or lower) and it will be recognized that biodegradability in other media (compost, soil, and sediments) will also be likely attainable if biodegradation in aquatic media is demonstrated. Also, ready biodegradability is also covered should it be demonstrated. Furthermore, OECD methods are not the only relevant test methods, although in this document they have been used for test data. Other criteria can be accepted and are used by others and in certain regions or applications. Other standard test methods or justifiable variations can be used, and other data may be accepted by industry regulators or by experts or if showing a sensible or logical rationale and/or where other evidence of non-persistence may be presented and accepted by those skilled in the art. For example, molecular weight reductions or weight loss or other measurements as evidence of biodegradation or non-persistence particularly for more slowly degrading materials may be used. Degradation half-life determinations are also be used. All are potentially relevant depending on the circumstances. This document reports biodegradation data using OECD test methods, though it is recognized such other tests or criteria may also be applied in to show biodegradation or non-persistence. For the many samples or material types which are insoluble in water, dispersions or films, other approaches are used to achieve reliable sample forms for biodegradation tests. It is recognized that today's test methods (OECD or other) for biodegradation of polymers in aquatic media are not necessarily representative, having not being designed or intended for testing such materials when originally conceived and especially for water-insoluble polymers and that refined or improved, or alternative test methods may in time be developed which are likely to be more relevant. (See for example: Kowalczyk, A. et al (2015) Refinement of biodegradation tests methodologies and the proposed utility of new microbial ecology techniques. Ecotoxicology and Environmental Safety, 111, 9-22. https://doi.org/10.1016/J.ECOENV.2014.09.021 and: Timothy J. Martin, et al (2017) Environmentally Relevant Inoculum Concentrations Improve the Reliability of Persistent Assessments in Biodegradation Screening Tests. Environ. Sci. Technol. 2017, 51, 3065-3073, DOI: 10.1021/acs.est.6b05717). It would be expected that if a material is showing evidence of biodegradation or potential non-persistence in the OECD or ISO tests reported in this document then it will also be biodegradable or non-persistent in future test specifications, likely more suited to polymers and the environments of today or the future. Also, where testing of polymers in seawater (marine), or surface/river water or activated sludge has shown some, even low levels of, ongoing biodegradability then such materials will likely show greater rates or degrees of biodegradation in more active media such as soil or compost, or other in media where enzymes or microorganisms are present in greater concentrations or diversity. It is reasonable to assume, and generally understood, that if biodegradability is shown in the usual aquatic media tests for a material, then the material would also be expected to be compostable according to the various standard tests for compostability. Furthermore, and similarly, it would also be reasonable to assume biodegradability in soil or similar media if shown to biodegradable in aquatic media. The reverse, however, is not able to be stated. Thus, if a material is confirmed as compostable, it is understood that it is not an indication that it will degrade in waterways or other ambient aquatic media. Polylactic acid is a well-known example of a polymer (polyester) that is compostable but will not biodegrade in aquatic media or soil. Thus, the testing in this invention is based on aquatic media on the basis that if a material is showing biodegradability in ambient aquatic media, it will also be compostable and degradable soil, according to typical standard test methods.

Thus in summary, it will be understood by those skilled in the art that for biodegradation testing the testing of biodegradability in aquatic media such as surface water or secondary effluent or activated sludge, as described by the aforementioned OECD tests, all carried out a temperatures around 20-25° C., is relatively mild and certainly less aggressive as a test for biodegradability compared to, for example, biodegradation testing in industrial composting facilities and via the test methods or standards developed for compostability testing such as EN13243 or ASTM D-6400 or ASTM D-6868, and others. In such compostability testing usual temperatures are much higher, for example around 58-60° C. It is understood that many polymers including polyesters such as polylactic acid which do show biodegradability in industrial composting tests are not able to show biodegradability in aquatic media tests (see for example: Bagheri, A. R., Laforsch, C., Greiner, A., Agarwal, S.: Global Challenges 2017, 1700048; DOI:10.1002/gch2.201700048). However, polyesters, or indeed other polymers, which do show evidence of biodegradation in such aquatic OECD tests would be confidently expected to be also compostable and able to pass tests for compostability.

A highly beneficial use, for example, of microcapsules is for the prolongation of fragrances or other ingredients which have been encapsulated inside a polymer shell. Typically, the technologies or materials used for encapsulation of fragrances or similar molecules (cargoes) have included melamine formaldehyde, polyurea/urethane technologies, or acrylate technologies. Most use crosslinked networks of these polymers for stability and durability in the formulations in which they are used (for example, laundry/washing products, household cleaning products, hair care products skin care products among others).

Thus, making microcapsules able to contain hydrophobic or lipophilic groups which may, also, optionally, be volatile and/or plasticizing, requires some alternative approaches to what is known in the prior art for making microcapsules suitable to encapsulate lipophilic or hydrophobic cargoes and yet which can also be storage stable and be biodegradable and especially biodegradable in aquatic environments such as seawater, rivers, surface water or in water treatment effluents, processes, or activated sludges.

It is an aim of this invention to meet these criteria and so enable production of microcapsules that have a shell material that is biodegradable or non-persistent, particularly in aquatic media/waterways, and yet which can retain a hydrophobic or lipophilic cargo or a volatile or a plasticizing or oil solubilized cargo such as a fragrance, an essential oil or any other oil, and is stable on storage in a product form until use. Fragrances are of prime interest since they are used in many end products and yet they typically have some volatile or low boiling components which can evaporate quickly if not contained in some way and/or components which are plasticizing to many polymers.

In terms of prior art, there are many patents and publications on microencapsulation of actives which are lipophilic. There are many examples of microencapsulations, of hydrophilic and lipophilic components for pharmaceutical or biomedical applications which describe biodegradable shells for controlled release. Biodegradation in such physiological environments is not representative of biodegradation requirements in aquatic waterways and the like. Physiological environments are typically warm at 37° C., have mixtures of specific degrading enzymes do not present in aquatic waterways for example, and/or have local pH extremes, and/or have salts and many other chemical entities also present. Overall, they are relatively aggressive media for degradation for controlled release. Furthermore, the shell wall materials, many of which are polyesters, and/or the processes typically used in drug or pharma active delivery are typically not suited to volatile or plasticizing cargoes. Many processes use extrusion (high temperatures), or solvents (requiring evaporation to very low residual limits) and when they do use undesirable components or reactants for shell walls (e.g. isocyanates for urethane shells) they will require significant cleaning or work-up to ensure removal of trace amounts of such components. Much pharma based encapsulations using polylactide or polyglycolide or poly(glycolide-co-lactide) (PLGA) polyesters as capsule shells use, for example, dichloromethane as an enabling solvent for encapsulations and it is necessarily subsequently removed by evaporation. All such aspects are not suited to volatile or plasticizing cargoes and/or are prohibitively expensive in their work up or other process stages for applications outside of pharma. For developments that are biodegradable shells of capsules, excepting those for pharmaceutical or biomedical applications, where, as just described, end environmental conditions (pH, temperature and/or presence of special enzymes etc.) are quite different from those in waterways and soil and where processes for manufacture are not well suited to those in personal or home markets, there are fewer in number and all of which have drawbacks inhibiting their widespread practical applicability. Our invention overcomes such drawbacks while also meeting the criteria described stated above.

In the existing prior art claiming poly-ß-amino esters for encapsulation, many show relatively rapid biodegradability in biomedical or physiological environments. Few have shown biodegradability in ambient aquatic environments or in related OECD tests, and those that do show such biodegradation are typically showing quite rapid biodegradation or ready biodegradation which is also indicative of instability on storage in aqueous or acid media. This is a concern, and indeed a major obstacle for wide commercial deployment, if the microcapsules were to be used in a liquid laundry or cosmetic or personal care product formulation. Common processes to date to make poly ß-amino-ester capsules make the poly-ß amino-ester in-situ in the presence of the cargo via interfacial polymerizations where one reactive or catalytic ingredient is in the water phase and another in the oil phase. None actually show a combination of such biodegradation properties with a successful encapsulation of a fragrance or similar volatile lipophilic cargo with the attributes of imparting a noticeable bloom or release of cargo when triggered (e.g. when rubbed or application of pressure). Furthermore, few if any show any ability to be stable on storage as made with fragrance or oils or other plasticizing cargoes inside or show stability on storage in aqueous media or aqueous end product formulations, as are used in home or personal care applications which may have pH extremes or surfactants or salts or solvents or other additives that may plasticize or attack the shell wall.

In one embodiment, the present application provides a microcapsule comprising: (i) a polymeric microcapsule shell; and (ii) a lipophilic core; wherein, the polymeric microcapsule shell comprises a poly-ß-amino-ester polymer, co-polymer of poly-ß-amino-ester, terpolymer of poly-ß-amino-ester, a crosslinked polymer of poly-ß-amino-ester, or mixtures thereof, wherein the microcapsule is storage stable, and its polymeric shell is biodegradable. The poly-ß-amino-ester ester based capsules of our invention show successful microencapsulation and subsequent triggered release of fragrance or other lipophilic cargoes with associated evidence for biodegradability or potential non-persistence, in aquatic media according to OECD test methods and are made via convenient in-situ polymerization processes at low to moderate temperatures suited to volatile ingredient encapsulations, and not requiring subsequent volatile solvent removal or the use of undesirable isocyanate or formaldehyde or acid chlorides or the use of high temperatures at the encapsulation stage, and do not necessarily use excess reactants that remain unreacted.

Poly-ß-amino ester homopolymer complexes, particles and capsules have been described. The polymers are typically made by Michael Addition, or conjugate addition, reactions of a difunctional or multifunctional amine donor (bearing primary or secondary amines which is at least difunctional on available NH groups, and a difunctional or multifunctional acceptor (e.g. an activated (electron deficient) conjugated double bond as in an acrylate or related molecules, well known in the field). Solvent based have been applied to make polymers or capsules methods are applied, e.g. with water as a solvent, typically making hydrogel based encapsulations from such precursors. Other solvent mediated processes, or classical interfacial polymerizations (oil-in-water polymerizations wherein the donor is one phase and the acceptor and/or a catalyst is either in the other (water) phase or is added as a polar phase to a preformed oil in water emulsion made from the one of the (oil soluble) reactant streams and a water phase) have also been applied, though less frequently, to make capsules from these polymers and the precursor Michael Addition reagents. These known methods for poly-ß-amino-esters have disadvantages when trying to encapsulate polar or volatile or plasticizing cargoes in a robust, highly cross-linked, or rigid shell.

When a water-soluble donor is reacted with a water-soluble acceptor in water typically hydrogel matrix capsules result. These are suited to controlled release over time of drugs, for example. Hydrogel matrix capsules are generally are not as retentive or robust as core shell capsules and, as made, are not well suited to encapsulation of ingredients or actives (such as a fragrance for example) where a triggered more instant release of cargo is desired. Also, they would not be suitable for storage in aqueous based formulated products requiring long term retention before release of their cargo. When solvent mediated processes are used for encapsulations with such reagents the solvents will typically need to be removed (typically evaporation and/or other complicated double emulsion or other processes will be required), and, as described above, these are not well suited to volatile cargoes or to commercially viable processes for personal or home care applications. In addition, much of the prior art describes poly-ß-amino esters for biomedical/pharma environments and as useful in these media for their very rapid degradation—good for biodegradability profiles but not well suited to storage (until required use) in aqueous formulations as used in personal or home care applications.

Classical interfacial oil-in-water polymerizations (and encapsulations) have been described for poly-ß-amino-esters and related polymers. Typically, an oil soluble acceptor (e.g. a di- or multi-functional acrylate acceptor) is emulsified with an aqueous phase and a donor (e.g., a di- or multi-functional amine) is added to the pre-formed emulsion as a polar phase, typically in significant excess, to react at the interface to form a polymer, or, in some variants, a water-soluble donor (amine) is incorporated into the water phase when forming the oil-water emulsion. Interfacial polymerizations with such systems proceed typically with the cargo and one monomer (acceptor here) in an oil phase and emulsified with a water-surfactant mixture to make a pre-emulsion. The second monomer (e.g. amine donor) is not dissolved in the oil phase for this process. The interfacial polymerization reaction proceeds forming a shell around the cargo at the interface. Such interfacial polymerizations have disadvantages for encapsulations of some cargoes, including those that are more polar or plasticising or volatile molecules, and including many natural or essential oils or fragrances or other hydrophobic or lipophilic cargoes, for several reasons; first, an excess of donor (amine) tends to be required, leading to, for such interfacial polymerizations, the presence of residual (unreacted) monomers or residual unreacted functional groups, possibly requiring more rigorous washing or clean-up processes at the end of reaction. This can be inconvenient, wasteful, and expensive for commercial processes since very low residual amounts of such reactants for personal or home care products will typically be required.

Secondly, donors such as water-soluble amines are relatively hydrophilic, and therefore the resulting polymers tend to be more swellable or softer in water-based systems or potentially more hydrolytically labile or sensitive, and so potentially leakier when stored in aqueous media (as made (slurries) for example) or when formulated in aqueous media, especially at pH's away from neutral, as is common in many applications in laundry or home care or personal care for example. This can negatively impact cargo retention (e.g. fragrance or oil or other hydrophobic cargo) and storage stability across a wide range of pH's retention especially where release of cargo is not sought to be gradual over time or is required to be delayed until a trigger for release is applied. Thus, the use of polar, water-soluble/hydrophilic polyamines and similar hydrophilic donors can make such capsules too hydrolytically unstable, leading to premature degradation in aqueous media (e.g. as made and/or when formulated)—and so again leading to capsules which are less storage stable as made in aqueous media, and/or at pH's away from neutral such as acidic pH3. Whilst, as noted above, this may be advantageous for biodegradation performance in some circumstances, or for controlled release over time, this is a major disadvantage when longer term storage of capsules is required or desired, as an aqueous dispersion or slurry (the form they are typically made in), or in formulated products (e.g. liquid detergents, fabric conditioners), which are aqueous and may have pH extremes—and where a subsequent triggered release (e.g. by rubbing/friction) is desired. Fourthly, if very high crosslink densities such as those systems with both acceptor and donor having very high multifunctionality (e.g. tri-functional or higher for each reactant) were to be required for capsule shells, as is the case for some applications described in our invention herein where particularly robust capsules are needed, the classical interfacial polymerization route can be very limiting since as the two high functionality donor and acceptor molecules co-react and polymerize at the interface they will quickly form a highly crosslinked shell structure relatively early on in the reaction zone and this prevents migration or diffusion of further amine donor into the polymerizing zone—so limiting donor and, consequently acceptor (e.g. acrylate) conversion. This may also limit the attainment of higher crosslinked structures for the shell, which are required for more robust (retentive until broken and/or stable on storage) capsules and leaves relative high levels of unreacted functional groups or monomeric reactants. This latter occurrence is one reason why it is usually required to use excess donor (e.g., amine or thiol) in such a classical interfacial polymerization, which as mentioned above has such significant drawbacks.

For the prior art relating to microcapsules from poly-ß-amino esters all such prior art where classical oil-in-water polymerizations are described for microcapsule formation follow classical interfacial polymerization processes which have such limitations in achieving the higher crosslink density requirements for the most demanding of end product applications and need to use excess addition levels of one of the reactive reagents. Furthermore, in such prior art, there is a need for one of the monomer reactants to be, initially at least, outside of the oil phase. Also, none of the prior art on such ß-amino-ester microcapsules show a combination of proven storage stabilities, with relevant biodegradation properties and with a successful encapsulation of a cargo with the attributes of imparting a noticeable bloom or release of cargo when triggered (e.g. when rubbed or application of pressure) after storage or after incorporation into a formulation. No prior art describes an in-situ oil-in-water polymerization process (where all reactants are in the oil phase at the outset) for their production and its associated advantages for achieving the desirable balance of selected reactants and desired crosslink densities for the most demanding of applications. Furthermore no prior art shows the use of poly-ß-amino ester microcapsules made by any emulsion polymerization process, for retaining volatile or aggressive lipophilic cargoes such as fragrances or perfumes or essential oils, which achieve a balance of long term storage stability and biodegradation in aquatic media, and in which the poly-ß-amino ester or microcapsule shell composition comprising a poly-ß-amino ester is specifically composed of hydrophobic, sterically hindered, charged or pH responsive groups.

The poly ß amino-ester based microcapsule shells of our invention show successful encapsulation and subsequent triggered release of fragrance, and have storage stability in aqueous formulations, and can demonstrate associated evidence for biodegradability or non-persistence over time in aquatic media according to OECD test methods. Furthermore, they are made via a convenient emulsion processes including, for example, an in-situ process, not requiring substantial excess amounts of reactants, which can be conducted at low to moderate temperatures suited to volatile ingredient encapsulations in an oil-in-water process, and not requiring subsequent volatile solvent removal and not using undesirable isocyanate or other such reagents nor requiring high temperatures at the encapsulation stage. Furthermore, they show a combination of fragrance encapsulation, biodegradability and storage stability in aqueous media or various formulated products or pH ranges.

Present invention relates to biodegradable microcapsules, particularly, microcapsules that: (a) can encapsulate and retain cargoes, which can subsequently be released by a trigger and/or released gradually, and particularly where such cargoes are, or contain, lipophilic or hydrophobic core materials such as fragrances, butters or essential oil or other oils or oil solubilized cargoes; and, (b) whose shell material(s) show evidence of biodegradation or non-persistence in the environment and in particular in environments that are aquatic based (waterways, rivers, surface waters, seawater, sludge, treated waters, etc.) and/or soil or compost based and (c) which are storage stable as made or in one or more end-product formulations.

Present application further describes a route to make micron sized (and above) capsules (microcapsules) and can be used for encapsulating sensitive or plasticizing or volatile lipophilic or other hydrophobic ingredients or actives or such as oils, or fragrances or butters or oil solubilized ingredients. Said biodegradable microcapsule polymeric shell compositions can effectively be used in various applications including, but not limited to personal care products, home care products, etc.

Our approaches have surprisingly found that polymeric shell capsules can be made to encapsulate fragrances, oils etc. and other cargoes which exhibit lipophilic tendencies, compatibilities, or behaviors and which are stable on storage in aqueous media such as ‘as-made’, or in aqueous formulations of various pH's and optionally containing surfactants or other additives, and yet which are able to biodegrade in common, ambient, water based environments after use. Insoluble materials can be encapsulated by dissolution or partial dissolution, or via dispersion or emulsification, in a lipophilic carrier or diluent additive.

Non-limiting examples of cargoes that can be encapsulated through any of the embodiments in addition to fragrances, perfumes, essential or natural oils and the like, including oil (ester or hydrocarbon) solubilized ingredients, liquids or low melting solids include lipophilic esters, chlorinated solvents, hydrocarbons, insect repellants, and pigments, colorants, dyes, vitamins, antioxidants, lipophilic natural extracts, or other actives which are oily or oil (ester or hydrocarbon) soluble.

In another embodiment, the present application provides various methods for preparing said microcapsules and for preparing microcapsules from any combination of multifunctional amine donor and multifunctional acceptors such as acrylates, methacrylates, acrylamides, methacrylamides, itaconates, or maleates or fumarates, and others.

In another embodiment, the present application provides a prepolymer, which is biodegradable in the chosen medium (such as seawater, river water, activated sludge, etc. or soil or compost) is synthesized, or pre-synthesized with reactive groups either in-chain or at chain end(s). Here the term prepolymer is used to describe any polymer or oligomer pre-made prior to the encapsulation process stage during which it is transformed to form a microcapsule shell, and which is biodegradable or hydrolysable and which is also initially compatible with the heated cargo (or cargo diluent mixture) as described below. A prepolymer containing ß-amino-ester bonds, optionally with amide and/or ether and/or ester and/or carbonate and/or urethane bonds, is preferred, though ensuring its structure and composition is necessarily biodegradable according to criteria herein described. The pre-polymer may also be a polymer with similar links (amide, ether, ester, ß-amino-ester, carbonate, etc.) but designed to be biodegradable, with suitable reactive functionality, in chain or at chain ends, to then react as either donor or acceptor in a reaction (with acceptor(s) or donor(s)) to form poly ß-amino-ester microcapsules.

The pre-polymer is melted or dissolved (with warming if needed) into the cargo (optionally with added diluent or carrier) and so is also necessarily designed to be compatible with the cargo or a mixture of the cargo and a diluent, when heated. Optionally co-reactive reagents (that may react with the reactive groups, in-chain or at chain ends and/or aid solubilization) or free radical initiators and/or other catalysts or accelerators may also be incorporated and/or additives for example that enable formation of complexes, salts, or other forms of interactions with the pre-polymer to transform it during the capsule shell formation process. An inert (that is not necessarily co-reacting) biodegradable polymer additive may also be incorporated as an option, so making a polymer shell wall with a blended mixture of polymers.

The prepolymer may contain excess functional groups such as amine or acrylate, such as for example, in the case of a poly-ß-amino-ester prepolymer, arising from selected stoichiometries of the monomeric reactants that may have been used in the preparation of the poly-ß-amino-ester prepolymer, and/or may contain other reactive groups, such as unsaturated groups, at chain ends or distributed along the chain, which can be used for the transformation of the prepolymer during shell wall formation. Non-limiting examples of reactive unsaturation functionality include acrylate, methacrylate, acrylamide, methacrylamide, itaconate, citraconate, maleate, fumarate, crotonate, and combinations thereof.

The prepolymer cargo mixture (oil phase, with optional diluent) is mixed with an aqueous phase which can be solely water or water with added stabilizers or other additives. Optionally co-reactive reagents (that may react with the reactive groups, in-chain or at chain ends) or free radical initiators or crosslinking initiators are also incorporated and/or additives that may enable formation of complexes, salts, or other forms of interactions with the prepolymer. The mixture is homogenized or stirred vigorously to form an emulsion while warm or heated and reacted.

The capsules are formed during the reaction with stirring or homogenization. An insoluble polymer (insoluble in the cargo and insoluble in water) shell wall is formed for example via solidification or precipitation of the polymer or prepolymer on cooling and/or via crosslinking or chain extension or branching reactions between prepolymer molecules and/or between prepolymers and added co-reactive reagents, and/or via molecular rearrangements, and/or via complexation or formation of ionic salt bonds or interactions.

Mixtures of stabilizers may be used (and incorporated at various points) and mixtures of stabilizers with added polymers to complement them and/or aid control viscosity or stability.

Non-limiting examples of stabilizers used alone or as part of a mixture, include polyvinyl alcohols, polyvinylpyrrolidones, hydroxyethyl celluloses, hydroxypropyl celluloses and other cellulosic derivatives, guar, guar derivatives including cationic guars, gums including xanthan gum and the like, starches and starch derivatives, and/or any known emulsifier or dispersing aid and including particles such as silicas. Particle stabilized (Pickering emulsion) approaches are also able to be used.

Non-limiting examples of defoamers may also be used which may include liquid hydrocarbons, oils, hydrophobic silicas, fatty acids, alkoxylated compounds, polyethers, polyalklylene glycols, and nonionic emulsifiers.

This process outline and its incorporated variations can be applied to produce a microcapsule which has a shell wall which is biodegradable in the chosen medium and yet can encapsulate and retain a lipophilic cargo.

The microcapsule according to the above descriptions is formed wherein a polymer shell is formed around the cargo by a process of solubilization of a biodegradable pre-polymer or pre-oligomer, containing ß-amino-ester bonds, optionally with amide and/or ether and/or carbonate and/or urethane bonds, in a lipophilic cargo, optionally with added diluent or other reagents and/or heating, then emulsifying with water, and effecting a transformation of the pre-polymer such that it becomes insoluble in the lipophilic cargo, such a transformation being effected either via reactions, molecular rearrangements or interactions, and/or phase or solubility transitions of the pre-polymer within or from its mixture with the fragrance and diluent, if present and yet wherein such transformed polymer (capsule shell wall) encapsulates the cargo and still remains biodegradable or non-persistent in the environment.

Non-limiting examples of a diluent or solvent is selected from the group consisting of hydrocarbon oil, alkanes, an ester oils, a fatty acid esters, an aliphatic esters, and alkylene carbonates.

A microcapsule with a biodegradable shell wall is produced according to any of the above methods, wherein such a polymer shell is formed around the cargo. Accordingly, the lipophilic core is selected from the group comprising agrochemicals, aliphatic esters, anti-microbial agents, anti-fungal, anti-fouling agents, antioxidants, anti-viral agents, biocides, catalysts, cosmetic actives, dyes, colorants, detergents, edible oils, emollient oils, essential oils, fats, fatty acids, fatty acid esters, food additives, flavors, fragrances, hair care actives, halogenated compounds, hydrocarbons, insecticides, insect repellants, lipids, lipophilic scale inhibitors, mineral oil, oral care actives, organic solvents, organic esters, chlorinated solvents, pesticides, perfumes, preservatives, skin care actives, UV absorbers, vegetable oils and combinations thereof. Accordingly, the core is a fragrance, a perfume, or an essential oil.

In some cases, the products may be particles with entrained or absorbed or adsorbed cargo rather than fully formed capsules or may be capsules which function in both aspects. Entrained or absorbed cargoes are still retained though typically for shorter times compared to fully encapsulated cargoes in shell walls. A combination of entrained, absorbed, or adsorbed cargo together with encapsulated cargo is also able to make in some cases. Also, the capsules or particles may form films on drying or casting or other processing which also contain and retain the cargo for certain times, all still being biodegradable. In one embodiment, microcapsules that are formed in a slurry (typical initial reaction product mixture) with encapsulated cargo which can dry as capsules and then, if desired, be re-dispersed in water or aqueous media or formulations and retained as capsules which are biodegradable.

In some embodiments, present application provides a prepolymer or pre-modified (reacted) donor or acceptor, so modified or chosen to introduce specific attributes or features, which is subsequently used in an oil-in-water microencapsulation process to make a polymeric shell around a cargo, which has specific attributes or features. In other embodiments, the polymer shells are built up during oil-in water reactions of selected monomeric reactants, or mixtures thereof, chosen to introduce specific attributes or features. Other methods to make the polymeric shells with the specific attributes or features targeted are also able to be used. In some other embodiments the polymer shell, made by any method, is modified post (after the) encapsulation reaction to introduce specific attributes or features.

In all the embodiments described above, the polymeric shells based on poly-ß-amino-esters further comprise hydrophobic or sterically hindered or charged or pH responsive functional groups. Hydrophobic groups refer to water repelling and inhibit the access of water molecules to the ß-amino ester bond environment. They tend to be non-polar substituents, such as hydrocarbon chains. The hydrophobic groups may be within another molecule, for example a saccharide or polysaccharide, a protein, a polyol, a sugar alcohol, or other molecule containing hydrophobic groups, and which becomes linked to the poly-ß-amino ester (or copolymer). Linking reactions with the poly-ß-amino ester (or copolymer) may be via free radical routes, or via Addition Reactions, or via other routes which reactively link the poly-ß-amino ester (or copolymer) with functional groups on the saccharide, protein, polyol, sugar alcohol or other molecule containing the hydrophobic modifying moiety. Alternatively, or in addition, the introduction of the modifying group can be via an overcoating step applied to the polymeric (poly-ß-amino ester or copolymer) capsule shell. The same routes can be applied to introducing sterically crowded or charged or pH responsive groups which may be on saccharides or proteins or other molecules.

Sterically hindered means relatively crowded, bulky or sterically shielding. Sterically hindering groups are groups which ‘crowd out’ or shield the ß-amino ester bond environment so that the ß-amino-ester bonds are partially shielded by neighboring groups and so water or larger molecules cannot easily approach and so react with the ß-amino ester bonds. They may also have the effect of reducing the ability for bond rotations in that environment. The net effect is similar to hydrophobic groups in inhibiting the access of water molecules to the ß-amino ester bond environment. They tend to be cyclic, bulky, or branched (especially tertiary alkyl) groups. As such many sterically hindering groups used herein used are also hydrophobic groups and vice versa. A relative measure of that might be a reduced water coordination number around the ß-amino ester bond environment. A lowered water coordination compared to a model or typical linear poly ß-amino ester can be one factor, other things being equal, indicative of a lower likelihood of a hydrolysis reaction with water, and so more storage stable in aqueous formulations for example. In other embodiments the sterically crowding group can be a saccharide including a monosaccharide, disaccharide, oligosaccharide, or a polysaccharide which due to its ring structure can be sterically crowding. Such saccharides may additionally contain hydrophobic or charged groups.

Non-limiting examples of hydrophobic or sterically hindered groups are selected from the group consisting of methyl, ethyl, propyl, butyl, C₅-C₂₀ alkyl, C₅-C₂₀ branched alkyl, tertiary methyl, tertiary ethyl, tertiary propyl, tertiary butyl, cyclohexyl, alkyl-cyclohexyl, isobornyl, norbornyl, menthyl, cholesteryl, cycloaliphatic, phenyl, phenoxyethyl, benzyl, and aryl moieties. Saccharides such as glucose, galactose, fructose, sucrose, maltose, lactose, xylose, trehalose, guar gum, other saccharide based gums, pectin, starches, hyaluronic acid, and variants or derivatives of such saccharides, including sugar alcohols, with charged or hydrophobic groups, are all sterically crowding and/or (depending on their structural features) charged and/or hydrophobic modifying groups when attached to the amino-ester molecules.

Charged moieties bear a positive or a negative charge, as is well known for certain classes of molecules in organic chemistry. “pH Responsive”, or “pH stabilizing”, or “pH sensitive” (pH responsive is used to cover all such terms or implied variations) are molecules or groups that can change its structure or shape in way to be stable in a pH extreme way from neutral. Typically, this is by accepting or releasing a charge (positive or negative) or may be via a change in hydrogen bonding. pH Responsive groups can be acidic groups (such as —COOH, —SO₃H, or others) or basic groups (—NH₂, —NR₂, or others). The mechanism of response is the same for both in that either a gain or loss of a negative charge or a positive charge is involved. This might typically involve either deprotonation or protonation, or salt or ion formation, depending on the groups and the pH extreme experienced. Moieties stable in or respond to pH extremes or pH changes such that the poly-ß-amino ester polymeric shell achieves a desired balance of biodegradability—storage stability in aqueous media or formulated products—and a triggered cargo release.

Non-limiting examples of charged or pH responsive functional group is selected from the group comprising carboxylic acid, carboxylate, sulfonic acid, sulfonate, phosphonic acid, phosphate, boronic acid, borate, quaternary ammonium, ammonium, tertiary amine, secondary amine, primary amine, amine containing heterocyclic bases, amino-acids, salts, and conjugates derivatives thereof, or may be modified saccharides or modified saccharide derivatives.

In another embodiment, the present application provides an in-situ oil-in-water polymerization process, with all reactants in the one (oil phase) is used to make poly-ß-amino ester.

Schemes 1, 2 and 3 below illustrate some of the aspects or embodiments of the invention.

### Scheme I

Example scheme for a pre-reaction of an amine donor to make a modified amine donor which is then used in the encapsulation reaction stage via a further addition reaction with multifunctional acrylate or other acceptor, for microcapsule formation which has a wall made from a novel poly ß-amino ester of the invention bearing one or more specific attributes or features (introduced via ‘X’) where X is hydrophobic, sterically hindered, charged or pH responsive. Other pre-modification reactions are also feasible to introduce the same attributes or features. Scheme-I is represented below:

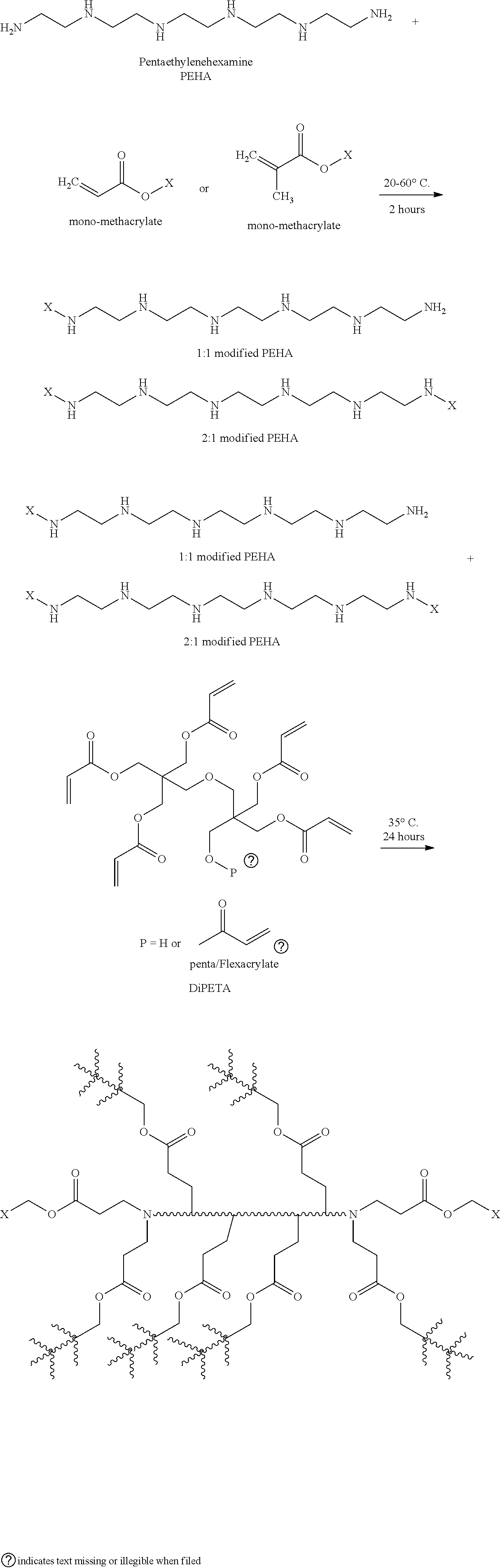

### Scheme II

Example scheme for a post-reaction modification of a microcapsule shell to make a modified capsule shell in this case via an addition reaction with mono-functional acrylate or methacrylate or other acceptor, to produce a modified microcapsule which has a wall made from a novel poly ß-amino ester of the invention bearing one or more specific attributes or features (introduced via ‘X’) where X is hydrophobic, sterically hindered, charged or pH responsive. Other post-modification reactions are also feasible to introduce the same attributes or features. Scheme-II is represented below:

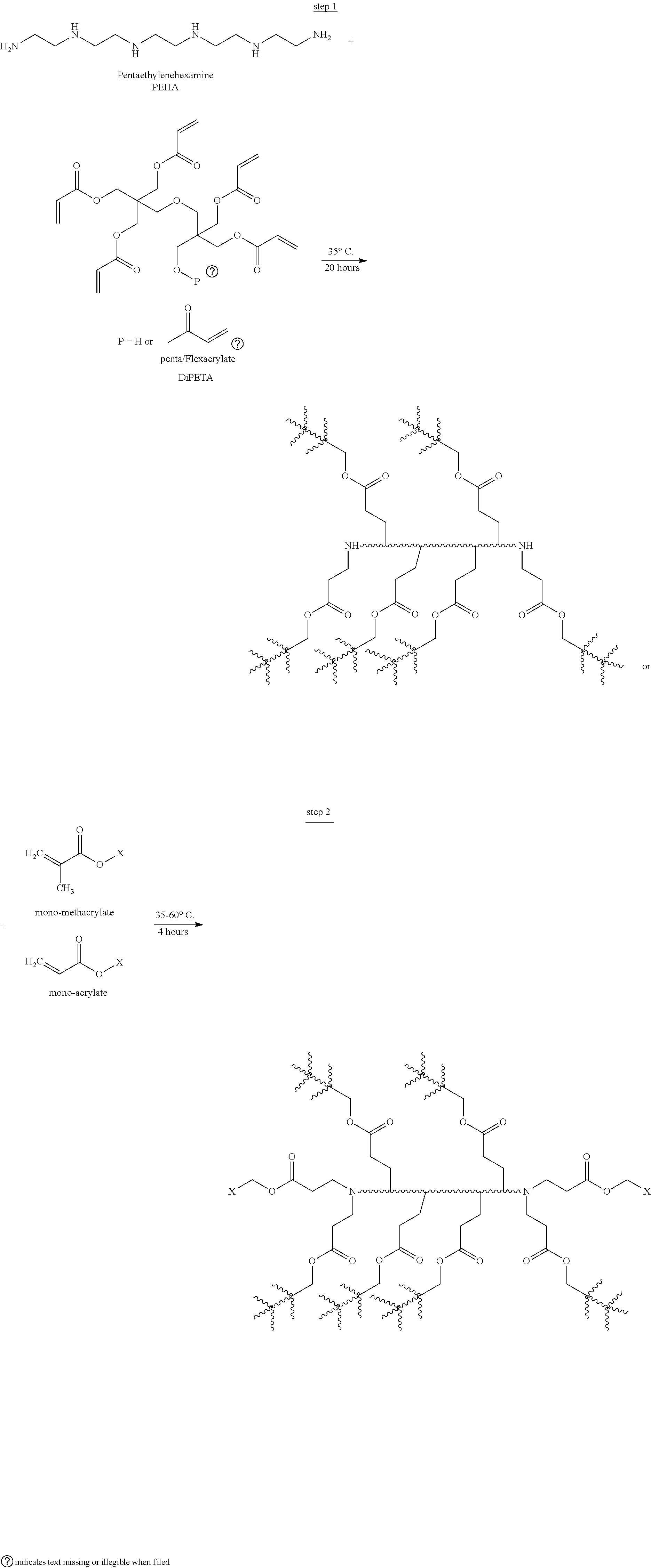

### Scheme III

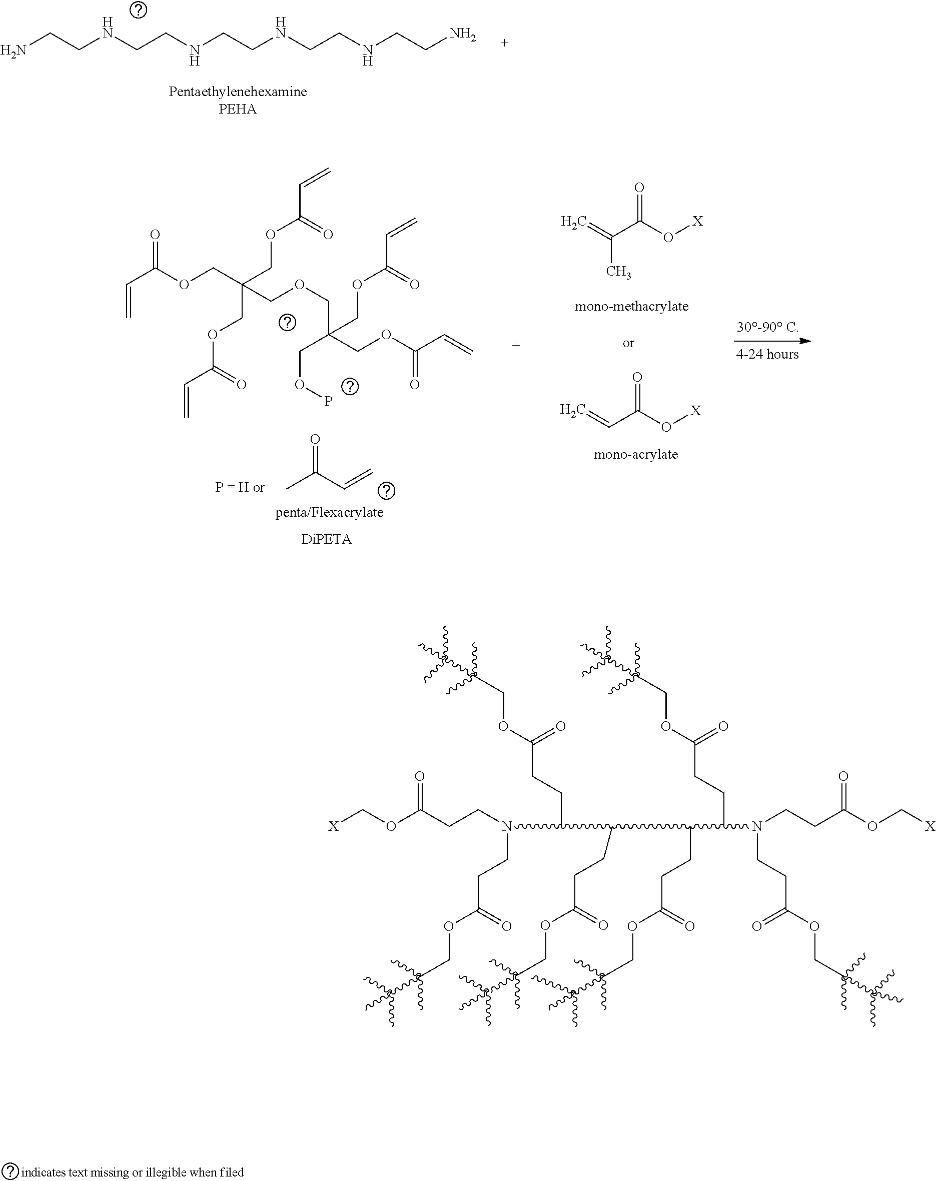

An example Scheme (III) is shown above for a reaction of selected mixed monomeric reactants, here of a multifunctional amine donor with a mixture of acceptors where at least one component is a hydrophobic, sterically hindered, pH responsive, charged acceptor, and at least one component is a multifunctional acceptor. Here below, as an example, a mono-functional acceptor is included with a multi-functional acceptor. Such mixtures are used in the encapsulation reaction wherein addition reactions proceed between donors and acceptors to produce a microcapsule shell wall based on a novel poly ß-amino ester of the invention bearing one or more specific attributes or features (introduced via ‘X’) where X is hydrophobic, sterically hindered, charged or pH responsive. Other monomeric reactant combinations are also feasible, for example using amine donors incorporating such features or such features also being present in multifunctional acceptor component to introduce the same attributes or features.

Although in Schemes I and II the modifying reactions (pre- or post-) are also (in addition to the capsule shell polymer forming stage) Michael Additions using donors and acceptors they can, as described further below, be other reactions that introduce the same functional groups or features into the polymeric shell material.

In some cases, such features (X) may be inherent in a multifunctional monomeric reactant. Thus, the same or similar effects can be achieved with other selected mixed monomeric reactants (chosen to introduce the specific attributes or features) present from the start, though for the most demanding applications a pre- or post-modified approach has been found to have some advantages in performance or stability.

In another embodiment, the present application provides various routes to prepare capsules (microcapsules) of the invention, which can contain, retain, or entrain a hydrophobic or lipophilic cargo, such as a fragrance or oil, and which can also be biodegradable in aquatic or other environments. As described above, the prior art also describes either use of small molecule monomers or precursors reacting in-situ, in an emulsion or dispersion process, to form a polymer or crosslinked polymer network in-situ from small molecules (monomers) such as acrylate monomers, or melamine-formaldehyde (M-F), or isocyanates with diols or diamines (for polyurethanes or polyureas) for encapsulating oils or fragrances with good retention. Here prepolymers are not necessarily made or required. We have discovered that biodegradable polymer shells for microcapsules encapsulating lipophilic cargoes such as fragrances oils and the like and which polymer shell walls comprise ß-amino-ester bonds, optionally with amide and/or ether and/or thioether and/or carbonate and/or urethane bonds present, can also be made through small molecule precursor (monomers) routes and not necessarily require a prepolymer to be made, though prepolymers may also be present in such approaches.

In such methods introducing such functional groups, we have surprisingly found that certain poly ß-amino ester shells, useful as microcapsule shells, that can be designed to be storage stable and be biodegradable according to criteria described herein, and which can contain, retain, or entrain fragrances or other lipophilic cargoes or oil solubilized cargoes, can be made. For example they can be made by an in-situ polymerization-encapsulation emulsion (oil in water) polycondensation process starting from monomeric precursors such as difunctional or multifunctional amines and difunctional or multifunctional acceptors such as acrylates or methacrylates without the need for reactions at higher temperatures, in the presence of fragrances and other lipophilic cargoes (oil phase), and with all monomeric reactants distributed or placed into in the oil phase from the outset.

In another embodiment, we have also discovered that, surprisingly, that certain highly branched or crosslinked polymeric shells that comprise ß-amino-ester moieties and where the shell composition or material comprises components with such functional groups, and contains, retains, or entrains fragrances or other lipophilic cargoes or oil solubilized cargoes, can be designed to be storage stable and biodegradable according to criteria described herein. For example, they can also be made by an in-situ polymerization-encapsulation emulsion (oil in water) process, with all monomeric reactants substantially in the oil phase from the outset, without the need for water soluble precursors and/or without the need for large excesses of reactive monomers and/or without the need for long reaction times at higher temperatures.

In all embodiments, the poly-ß-amino-ester forming, or present in, the microcapsule shell is derived from a Michael or conjugate Addition reaction of at least one amine donor and at least one acceptor, wherein at least one donor component and at least one acceptor component each have a reactive functionality of at least two. Accordingly, the poly-ß-amino-ester, derived from a Michael or conjugate Addition reaction of at least one amine donor and at least one acceptor, wherein at least one donor component and at least one acceptor component each have a reactive functionality of at least two or at least three.

In a non-limiting embodiment, polymeric microcapsule shell is derived from a donor-acceptor combination selected from the group containing: (i) a difunctional, trifunctional, tetrafunctional, pentafunctional or hexafunctional amine; and (ii) a difunctional, trifunctional, tetrafunctional, pentafunctional or hexafunctional acrylate or methacrylate.

In all embodiments, the donor is an amine or has an amine as a major component. The donor can be a mixture of at least one difunctional amine and/or multifunctional amine. The amine donor is a difunctional primary amine, a multifunctional primary amine, a difunctional secondary amine, a multifunctional secondary amine, or combinations thereof. Accordingly, the amine comprises a C₂-C₂₀ aliphatic chain, a C₄-C₇ cyclic ring or a C₄-C₇ heterocyclic ring.

In another embodiment, the amine donor selected from the group consisting of any primary alkylamine or primary cyclo-alkylamine, 4,4′trimethylenepiperidine (TMPP), isophorone diamine (IPD), bis-(aminomethyl) cyclohexane, cyclohexane diamine, piperazine, aminoethyl piperazine, bis-amino-norbornane, ethylene diamine, diethylene triamine, diethylene diamine, triethylene tetramine, tetraethylene pentamine, pentaethylene hexamine (PEHA), tris(2-aminoethyl) amine, bis(3-aminopropyl) amine, spermine, hexamethylene diamine (HMDA), diamino-propane, diamino-butane, diamino-pentane, diamino-octane, diamino-decane, diamino dodecane, amino-ethanol amino-propanol, amino-butanol, amino pentanol, any polyfunctional amine, and polyethyleneimine or any derivatives of such di- or multi-functional amines with available amine groups for reaction with acceptors.

In another embodiment, the one acceptor is selected from the group consisting of: (a) an itaconate containing polyester, (b) an acrylate, diacrylate, or multifunctional acrylate of a polyester; (c) an acrylate, diacrylate, or multifunctional acrylate of an epoxide; (d) an acrylate, diacrylate, or multifunctional acrylate of a urethane; (e) an acrylate, diacrylate, or multifunctional acrylate of a polyether or polyol; (f) an acrylate, diacrylate, or multifunctional acrylate of an amine; (g) methacrylate analogue of (a) to (f) components, or combinations thereof.

Non-limiting examples of acrylates are selected from the group consisting of butanediol diacrylate, trimethylol propane triacrylate, pentaerythritol triacrylate, pentaerythritol tetra-acrylate, dipentaerythritol penta-acrylate, and dipentaerythritol hexa-acrylate, and methacrylate analogues thereof.

In another embodiment, the present application provides donor-acceptor combination comprising difunctional amine, trifunctional amine, tetrafunctional amine, pentafunctional amine or hexafunctional amine. These may be primary or secondary amines. The acceptor or donor component is a monofunctional acceptor or a monofunctional donor.

Another embodiment discloses that the crosslinked polymer comprises combinations of amines to make specific ß-amino ester structures with enhanced performance in fragrance release in formulated products and/or enhanced storage stability in formulated products or aqueous systems. Accordingly, the polymeric shell comprises a crosslinked poly ß-amino-ester polymer.

It will be understood that while a poly-ß-amino-ester is a product of a Michael Addition of an amine with an acrylate, methacrylate or similar conjugated carboxylic acceptors other reactions such as concomitant amide formation may also occur.

In one embodiment, the present application provides a method for preparing a microcapsule, the method comprising: (a) preparing an oil-in-water emulsion of (i) an oil phase comprising at least one multifunctional amine donor with at least one multifunctional acceptor, and at least one lipophilic core; and (ii) a water phase comprising at least one stabilizer, at least one defoamer, or at least one emulsifier, (b) optionally adding at least one catalyst, at least one diluent or at least one initiator to the oil phase; (c) heating the oil-in-water emulsion with stirring to a temperature between 25° C. and 100° C. and so forming the polymeric microcapsule shell by an in-situ oil-in-water reaction of the amine donor(s) with the acceptor component(s); and (d) obtaining the lipophilic core encapsulated in a polymeric microcapsule shell.

Thus, there are described below non-limiting generic routes or embodiments for the practical application and implementation of the polymeric shell microcapsules of the invention which are able to encapsulate and retain lipophilic cargoes including aggressive cargo examples such as fragrances or volatile oils, while concomitantly such polymeric shells also being biodegradable according to the criteria described herein, and which are made via processes which, at the encapsulation stage avoid the need for high temperatures, and/or avoid the need to use or remove volatile or otherwise undesirable solvents or reagents. Some of these processes involve in-situ polymerizations wherein all reactants donors and acceptors are in the same oil phase at the outset and wherein for each of the process route variants specific modifying reactants or components are used which introduce specific features into the poly-ß-amino ester capsule shells, such features selected from introduction of hydrophobic or sterically hindered or rigid or charged moieties, or moieties able to be stable in or respond to pH extremes or pH changes (‘pH responsive’) such that the poly-ß-amino ester polymeric shell is able to achieve a desired balance of biodegradability—storage stability in aqueous media or formulated products—and a triggered cargo release. These routes include as generic descriptions: (i) Pre-modified amine (or prepolymer) route (ii) Post-modified reaction route and (iii) selective mixed monomeric reactants route.

(i) Pre-modified amine (precursor), or prepolymer route: A poly-ß-amino ester capsule shell is formed via an in-situ polymerization process wherein one or more multi-functional amine donors are reactively pre-modified, or partially polymerized into a prepolymer or reactive oligomer before the oil in water emulsion formation and prior to the encapsulation shell formation. It is recognized that if these types of resultant modified amine molecules were available, they could be used directly in the encapsulation stage. Such pre-reactive modifications are designed to specifically introduce hydrophobic or sterically hindered or rigid or charged or pH responsive moieties into the poly-ß-amino ester polymeric shell. Further they are progressed to a stage that still allows dissolution in the oil phase before the emulsification and oil-in-water encapsulation reaction stage.

In another embodiment, the present application provides a method comprising: (a) pre-reacting at least one multifunctional amine donor with a modifying reagent to form a modified amine donor; (b) preparing an oil-in-water emulsion of (i) an oil phase comprising at least one amine functional donor including at least one modified amine donor as prepared in (a), with at least one acceptor(s), and at least one lipophilic core; and (ii) a water phase comprising at least one stabilizer, at least one defoamer or at least one emulsifier, (c) optionally adding at least one catalyst, at least one diluent or at least one initiator to the oil phase; (d) heating the oil-in-water emulsion with stirring to a temperature between 25° C. and 100° C. to form the polymeric microcapsule shell by an in-situ oil-in-water reaction of the amine donor(s) with the acceptor component(s); and (e) obtaining the lipophilic core encapsulated in a polymeric microcapsule shell.

In this generic route, firstly, a prepolymer or a pre-reactively modified monomeric reactant precursor (modified multifunctional amine) is synthesized which has residual reactive functional amine (primary and/or secondary NH) groups—either as end groups and/or distributed along the chain or molecule. At least two residual N—H groups, on average, are present on the modified amine donor. Such prepolymer or modified amine donor also contains, either covalent or ionically bonded or complexed, hydrophobic or sterically hindered or rigid or charged groups, or groups able to be stable in pH extremes or pH changes (‘pH responsive’). This prepolymer or modified amine donor is then used in a subsequent microencapsulation process, which preferably, though not essentially, will be a sequential process in the same vessel as the prepolymer/modified amine donor synthesis, or it can be performed later in a new vessel. In these cases microencapsulation proceeds via dissolution of the prepolymer or pre-modified amine donor in the cargo, with any other reactants, optionally with added diluent, (altogether forming an oil or organic phase), typically with warming, followed by emulsification of that oil phase with an aqueous phase, followed by a reaction of the functional groups (donors and acceptor) that are present in the prepolymer or modified amine donor with added co-reactive acceptor reagents which form chain extensions, and/or branches and/or crosslinks during the oil-in-water-encapsulation. The modified amine donor may be prepared via a pre modifying reaction which may be ‘neat’ or may be in the presence of any of the eventual oil phase components, or portions of them.

The diluent will preferably be a liquid at room temperature or readily meltable at moderate temperatures such as below 90° C. or less than 50° C. and may be a hydrocarbon oil, an alkane, a melted wax, an ester oil, a fatty acid ester, an aliphatic ester, or an alkylene carbonate. Some specific examples include mineral oil, long chain alkanes such as hexadecane and the like, aliphatic esters such as esters of long chain acids such as caprylates, myristates, oleates, cocoates, palmitates, or stearates including isopropyl myristate as one example, or long chain esters of shorter chain acids or other monohydric or polyhydric esters.

As an example, a multifunctional amine such as PEHA is modified via a pre-reaction with a mono functional acrylate or methacrylate at a stoichiometry which leaves multiple N—H groups still available for further reaction. Any polyfunctional amine may be used in these ways. Other modification reactions can be used as described.

(ii) Post-modified reaction route: A poly-ß-amino ester capsule shell is formed via an in-situ oil-in-water polymerization process wherein one or more multi-functional amine donors are used in stoichiometric excess when forming the initial oil-in-water emulsion and completing an initial encapsulation shell formation, followed by a post encapsulation modifying reaction of the unreacted (excess) amine groups, such post-reactive modifications designed to specifically introduce hydrophobic or sterically hindered or rigid or charged groups, or groups able to be stable in pH extremes or pH changes (‘pH responsive’) into the poly-ß-amino-ester polymeric shell.

In another embodiment, the present application provides a method for preparing a microcapsule, the method comprising: (a) preparing an oil-in-water emulsion of (i) an oil phase comprising at least one amine functional donor with at least one acceptor, and at least one lipophilic core; and (ii) a water phase comprising at least one stabilizer, at least one defoamer or at least one emulsifier, (b) optionally adding at least one catalyst, at least one diluent or at least one initiator to the oil phase; (c) heating the oil-in-water emulsion with stirring to a temperature between 25° C. and 100° C. and so forming the polymeric microcapsule shell by an in-situ oil-in-water reaction of the amine donor(s) with the acceptor component(s); (e) obtaining the lipophilic core encapsulated in a polymeric microcapsule shell, and (f) effecting a post-modification reaction on the polymeric shell material to add hydrophobic, sterically hindered, charged, rigid or pH responsive functional groups to the shell material.

The donor or acceptor reactant comprises a hydroxyl group reactive functionality incorporated into the polymeric shell or polymeric shell comprising poly-ß-amino ester and is used for the subsequent post reactive modification step, to add hydrophobic, sterically hindered, charged, rigid or pH responsive functional groups to the shell material. The hydroxyl functionality is co-introduced via (i) inclusion of hydroxyethyl acrylate, hydroxyethyl methacrylate, hydroxypropyl acrylate, hydroxypropyl methacrylate, hydroxybutyl acrylate, hydroxybutyl methacrylate, or via (ii) inclusion of hydroxyl functional amines selected from ethanolamine, diethanolamine, amino pentanol, amino butanol, and aminopropanol. The total donor or the total acceptor reactants are used in stoichiometric excess to leave unreacted amine or acceptor functional groups available for subsequent post-reactive modification.

Typically, the post-reaction is a further (in one (same) pot) Michael Addition of added mono-acrylate or mono methacrylate or other mono functional acceptor which introduces hydrophobicity, sterically hindered groups, charge or pH response groups, via react with the stoichiometric excess of amine NH groups. Other post modification reactions using the stoichiometric excess of NH groups can be applied. For example, reactions with epoxides, isocyanates, acids, anhydrides, acid chlorides, esters, aldehydes or ketones or halogenated hydrocarbons can be used in pre- or post-modification reactions.

The pre-modification or post-modification reaction can thus be via a Michael Addition Reaction of a selected donor and acceptor combination. Non-limiting examples of a mono-functional reactant that can participate in a modifying pre- or post-reaction that is a Michael addition, are acceptors selected from the group consisting of: (i) acrylamido sulfonic acid (AMPS), acrylic acid, methacrylic acid, carboxyethyl acrylate, dimethylaminoethyl acrylate or methacrylate (DMAEA/DMAEMA), tert-butyl amino ethyl acrylate or methacrylate (TBAEA/TBAEMA), (ii) cationically charged mono functional acrylate or methacrylate selected from trimethyl aminoethyl methacrylate chloride ((MBJ), 3-Acrylamidopropyl) trimethyl ammonium chloride (APTAC), 3-methacrylamido propyl trimethyl ammonium chloride (MAPTAC), trimethylamine ethyl acrylate chloride (MBS), (iii) an acryloyl- or methacryloyl-phosphate, (iv) lauryl, n-alkyl or branched alkyl acrylate or methacrylate, sec-alkyl acrylate or methacrylate, tert-butyl acrylate or methacrylate, tert-alkyl acrylate or methacrylate, isobornyl, menthyl, cyclohexyl; and (v) cycloaliphatic mono-acrylate, or mono-methacrylate, or is phenoxyethyl, benzyl, or any phenyl ring containing mono-acrylate or mono-methacrylate.

The pre-modification or post-modification reaction can be a Michael Addition Reaction of a methacrylate acceptor; wherein, the shell formation reaction is a subsequent Michael Addition of an acrylate acceptor(s); and wherein, the final shell material comprises both ß-aminoethyl ester groups, and ß-amino-(1-methyl-ethyl) ester groups.

Alternatively, the pre-modification or post-modification reaction to introduce the desired functional groups can be progressed via:
(i) reaction of excess or unreacted amine groups with monofunctional acid, monofunctional acid derivative, difunctional acid, difunctional acid derivative, half ester of difunctional acid, or with acid chloride, or acid anhydride, and derivatives thereof, so forming amides, or with a monofunctional or difunctional epoxide, isocyanate, halocarbon or aldehyde or ketone; or via
(ii) reaction of an available or added hydroxyl group with monofunctional acid, difunctional acid, half ester of difunctional acid, or with acid chloride, acid anhydride, and derivatives thereof, so forming esters, or with a monofunctional or difunctional epoxide, halocarbon, isocyanate, aldehyde or ketone; or via
(iii) reaction with an amino acid.

As one example, glycidyl trimethylammonium chloride and related reagents (containing epoxide groups and ammonium groups) can introduce charged (cationic) moieties into the pre-reacted (modified) amine donor, or post-reacted poly ß-amino-ester shell. Long chain acid chlorides such as lauroyl chloride can introduce hydrophobic moieties into a pre-reacted (modified) amine donor, or in to a post-reacted poly ß-amino-ester shell.

Non-limiting examples of acid or acid derivatives for post-modification reaction are selected from the group consisting of C₂-C₂₀ alkanoic acids, propanedioic acid, butanedioic acid, hexanedioic acid, octanedioic acid, decanedioic acid, sebacic acid, dodecanedioic acid, dodecenylsuccinic acid, octenyl succinic acid, cholesteric acid, itaconic acid, maleic acid, fumaric acid, and malonic acid.

(iii) Mixed Monomeric Reactants Route:

A poly-ß-amino ester capsule shell is formed via an in-situ polymerization process wherein a selected mixture of one or more multi-functional amine donors, optionally with mono functional amine donors with one or more multifunctional acceptors, optionally with mono functional acceptors and are used when forming the oil in water emulsion and the encapsulation shell formation and wherein such mixed monomeric reactants (donors and/or acceptors) are designed to specifically introduce hydrophobic or sterically hindered or rigid or charged groups, or groups able to be stable in pH extremes or pH changes (‘pH responsive’) moieties into the poly-ß-amino ester polymeric shell.

The polymer shell as made by any of the process variants of the present application may also contain unsaturated groups at a chain end or distributed along the chain and the in-situ reaction to form the polymeric shell may include reaction of the unsaturated groups via (i) a chain extension, (ii) branching or (iii) crosslinking reaction either during the polymeric shell formation or subsequent to it.

Where a prepolymer or a polymer shell formed from monomeric reactants or other routes contains excess or residual unsaturated reactive groups, an additional crosslinking mechanism may be applied and in particular a radical based crosslinking of the reactive groups. For this a (one or more) radical initiator, which may be a peroxide or an azo based radical initiator or a redox system such as a persulfate based system, or which may be a photo-initiator for UV induced radical reactions can be added at any stage and a radical reaction linking unsaturated groups is initiated and progressed. This approach (added radical initiators and radical polymerization) may also be used to ‘mop up’ or reduce free monomeric unsaturated molecules if present. Initiators can be in either the oil and/or the water phase. In addition, this approach can facilitate the (free radical) linking (or crosslinking) of saccharides or proteins or other molecules with the poly-ß-amino-ester and such saccharides or proteins may be bear, or be, the modifying hydrophobic, sterically crowded, pH responsive or charged moiety. Radical linking reactions between the poly-ß-amino ester (which could be made with free acrylate groups attached) to OSA (octenyl succinic acid) modified, or DSA (dodecenyl succinic acid) modified, saccharides, or similarly hydrophobically modified saccharides, sugar alcohols, polyols or proteins are coupled or linked. OSA starch is one example of such a molecule. Other examples include fatty acid derivatives, or alkyl- or alkenyl-acid derivatives, of other saccharides, sugar alcohols (such as sorbitol or xylitol), polyols or of proteins.

In all the methods described above, the water phase or oil phase, or where both phases, comprises a radical initiator system selected from peroxide based, an azo based, a redox based, or comprises a radical chain transfer agent, added at any point of the process. These methods employ a water soluble or an oil soluble monofunctional Michael acceptor. These processes incorporate a radical addition or polymerization reaction to add crosslinking or to consume residual acceptor or donor functionality, wherein such reaction is performed at a temperature ≤130° C. or ≤100° C. The reaction can also be a crosslinking reaction performed at a temperature of ≤130° C. or at a temperature of ≤100° C. or of ≤30° C. using glutaraldehyde glyoxal, isocyanates, acid, acid derivatives, or epoxides. the oil in water emulsion is prepared with or without the application of heat. The catalyst can be employed in all the described methods and is a tertiary amine.

When designing capsule shells for the most demanding of applications, for example encapsulation of fragrances for liquid fabric conditioner products, a higher crosslink density is typically required and/or some other form of rigidity and ‘solvent/chemical resistance’ or resistance to the more extreme pH's (as is sometimes experienced in formulated end products), in the shell polymer structure. This typically translates to an ability to achieve a noticeable fragrance boost (bloom) or release upon physical crushing or via other triggers, considered highly advantageous for such products. Furthermore, such capsules are of course also required to remain ‘intact’ as capsules with fragrance inside (note fragrance is an ‘aggressive solvating or plasticising cargo’ compared many others) and retained inside for relatively long time periods, until such a crushing or other triggered release in use (by consumers) occurs. More particularly they are also often required to be stable (‘intact’) when stored before ultimate consumer end-use in formulated products which might be of extreme pH's such as pH3 and/or long time periods and/or contain solvents or ingredients that might compromise the polymer shell wall. As described above on the prior art examples of capsule technologies reported to meet such demanding needs are melamine-formaldehydes (M-Fs) and crosslinked acrylates.

These form durable capsule shells with long term storage stability in formulated aqueous media such as pH3 often also containing surfactants, as is the case for some liquid fabric conditioner products, and all in the presence of the ‘aggressive’ cargo (fragrance). However, as also described above in the prior art, these M-F or acrylate or related capsules are not biodegradable in aquatic media such as seawater, river/surface water or activated sludge, nor are they compostable according to recognized international standards (such as EN/ISO, ASTM, OECD etc.). Furthermore, such highly crosslinked capsule shells are not readily made to be biodegradable while retaining performance (fragrance boosts) or storage stability. Our invention has discovered routes to make stable capsules (e.g. stable on storage until use)—so resisting the solvating or plasticising/softening effect from the inside (fragrance cargo) and resisting the effects of the formulation components which may be at an aggressive pH3 and/or contain a surfactant mix (from the ‘outside’ formulation medium), but which will also show biodegradation or evidence of non-persistence in water based media (aquatic systems) and still perform, for example as a fragrance booster (when fragrance is the cargo) when triggered. Thus, in the case of the more demanding circumstances, where a high crosslink density capsule shell is likely required to retain a cargo, stably, on storage in such situations, the products of the processes described, and compositions described in this invention can achieve that.

In another embodiment, it is disclosed that the microcapsule is stable as a core shell capsule in an aqueous slurry, in a water-based formulation or in a solvent-based formulation. The microcapsule is storage stable as a core shell capsule in solid formulated or printed product. Accordingly, the formulation or aging medium has pH in the range of 3-4, 4-5, 5-6, 6-7, 7-8, 8-9, 9-10 or 10-11, or 11-12. The inventive microcapsule is stable as a core-shell capsule in an aqueous slurry or in a water-based formulation having pH in the range 3-5. The inventive microcapsule is stable as a core-shell capsule in an aqueous slurry or in a water-based formulation having pH in the range 9-11.

In another embodiment, microcapsules are used in in home care (laundry products, cleaning products), personal care (hair, skin, oral products) and industrial sectors (such as coatings, adhesives, agricultural products, energy markets) and others. As such many different formulations or use environments are encountered.

In another embodiment, the microcapsules of the present invention are formulated into a laundry detergent, fabric softener, fabric conditioner, shampoo, hair conditioner, liquid soap, solid soap, skin deodorant, skin moisturizer, skin conditioner, hair or skin protectant, cleanser, sanitizer, cleaning fluid, dishwashing fluid, dishwashing tablet, washing powder, washing tablet, washing liquid, and cosmetic formulation.

In a specific embodiment, the microcapsule is used in a fabric conditioner composition or a laundry detergent composition.

The capsules of this invention which are biodegradable or non-persistent in aquatic tests and which show encapsulation can perform and be stable in many formulations, including water-based formulations or solutions at various pH's and with various additives present including surfactants or salts, and also in solvent based formulations or products and also in dry or waterless or low water content products (tablets, larger capsules, powders or powder blends, gels). In making or formulating such products, the capsules of the invention can be directly incorporated as a slurry as is produced by the process of production or may be added as a dried product (e.g. the capsules may be spray dried or freeze dried or fluid bed dried or dried by any other drying process, to make dried capsules). Examples are given below of spray drying for example to make a free-flowing powder or to make an over-coated capsule.

For addressing the most demanding of requirements in terms of stability in some formulations, or in solvents, or in water-based formulations away from neutral pH, one possible route, as described above, would be to make higher crosslink density polymers. However, it is well known by those skilled in the art that conventional covalent crosslinking will typically slow down or inhibit biodegradation processes.

We have surprisingly found that some particular highly crosslinked structures (as capsule shells) which have achieved a higher cross link density can show a combination of being robust capsules (‘bloom’ performance and storage stability in aqueous formulations) can still be hydrolysable or biodegradable or show evidence of non-persistence over time when tested for biodegradability in aquatic media. The use of hydrolysable or cleavable crosslinks, in some cases, can surprisingly lead to achieving a combination of robust capsules, stable on storage in water-based formulations, including pH extremes, yet which can show biodegradability in aquatic environments or test media.

Such materials showing evidence of biodegradability in aquatic environments will also be compostable.

For fragrance encapsulations via the in-situ polymerization route, it is not convenient to make crosslinked networks via reactions that need high temperatures (e.g. condensation reactions between acids and alcohols)—this is undesirable if encapsulating a volatile or reactive cargo such as a fragrance, in-situ. Michael addition reactions are another known route to achieve crosslinking at moderate temperatures. Typically, an amine (primary or secondary) donor reacts, under mild conditions, with an acceptor, typically a molecule with a conjugated double bond such as an acrylate, methacrylate, acrylamides, methacrylamides, itaconates, maleates, fumarates, or maleimides, among others. Poly-ß-amino-esters (Aza Michael reaction) are formed.

However, we have further discovered that a polymer shell or a copolymeric polymer shell structure which contains ß-amino-ester moieties, in a crosslinked network, can be designed to have enhanced stability in pH's away from neutral compared to analogous or known polyamino-ester polymers per se, and that through selection of the suitable specific multifunctional and/or mono-functional donor-acceptor combinations via specific processes to make them, robust capsules can be made for encapsulating fragrance or other cargoes which are also, at the same time, storage stable, including at pH extremes or in formulated products, and also able to be biodegradable or show evidence of non-persistence in aquatic or other environments or media.

Furthermore, we have discovered that such an approach can surprisingly achieve high crosslink densities in making capsules which are required for a boost or bloom performance (triggered fragrance release) in some applications, and also for storage stability in aqueous formulations that might be considered aggressive (destabilizing) in their pH and/or surfactant use] and yet still also be, over time, biodegradable or non-persistent in aquatic media after use, as evidenced by OECD or other tests for example. Such stable (on storage) crosslinked capsule structures for the most demanding of encapsulations are able to be formed by the use of specific multi-functional reactants in the in-situ Michael reaction—wherein the reactants (donor and acceptors) selected for, or designed for (for example by reactive modifications), introduction of hydrophobic or sterically hindered or rigid or charged or pH responsive groups into the shell stricture, and wherein these so formed capsule shells are still biodegradable or non-persistent in aquatic media.

In terms of achieving sufficiently crosslinked materials for some applications, for a combination of storage stability in relatively aggressive (e.g. away for neutral pH optionally with surfactants and/or salts present) water based media, and a fragrance bloom in terms of performance as well as maintaining biodegradability after use or non-persistence over longer time periods, the required crosslinking can be achieved by for example using at least, in a part of the system, one functional reactant with a functionality of 3 or more, so designated as A3+B2, A3+B3 or A3+B4 etc. Other (lower functionality such as mon-functional) reactants may also be present in addition. For applications requiring the more robust shells preferably at least one of the multifunctional reactants will have reactive functionality of three or more, preferably four or more. More preferably both of the multifunctional reactants will have a reactive functionality of three or more or four or more—in order to achieve capsules which can produce a fragrance bloom, and which are stable in fabric conditioner and similar low pH media. Preferably such reactants will be chosen to, or will have been made via pre-reactions to, introduce hydrophobic or sterically hindered or rigid or charged or pH responsive groups into the shell stricture. Alternatively, or in addition, such features will have been introduced via post-reactive modification of the initial polymer shell.

We have thus discovered that the drawbacks associated with known poly-ß-amino ester capsule shells such as sensitivity to, or storage sensitivity to, a more strongly plasticizing cargo while in an aggressive formulated end product at a pH extreme such as 3 or 11, while still retaining biodegradability or potential for non-persistence according to criteria described herein, can be overcome by the alternative approach of in-situ Michael Addition oil in water polymerization using selected combinations of donor and acceptors or using pre-modified donor and/or acceptor reactants, and/or via post-reactive modifications of the capsule shells to introduce into robust, typically crosslinked, capsules a balance of storage stability in formulations while retaining the potential for biodegradability.

Herein described as one embodiment of our invention is an in-situ oil in water encapsulation via a Michael addition polymerization in the oil phase with a selected combinations of donors and acceptors, some of which may have been pre-modified.

Herein described as another embodiment of our invention is an in-situ oil in water encapsulation via a Michael addition polymerization in the oil phase with a selected combinations of donors and acceptors, wherein the initial capsule shell formed is selectively further reacted or modified. Such approaches have the advantage of allowing for the selection of more lipophilic or hydrophobic donor and acceptor monomers and including monomers (acceptors and donors] with higher functionalities, which in turn reduces the risk of excess residual monomer and ensures that high crosslink densities and suitably retentive and stable capsules are obtained or can be more readily tailored. Furthermore, early gelation is limited due to dilution (solvation) in the cargo (oils, fragrances or other hydrophobic cargoes).

In another embodiment, the present polymeric microcapsule shell is biodegradable in an aquatic medium or solid medium or is compostable. The aquatic or solid medium is selected from group consisting of activated sludge, secondary effluent, river water, surface water, fresh water, sea water, soil and compost.

In another embodiment, the polymeric microcapsule shell material shows a biodegradation rate of at least 20% in an aquatic medium when measured by an OECD Test method 301, 302 or 306. The polymeric microcapsule shell material shows evidence of biodegradation within 120 days or within 60 days or within 40 days or within 28 days.

In another embodiment, the microcapsule is storage stable as a core-shell capsule in an aqueous slurry, in a water-based formulation or in a solvent-based formulation. The microcapsule is storage stable as a core-shell capsule in a solid, largely waterless formulation or in a printed product.

In another embodiment, the present application provides a microcapsule showing a retained triggered release of cargo or ‘a bloom’ after storing or aging in respective medium for at least 4 weeks at ambient temperature (15-25° C.), or at least 6 weeks or at least 8 weeks or at least 12 weeks at ambient temperature.

In another embodiment, the present application provides a microcapsule showing a retained triggered release of cargo or ‘a bloom’ after storing or accelerated aging in respective medium for at least 2 weeks, for at least 3 weeks, for at least 4 weeks, for at least 6 weeks, for least 8 weeks, fir at least 10 weeks or for at least 12 weeks at an elevated temperature of 40° C. Accordingly, the microcapsule shows a retained triggered release of cargo or ‘a bloom’ after storing or aging in a liquid laundry detergent formulation of acidic pH for at least 4 weeks at ambient temperature or 40° C., or at least 6 weeks or at least 8 weeks or at least 12 weeks at ambient temperature or 40° C.

For the most demanding aqueous media for storage or delivery of the capsules such as pH 2 or 3 or pH 11 or 12 higher crosslink densities are preferred and yet surprisingly the microcapsules made can be biodegradable or non-persistent according to OECD or other standard tests.

For many monomer combinations, an in-situ oil in water polymerization can lead to a core-shell morphology, and if sufficiently highly crosslinked (higher functionality donors and acceptors), lead to robust capsules for good fragrance retention, and/or good long-term stability and/or bloom (burst) of fragrance release following deposition on to fabric, including cotton swatches for tests, and breakage through friction, for example.

For the in-situ polymerization process amines (and other donors, and also acceptors) which are of a lower water solubility, compared to for example common multifunctional aliphatic amines, often highly water soluble are among the donors that are preferentially used in some embodiments to achieve a balance of biodegradability—storage stability and applications performance attributes. However, limited choice exists in available polyfunctional amines for this and hence we have adopted selected reactive modifications or selected combinations of the monomeric reactants. Nevertheless, we have surprisingly found that when polyfunctional amines including water soluble amines, are distributed into the oil phase and the polymerization-encapsulation effected in one (the oil) phase with no significant reactants necessarily in the water phase, with our reactive modifications or combinations good performance robust microcapsules. Some polyfunctional amines are available which have relatively lower water solubility or hydrophilicity or affinity and which can be present in the oil phase (where the cargo and other reactant are also present in the processes of our invention). Tetramethylpiperidine (TMPP) is one example of a lower water soluble multifunctional amine. However, it is difunctional only so alone will have some limitations in targeting high crosslink densities if used alone. While there are other amines which do not substantially partition into the water phase when distributed into the oil phase, or which may be considered as relatively hydrophobic, they tend not to be, typically, aliphatic amines with the higher amine functionalities—which are typically more hydrophilic. However, if multifunctional amines are suitably pre-reacted or modified, their hydrophobicity can be higher and/or their tendency to partition out can be reduced. Additionally, such modified multifunctional amines can lead to more storage stable (in aqueous or formulated product media). Selectively, reactively pre-modified polyfunctional amines can achieve this. Similarly, selective combinations of amines can achieve this in a poly-ß-amino-ester. Aromatic amines can be use in the process though are sometimes associated with less desirable toxicity profiles in handling—though they can be used in all the processes of the invention and will have some applications.

In-situ polymerizations where all reactants are in one phase (here, the oil phase)—may have some potential issues or limitations if water soluble reactants or reactants with high affinity for water are to be included. While such monomeric reactants can be readily used (either applied in the water phase or applied to the pre-made emulsion of the oil phase and water phase) for interfacial polymerizations, when a highly water soluble monomer (donor or acceptor) when such highly water soluble monomeric reactants are used in an in-situ oil in water polymerization, where all reactants are in the oil phase from the start (or prior to the reaction starting), there is potential for it to partition out into the water phase and so not fully participate in the in-situ polymerization (which takes place in the oil phase). In some cases that may limit the network structure and limit formation of a more robust shell. This can lead to non-formation of core shell capsules or of soft capsules, or a partial collapse of the capsule structures or it may lead to a more open (loose) structure or a matrix type of capsule structure, typically less well performing in terms of robust cargo retention or of storage stability.

We have discovered a route to overcome this and use in-situ oil in water polymerization successfully to make robust microcapsules, and where, initially, a more water soluble (potentially partially partitioning) donor (or acceptor) molecule is desirable to use, for example to control a biodegradation profile, and so incorporate it into the capsule shell structure. Thus, in another embodiment any amine, including hydrophilic amines, can be incorporated into the capsule shell polymer by an approach whereby the amine donor is pre-reacted, in bulk or with an oil carrier or diluent present, via a Michael Addition with a monofunctional or polyfunctional acceptor (e.g. monofunctional or multifunctional acrylate, methacrylate, acrylamide, methacrylamide, itaconate, fumarate, maleimide or other known Michael Acceptors such as ketones) such that some of the NH bonds pre-reacted via the pre-reaction with the acceptor molecules to form a more hydrophobic precursor which may then be considered as a modified monomeric reactant or a prepolymer. This so forms, essentially, a new donor in-situ which contains the amine derived moiety within it, and which has residual unreacted N—H groups and so can be further reacted in a subsequent Michael Addition reaction (the in-situ polymerization stage, where cargo is present along with the remaining monomeric reactant (s)). This can all be done sequentially in the same reaction vessel if desired. The in-situ polymerization stage then uses the modified monomeric reactant or precursor prepolymer based on a pre-modified multifunctional amine, with residual unreacted N—H, and this is reacted, optionally with other added donors and wherein preferentially the overall stoichiometry is largely matched with the double bond acceptor groups present from the multi-functional acceptor(s).

In this way, by doing such a pre-reaction, which can also be a Michael Addition, a polymer of a poly-amino ester formed in part at least from a pre-modified multi-functional amine with residual unreacted N—H groups, is able to be produced via a subsequent in-situ oil in water polymerization with all reactants in the oil phase, with any amine, even water soluble ones, and this can be beneficial to tune biodegradability performance and capsule performance in terms of fragrance release bloom testing and microcapsule storage stability even in pH's away from neutral. A pre-reaction (in bulk or solvent) of a water-soluble polyfunctional amine can be incorporated as a first step [separate or integrated (one-pot)] in the process] as a precursor step in the overall process—to make it less hydrophilic. A similar effect can also be achieved, for amine donors, by making a precursor amide or oligomeric amide with residual unreacted amine (NH) groups (made via excess amine functionality in a reaction with a mono- or di functional or multi-functional acid or acid chloride or anhydride or ester)—so making a precursor adduct wherein the part of the amine is reacted or slightly chain extended via amide formations to make a less water soluble or less hydrophilic molecule containing residual unreacted amine moieties, so creating a new donor with amide bonds present and free amine (NH) groups retained (for subsequent reactions in the subsequent in-situ polymerization phase).

Other pre-reactions of a multi-functional amine—with mono- or multi-functional reagents which co-react with amines, performed at less than stoichiometric equivalence (so as to retain unreacted N—H groups to subsequently form poly-ß-amino-ester polymeric shells in an in-situ oil-in-water polymerization encapsulation reaction) can be performed to make other pre-modified polyfunctional amines. This allows polyfunctional amines (desired for achieving a desired crosslink density) that may have a high affinity for water (often the case for polyfunctional aliphatic amines) to then be used effectively in a subsequent (after pre modification of the polyfunctional amine) oil-in-water in-situ polymerization (encapsulation) process (with all reactants in the oil phase). Examples of other pre-reactions (or post-reactions) to modify the capsule shell after initial formation) useful to modify a polyfunctional amine, leaving residual unreacted N—H groups for subsequent encapsulation reactions, include reactions with mono-functional or di-functional isocyanates, epoxides, acids, acid chlorides, acid anhydrides, esters, halogenated hydrocarbons, aldehydes or ketones, higher functionalities of such modifying reagents may also be used, in part at least, provided that the pre-modified reaction products with residual unreacted amine (NH) groups can be solubilized in the oil phase (all reactants plus cargo (e.g. fragrance and/or oil and/or diluent)) of the subsequent in-situ polymerization-encapsulation.

While introducing hydrophobicity or a reduced affinity for water, in the capsule shell structure and/or in the process for capsules preparation is one route to make more storage stable poly-ß-amino-ester capsules it is having been discovered that other targeted modifications are also able to do this. For example, introduction of charge or pH responsiveness into the poly-ß-amino ester is another route either alone or in conjunction with a hydrophobic modification.

Without being bound by theory it is suggested that a more stable (improved storage stability in aqueous and formulated product media) biodegradable shell can be made using, in part at least, pre-modified polyfunctional amines or selected combinations of amines—with particular acceptors or mixtures of acceptors. Often using (unmodified) hydrophilic polyfunctional amines, while useful in some applications requiring shorter term release as in some biotechnology or biomedical applications (where they have largely been used to date) will lead to relatively poor stability on storage in aqueous or formulated products such as those envisaged for the products of the invention, which tend to have pH's above or below neutral, for example acidic pH3 and this can accelerate degradation (hydrolysis) of the poly ß-amino esters—so making them too unstable for use in formulated laundry or consumer care products for example. Using suitably pre-modified polyfunctional amines or suitable selected mixtures of amins, as donors or by suitable post-modified capsule shells one can facilitate tailoring the required balance of biodegradability-storage stability—triggered release (bloom) performance and such approaches can be designed to improve storage stability (in aqueous or formulated products). Suitable modifications or introductions are those introducing, into the poly ß-amino ester structure hydrophobic or sterically hindered or rigid or charged groups, or groups able to be stable in pH extremes or pH changes (‘pH responsive’).

Non-limiting examples of modifying acrylate or methacrylates (other acceptors with similar characteristics can also be used in the pre-modification reaction) are those introducing structure hydrophobic or sterically hindered or rigid or charged groups, or groups able to be stable in pH extremes or pH changes (‘pH responsive’), and include: mono-functional acrylates or methacrylates such as isopropyl-, propyl-, ethyl-, methyl-, butyl-, pentyl-, hexyl-, ethyl-hexyl-, tert-butyl-, iso-octyl-, nonyl-, iso-decyl-, lauryl-, stearyl-, behenyl-, iso bornyl-, norbornyl-, cyclohexyl-, menthyl-, benzyl-, phenoxy ethyl-, or charged or pH responsive, acrylates and methacrylates. Examples of modifying charged mono-functional acrylates or methacrylates, or acrylates or methacrylates that can accommodate pH extremes (high/low; pH responsive) by changes to form charged molecules or complexes (e.g. amine functional, carboxylic acid functional) include—acrylic acid, methacrylic acid, ß-carboxyethyl-acrylate/methacrylate and its oligomers, trimethyl amino ethyl acrylate chloride (MBS), (3-Acrylamidopropyl) trimethyl ammonium chloride (APTAC), Trimethyl aminoethyl methacrylate chloride (MBJ), (3-methacrylamidopropyl) trimethyl ammonium chloride (MAPTAC), 2-Acrylamido-2-methylpropane sulfonic acid (AMPS), tert-butyl aminoethyl-acrylate/methacrylate (TBAEA/TBAEMA), dimethylamine ethyl acrylate/methacrylate (DMAEA, DMAEMA).

It will be understood that in some cases reacting or modifying molecules can be used which may not be charged at the start but can bear a charge—so although not charged at the point of starting the reaction later they can become charged by pH adjustments or via addition of reagents. An example would be a tertiary (or other amine) amine group which can then bear a charge via formation of quaternary ammonium salts. Addition of salts or alkylation reactions with alkyl halides or alkyl sulfates, or alky carbonates, or other known methylation or alkylation agents to make quaternary ammonium salts. Examples of reagents to transform a tertiary (or other) amine, into an ammonium or quaternary ammonium salt would be reaction (of amine) with certain alkyl halides to form ammonium or quaternary ammonium chlorides and bromides (for example). Reactions with alkyl sulfates (dimethyl sulfate for example) or methane sulfonic acid or other sulfonic acids can make ammonium or quaternary sulfates. Dimethyl carbonate and similar reagents can also make quaternary ammonium products (carbonates). Tertiary amines can be present in the multifunctional amine donor(s) or in the acceptor(s) or in both when the poly ß-amino esters are formed and can be subsequently modified into ammonium or quaternary salts by such reactions. Such charged or pH responsive moieties can also be present in starting monomeric reactants that form the initial poly-ß-amino-ester.

It will also be understood that other known or designed hydrophobic, rigid or sterically hindered or charged or pH responsive functional acrylates or methacrylates can be used. By way of an example mono-acrylates, -methacrylates, -acrylamides, methacrylamides, or -itaconates are preferably used to pre modify highly polyfunctional amines (e.g. PEHA, TEPA) in bulk or with added cargo and/or diluent, and the modified amine is then reacted with PETA or Di-PETA and the like in the subsequent oil-in water encapsulation stage. Di- and tri-functional acrylates, methacrylates, acrylamides, methacrylamides or itaconates can be used in certain cases in a pre-modifying reaction, alone or admixed with mono-functional acceptors, provided that the stoichiometry of the initial reaction is such that the product (which is an intermediate for the subsequent encapsulation reaction) is dissolvable in the oil phase for the subsequent in-situ polymerization encapsulation. Similar pre-modifications of a polyfunctional amine donor can be achieved with hydrophobic, sterically hindered, rigid or charged or pH responsive, or similarly functional reactants that will react with amines. These reactive modifying agents would include epoxides, isocyanates, acids, acid chlorides, acid anhydrides, esters, halogenated hydrocarbons, and aldehydes or ketones. Mono-functional modifications at or around 1 or 2 mole eq. to the N—H groups on a polyfunctional amine are preferred, or between 0.5 and 2 mole eq. or 0-5-1 mole eq. of the modifier reagent reactive functionality to the amine (NH). Higher levels can be accommodated especially in amines with higher initial multifunctionality. In all cases, sufficient unreacted N—H groups will need to remain after the formation of modified amine or prepolymer intermediate to allow subsequent reaction to form the microcapsule shell, via reaction with multifunctional Michael Addition acceptors and the modified amine intermediate will need to be soluble in the oil phase prior to homogenization/subsequent reaction, such oil phase containing the cargo, the remainder of reactants (other acceptors—and donors if present) and optionally a diluent noting that some of these may be present already from the pre-modification reaction.

Similar effects can be achieved via a targeted post reaction (modification after an initial encapsulation reaction, that is after a first stage oil-in-water in-situ polymerization). In such cases the first reaction—all reactants (and cargo or oil/diluent solubilized cargo) in the oil phase—is carried out with an excess of amine NH functionality such that after an initial shell formation, during an in-situ Michael addition polymerization with multifunctional acrylate or other acceptor. Then there is an addition of added modifying reagent, (e.g. a mono functional acrylate or methacrylate, or other modifying reactant as described above for the pre-reaction modification), to react with residual (unreacted free)N—H groups. Reactive modifying agents include epoxides, isocyanates, acids, acid chlorides, acid anhydrides, esters, halogenated hydrocarbons, and aldehydes or ketones. Mono-functional modifications at or around 1 or 2 mol eqs to the N—H groups on a polyfunctional amine are preferred, or between 0.5 and 2 mol eqs or 0-5-1 mol eq, of the modifier reagent reactive functionality to the amine NH. Higher levels can be accommodated especially in amines with higher initial multifunctionality. Modifying reactants which introduce hydrophobicity, steric hindrance, charge or functional groups responsive to pH changes, as described above, are preferred. Such post-modifications can impart improvements in storage stability (in aqueous or formulated products) and/or bloom performance of the microcapsules.

Examples of post shell modification reactions can be facilitated by an excess of amine donors or acceptors in the initial shell formation stage. In both cases second stage Michael Additions with added reagents can be used as described above. However, those ‘free’ functional groups (amine and/or conjugated double bonds from acceptors) can be reacted with other modifying molecules such as, in the case of free amines, with acids, anhydrides or acid chlorides, epoxides isocyanates, halocarbons or aldehydes which can react with excess amines on the initial formed shell to introduce the modifying functions or groups of the invention. For free (excess) conjugated double bonds, reactive modifications via radical polymerizations can be used and this is also a route to introduce additional linking or crosslinking, or for residual monomer consumption at the end of the reaction. In the case of the latter radical initiator and/or chain transfer agents are useful for mopping up residual (unreacted) monomers and achieving very low residual monomer levels. Oil soluble or water soluble radical initiators or redox initiators may be used. In the case of modifying reactions using mono-functional modifying acrylates, methacrylates acrylamides, methacrylamides and other acceptors or indeed any residual multi-functional reactants, such radical reactions can reduce residual monomer levels. Where modifying reactants for the invention are water soluble or form water soluble polymers, as is often the case with the charged or pH responsive modifiers for example, any residual (unreacted or ‘free’) amounts can be mopped up or chased by radical initiators or chain transfer agents and so would form, typically, polymers which are water soluble (present in minor or trace amounts) and can remain without the need for removal and without interference or drawbacks and can also in some applications add benefits in performance or stability in formulations. They may also participate in coacervation processes.

In some examples of the invention donors and/or acceptors which have additional functionality suited to subsequent reactive modifications after an initial shell formation can be used as a vehicle to introduce the desired modifications of hydrophobicity, steric hindrance, charge or pH responsiveness. Examples include hydroxy functional amines (amino butanol, amino pentanol, diamino pentanol and hydroxyl bearing amines or polyfunctional amines, or hydroxyl functional acrylates or methacrylates (or other acceptors) for example hydroxyethyl acrylate/methacrylate, hydroxypropyl acrylate/methacrylate, hydroxybutyl acrylate/methacrylate. Such functional amine donors or acceptors allow for reactions of the hydroxyl group after initial shell formation (or before if so desired) to introduce those features of the invention into the poly ß-amino ester shell such as via esterification reactions of the hydroxyl with acids or acid anhydrides or acid chlorides with hydrophobic, sterically hindered, charged or pH responsive functional groups. In the case of using acid anhydrides for modifying free hydroxyl (esterification reactions) or free amine groups (amidation reactions) hydrophobicity and charge can be introduced via use of alkenyl succinic anhydrides for example octenyl succinic anhydride or dodecenyl succinic anhydride which once reacted have hydrophobic chains plus also generate a free carboxylic acid which can be used for other reactions or interactions and can be used as a pH responsive or charged group.

In the use of the Michael addition reaction, similarly a mixture of different amine(s) as donors can also be used advantageously to tailor a balance between biodegradability and encapsulation performance or stability on storage, including in pH's at or away from neutral such as 3 or 11, and including in formulated products such as liquid fabric conditioners/softeners, shampoos, soaps, deodorants, skin creams, insect repellent delivery, cleaning fluids, sanitizers, agricultural active delivery, among others. Thus, similar effects as described above can be achieved in some cases via a targeted mix of modifying reactants (e.g. monofunctional acrylates/methacrylates—or others as described above) altogether at the start of the process—and then crosslinking (multifunctional) reactants all present together from the start of the reaction. In such cases all reactants (and cargo or oil/diluent solubilized cargo) are in the oil phase at the outset and the reaction is carried out at or near stoichiometric equivalence (of all the co-reacting functional groups). Thus, preferably this would be a mono-functional acrylate or methacrylate which introduces hydrophobicity, steric hindrance, charge, or functional groups responsive to pH changes, as described above, are preferred within a mixture of multifunctional acrylate(s) and multifunctional amine(s), such as PEHA—all reacted via an in-situ Michael addition polymerization. Such combinations can impart improvements in storage stability (in aqueous or formulated products) and/or bloom performance of the microcapsules.

Again, other co-reactive modifying reagents could be accommodated in a mixture of multifunctional amine donors and multifunctional acrylate (or other) acceptors—and selected to introduce the same desired features to the amine or shell polymer. These reactive modifying components include epoxides, isocyanates, acids, acid chlorides, acid anhydrides, esters, halogenated hydrocarbons, and aldehydes or ketones, and as such introduce parallel and/or sequential reactions to the Michael Additions. They are selected for their ability to introduce hydrophobicity, steric hindrance or charge or pH responsiveness. Mono-functional reagents at or around 1 or 2 mole eq·s to the N—H groups on a polyfunctional amine are preferred, or between 0.5 and 2 mole eq·s or 0-5-1 mol eq, of the modifier reagent reactive functionality to the amine NH. Higher levels can be accommodated especially in amines with higher initial multifunctionality.

A lower overall multi-functionality (as determined by the overall total multifunctionality of the system), from all multifunctional donors and acceptors in the polymerization, and the presence of an increased amount of monofunctional reagents, can result in relatively lower crosslink densities would likely result in a more biodegradable shell other things being equal and can be used, in conjunction with the pre-modified amine donor, to tailor the required balance of biodegradability-storagestability-triggered release (bloom) performance.

In most cases overall stoichiometry (donor-acceptor groups, in the final polymeric product) is preferably largely maintained at or around 1:1 in terms of overall donor and overall acceptor reactive (functional) group equivalents. Variations can be accommodated, small or large, though smaller variations are preferred and for example within about 30, 20 or 10 mol equivalent % or within 5 mol equivalent % or within 1 mol equivalent %.

In another embodiment, the present application provides a method for preparing microcapsules comprising a polymeric microcapsule shell based on ß-amino ester functionalities, the method comprising: a) pre-reacting a multifunctional amine or mixtures thereof with a monofunctional or difunctional or multifunctional acrylate or methacrylate or itaconate, as acceptor or mixtures thereof, such that the reaction product remains soluble in the oil phase of (b) and has residual amine NH functionality (so adjusting stoichiometries to ensure polyfunctionality in NH's remain); b) preparing an oil-in-water emulsion of (i) an oil phase comprising the product of (a) and one or more acceptors selected from acrylate-, methacrylate-, acrylamide-, methacrylamide, or itaconate functional molecules, optionally mixed with a difunctional or multi-functional amine and at least one lipophilic core, optionally with a diluent; and (ii) a water phase comprising at least one stabilizer or emulsifier, optionally adding at least one catalyst to the oil phase or water phase; c) forming the polymeric microcapsule shell wall by an in-situ oil-in-water Michael addition polymerization reaction of the donor and acceptor reactants, and d) obtaining the core encapsulated in a polymeric microcapsule shell.

Optionally the microcapsule shell may also contain an added polymer. As such the polymeric microcapsule shell may further comprise added zein, other protein, a polypeptide, or a polymer having hydrophobic or sterically hindered or charged or pH responsive functional groups. The polymer may be added at any stage of the process as a powder, dispersion or can be solubilized in one of the phases or in one of the components of the phases. For example, zein or other polymer can be added as a pre-dissolved solution in an amine donor such as PEHA.

Although an in-situ polymerization is a preferred route for some of the embodiments, it is to be noted that other emulsion polymerizations including classical interfacial polymerization methods and its variations, including process variations as described above (pre-reactions, post-reactions selected mixtures of monomeric reactants) can also be used to introduce specific modifications as described that tis hydrophobic, sterically hindered, charged or pH responsive moieties. Such features can be introduced into the microcapsule shell structures via interfacial polymerizations, following similar approaches and using the same or similar modifying reagents, as described for the in-situ processes above. in-situ processes are preferred and have advantages over other routes.

It will also be understood that the capsules of the invention, and through any of the embodiments or variations, can be dried or made into coated or double layered capsules via that route. This can enhance storage stability further and/or performance further. The double layered, multilayered or over coated microcapsule comprises a hydrogel or a crosslinked alginate. Examples of such concepts are described further below. Microcapsules of the present application have an average diameter of about 100 nm to 100 μm though distributions can span outside of this range and capsules can be made larger if desired. More typically average particle size ranges from about 1 μm to 100 μm. By varying reaction conditions and relative concentrations, particle sizes can be varied. All examples below fall within these ranges.

One embodiment encompasses the use of coacervates, or components of a coacervate forming system, as a stabilizer or as an additive which is used as a route to introduce a secondary outer layer or coating to the microcapsules described above. The use of charged moieties in the shell, introduced for example by selection of monomeric reactants, or via pre- or post-reactive modifications as described above, can facilitate this further, no is not an essential requirement in all cases. Moieties able to accept or donate a charge, or which are pH responsive are also useful components in the shell polymer to facilitate such interactions. Examples are described below but as a general description of one approach:

A cationic polymer for example a cationic polysaccharide such as cationic guar is dissolved in the aqueous phase. A stabilizer such as polyvinyl alcohol may be present as well as other additives for example a defoamer if required. The aqueous phase is mixed with the oil phase (which contains the cargo and all reactants) and the mixture stirred and homogenized. The encapsulation reaction is then progressed, and at any point during this or after completion of shell formation an anionic polymer, for example an anionic polysaccharide such as xanthan gum is then added. The orders of addition can be reversed for the anionic and cationic components. Furthermore, either of the components could be introduced at other points of the process whether at the start or during the encapsulation reaction or process or after the shell wall completion. Other cationic and anion polymer combinations can be used (to facilitate coacervate formation). Anionic polysaccharides (carboxymethylcellulose, xanthan gum, gum arabic, carrageenan, alginic acid/alginate, pectin), or cationic polysaccharides such action cationic-guars, or -gums or -dextran, or cationic surfactants are preferred. These coacervates, present as outer or secondary coatings can also be crosslinked for example by aldehydes such as glutaraldehyde or glyoxal. Such crosslinking may also encompass crosslinking of the poly ß-amino esters if there are suitable reactive moieties available, which include amine or hydroxyl group among others.

Other routes to applying outer coatings are also available. For example, slurries of capsules of the invention as made by processes described can be encapsulated in a second coating of crosslinked sodium alginate. In such an approach one way of demonstrating that is to filter the microcapsules as made and disperse into a buffered solution of sodium alginate in water. That mixture can be then be added slowly with stirring (via an addition funnel or syringe) into a stirred solution of calcium chloride, which crosslinks the alginate around the capsules, so forming an outer or secondary coating. Larger capsules than the original (‘visible beads’) were formed which were composed of the microcapsules of the invention surrounded or embedded in a crosslinked alginate coating or overlayer. Other crosslinked outer coatings can be similarly applied with acid functional polysaccharides and calcium or other di- or multi-valent chlorides or bases. Other crosslinking reactions and approaches as may be required. This includes hydrogels from polysaccharides for example.

Another route to applying an outer coating is via complexation or coacervate formation optionally followed by crosslinking. Poly-ß-amino ester shell materials generally, through their amine environments, can in some circumstances form complexes or coacervates under certain conditions, which may involve pH adjustment for optimizations for example, with added anionic molecules or polymers. The poly-ß-amino esters have tertiary amine environments when forming the ß-amino-ester links through reactions of secondary amines and may also have secondary amine environments if only reacted once of primary amines or if left residual or may in some cases have primary amines which may not have reacted. Such moieties can form complexes or coacervates with added anionic polymers or molecules and may form the basis of an outer coating with or without crosslinking. Such coated microcapsules may be formed in-situ as slurries and may also be spray dried to produce solid coated microcapsules which may then be used as is, or via redispersion into an aqueous slurry. When additional functional groups such as described are also present in the polymeric shell the complexation or coacervation may in some cases be enhanced.

Poly-ß-amino ester shell materials of the invention which bear tertiary amine or quaternary ammonium or charged or acidic or pH responsive moieties can also participate in coacervate formation and subsequent crosslinking, this aiding the formation of an outer coating. Poly-ß-amino ester shell materials of the invention can be prepared such they contain one or more moieties of acid (R—COOH/—COO—; R—SO₃H/—SO₃—for example) and ammonium (quaternary, tertiary and other ammonium moieties (NR4+, or NR₃H+) or tertiary amines (NR₃) or residual secondary or primary amines, which can all show pH responsiveness and aid stabilities in some pH extremes. Such functional poly-ß-amino esters, bearing charges or bearing groups able to accept or donate charges or protons, can be used as a component or contributor or enhancer in coacervation processes as described above for introducing secondary coatings, or their presence in the shell polymer composition can strengthen those outer coating interactions based on a coacervation approach. They may also be designed to be crosslinkable for example via the use of aldehydes.

Poly-ß-amino esters bearing charges or bearing groups able to accept or donate charges or protons such as those just described may be used in another embodiment of the invention based on a coacervation process as the route to the initial polymer shell formation (rather than forming or contributing to a secondary coating). Poly-ß-amino esters with free amine groups or bearing ammonium groups (cationic) as described in other embodiments can form coacervates with anionic polymers such as anionic polysaccharides as the basis for a polymeric capsule shell with a lipophilic cargo. Poly-ß-amino esters as described in other embodiments can also bear anionic groups (carboxylic acid; sulfonic acid) and when both cationic or anionic moieties, or groups capable of forming cationic or anionic moieties, are present in the same polymer structure or are present in two different poly-ß-amino esters suitably admixed, coacervation processes can progress and can form capsule shells. Crosslinking of such shells can be facilitated through reactions, after initial shell coacervate formation via reactions of suitable crosslinking chosen depending on what might be residual or available, or designed into in such pol-ß-amino esters and any other coacervate forming partner polymers. For example, crosslinking may be via aldehydes (glyoxal, glutaraldehyde) or via enzymatic processes (transglutaminase) or via free radical groups if unsaturated groups are present or residual in the poly ß-amino ester shell structure or any other coacervate forming polymer also present. Other crosslinking approaches such as added multi-valent metals/ions, epoxy/glycidyl ether, acid anhydride and others known to those skilled in the art may be used depending on what functional groups are residual or designed in.

Thus, in one embodiment of the invention a pre-made poly ß-amino ester bearing cationic groups (or groups able to form cationic groups) is suitably mixed at a chosen pH with an anionic polysaccharide or a poly-ß-amino-ester, or other polymer bearing anionic groups (or groups able to form anionic groups)—and wherein one of these components contains the cargo, optionally with diluent, all pre-dissolved in. Optionally a crosslinking agent is included in one component and stabilizers or other additives. By adjustment of pH and/or heat a coacervate is formed around the cargo, and the coacervate shell which can be subsequently crosslinked. In this approach or embodiment, the poly-ß-amino ester, and other coacervate forming polymers, can be linear or branched polymers and so can be made from di-valent amine donors and divalent acceptors—as well as with multifunction donors or acceptors of higher functionalities, as may be desired.

In some examples of the invention a microcapsule with a lipophilic core and a biodegradable polymeric shell is demonstrated by: (a) making an oil-in-water emulsion of an oil phase which comprises all donor and acceptor reactants and a cargo, optionally with added diluent or solvent and/or aided by application of heat, and a water phase containing a stabilizer or emulsifier, optionally with other additives, (b) optionally adding a catalyst or initiator to one phase (c) forming the polymeric capsule shell wall by an in-situ oil-in-water addition polymerization reaction of the donor and acceptor reactants; and (d) obtaining the cargo encapsulated in a polymeric microcapsule shell.

The diluent will preferably be a water immiscible liquid at room temperature or readily meltable at moderate temperatures such as below 90° C. or less than 50° C. and may be a hydrocarbon oil, an alkane, a melted wax, an ester oil, a fatty acid ester, an aliphatic ester, or an alkylene carbonate. Some specific examples include mineral oil, long chain alkanes such as hexadecane and the like, aliphatic esters such as esters of long chain acids such as caprylates, myristates, oleates, cocoates, palmitates, or stearates including isopropyl myristate as one example, or long chain esters of shorter chain acids or other monohydric or polyhydric esters.

The catalyst is preferably a base added to the oil phase, such as triethylamine or another tertiary amine.

When pre-reacting amine donors first in a Michael Addition Polymerization-Encapsulation in some circumstances it has been found that amines can be more stably or readily incorporated into a hybrid poly-ß-amino ester shell by firstly reacting or capping one or more of the amine groups with acceptor molecules, as a first step, in bulk or with added fragrance and/or diluent carrier, via a pre-reaction with all or a portion of the acceptor (e.g. acrylate or methacrylate), optionally aided by heating. This is particularly useful in the case where the amine is more water soluble and so not well suited to an in-situ oil in water polymerization wherein all reactants are to be in the oil phase. Such pre-end-capping or pre-reacting of amine donors can result in lower water soluble donors. If low stoichiometries and there is residual free NH— then they can serve as modified donors as described herein. Alternatively, the stoichiometry of the pre-reaction can be such that substantial or near whole transformation of N—H groups to make adducts or oligomeric derivatives, or, collectively, prepolymers of the amine with the mono- or di- or multi-functional acrylate or other acceptor, and so all or most of the amine NH's now bear acrylate bonds (where NH bonds were previously), rendering them considerably more lipophilic or less hydrophilic compared to the starting amine itself. Such adducts or initial prepolymer or oligomeric products remain as relatively low molecular weight adducts which may remain soluble in cargo and/or added diluent and/or added excess acrylate, or which can be otherwise readily solubilized, optionally aided by heat. The encapsulation stage of this pre-reaction route variant is progressed with homogenization/dispersion and added further donor and/or acceptor molecules at or near a stoichiometrical equivalence (or approximately so) in terms of available acrylate groups remaining after reactive modifications of some of the donor amine groups).

Further, certain aspects of the present application are illustrated in detail by way of the following examples. The examples are given herein for illustration of the application and are not intended to be limiting thereof. Figures show optical microscopy images of examples of microcapsules made using various polymers and via various processes described. Figures show sensory test results for fragrance release from microcapsules prepared via the various processes described. Figures show biodegradation data of microcapsule shell materials prepared by various processes described.

Thus, in the range of process variants and/or compositional variations, embodiments, descriptions and examples of the microcapsules of the invention it will be understood that they are able to be used for many types of lipophilic cargoes and in many media or applications (formulated end products, including waterless or solid format products or solvent based products or formulations or in neutral or near neutral pH aqueous formulation media) and do perform in delivering some fragrances and/or other cargoes more readily encapsulated or retained and/or stored, while also showing biodegradability or non-persistence.

In another embodiment, the present application provides a polymeric microcapsule shell biodegradable in an aquatic medium or solid medium or is compostable. The aquatic or solid medium is selected from group consisting of activated sludge, secondary effluent, river water, surface water, fresh water, sea water, soil, and compost.

In another embodiment, the polymeric microcapsule shell material shows a biodegradation rate of at least 20% in an aquatic medium when measured by an OECD Test method 301, 302 or 306. The polymeric microcapsule shell material shows evidence of biodegradation within 120 days or within 60 days or within 40 days or within 28 days.

In another embodiment, the microcapsule is storage stable as a core-shell capsule in an aqueous slurry, in a water-based formulation or in a solvent-based formulation. The microcapsule is storage stable as a core-shell capsule in a solid, largely waterless formulation or in a printed product.

In another embodiment, the present application provides a microcapsule showing a retained triggered release of cargo or ‘a bloom’ after storing or aging in respective medium for at least 4 weeks at ambient temperature (15-25° C.), or at least 6 weeks or at least 8 weeks or at least 12 weeks at ambient temperature.

In another embodiment, the present application provides a microcapsule showing a retained triggered release of cargo or ‘a bloom’ after storing or accelerated aging in respective medium for at least 2 weeks, for at least 3 weeks, for at least 4 weeks, for at least 6 weeks, for least 8 weeks, for at least 10 weeks or for at least 12 weeks at an elevated temperature of 40° C. Accordingly, the microcapsule shows a retained triggered release of cargo or ‘a bloom’ after storing or aging in a liquid laundry detergent formulation of acidic pH for at least 4 weeks at ambient temperature or 40° C., or for at least 6 weeks or for at least 8 weeks or for at least 12 weeks at ambient temperature or 40° C.

In another embodiment, the present application provides a double layered microcapsule, a multi-layered microcapsule or an overcoated microcapsule. Accordingly, the double layered, multilayered or an overcoated microcapsule comprises within its outer coating a polysaccharide, a protein, a hydrogel, a coacervate or is a polymer bearing hydrophobic, charged, pH responsive groups or formulations of polymers comprising one or more such polymers. Accordingly, the double layered, multilayered or an overcoated microcapsule comprises within its outer coating a xanthan gum, a polysaccharide gum, an alginate polymer, a cellulose ether including hydroxyethyl cellulose or carboxymethyl cellulose, a guar or modified guar including cationic guar, zein protein, a protein, a hydrogel, a coacervate or a polymer bearing hydrophobic, charged or pH responsive groups.

In another embodiment, the present application provides inventive microcapsule having an average diameter of about 100 nm to 150 μm or about 1 μm to 100 μm.

The following examples illustrate the present disclosure, parts and percentages being by weight, unless otherwise indicated. Each example is provided by way of explanation of the present disclosure, not limitation of the present disclosure. In fact, it will be apparent to those skilled in the art that various modifications and variations can be made in the present disclosure without departing from the scope or spirit of the invention. For instance, features illustrated or described as part of one embodiment, can be used on another embodiment to yield a still further embodiment. Thus, it is intended that the present disclosure covers such modifications and variations as come within the scope of the appended claims and their equivalents.

### EXAMPLES

In-Situ Michael Addition Polymerization, with Selected Amine Donors and/or Selected Acceptors or Specific Mixtures Thereof Introducing Hydrophobic or Steric Hindered or Charged or pH Responsive Moieties into the Polymeric Capsule Shell—without any Pre- or Post-Modifications

This example illustrates the in-situ polymerization process which can be applied to make poly-ß-amino-ester microcapsules of the invention which are good quality, and which are stable in some formulated products including solid or waterless or printed products, as well as some aqueous formulated products.

### Example 1

Preparation of Microcapsules Having Polymer Shell Comprising a Poly-ß-Amino-Ester Made from 1 Mol·Eq. Dipentaerythritol Penta/Hexaacrylate (Di-PETA, Acrylate Functionality, f, 5.7) and 1 Mol·Eq. Pentaethylene Hexamine (N—H Functionality f, 8) and Encapsulation of Home Care Fragrance Sunburst Fresh R14-3913 (220-31-2).

An aqueous phase was prepared by mixing 26.02 g of 10% aqueous solution of polyvinyl alcohol and 163.24 g of deionized water. 0.16 g of defoamer was also added. An acrylate oil phase was prepared by dissolving 7.21 g (13.7 mmol, f 5.7; total acrylate mmol:78.1) of dipentaerythritol penta/hexaacrylate (Di-PETA) in 30 g Fragrance Sunburst fresh. An amine oil phase was prepared by dissolving 2.28 g (9.8 mmol, f8; total NH mmol: 78.4)) of penta-ethylene hexamine in 12.35 g of Fragrance Sunburst fresh. 8.47 g of diluent, propylene glycol dicaprylate/caprate, was added followed by 0.28 g of Triethylamine (catalyst).

The amine oil phase was added to the acrylate oil phase under mechanical stirring to form the internal phase. The internal phase was then added to the aqueous phase under mechanical stirring to form a coarse emulsion. The coarse emulsion was then homogenized using an IKA magic lab homogenizer, 1 pass at 4000 rpm. The formed emulsion was transferred to a reactor pot and the emulsion was heated to 35° C. The oil-in-water emulsion was then left to react for 24 hours to complete polymerization. The resulting microcapsule slurry was an aqueous slurry of microcapsules which were visible under a light microscope and which released fragrance upon crushing and exhibited a satisfactory bloom (noticeable triggered release of fragrance). Optical micrographs are shown in FIG. 1 for 220-31-2.

The same procedure as above (with all reactants in the oil phase—in-situ polymerization) was applied to other amines such as TMPP (4,4-trimethylenepiperidine), isophorone diamine (IPDA) and hexamethylene diamine (HMDA) as illustrative examples of the range of amines able to be used, and also mixtures of amines. In all cases microcapsules were clearly formed and exhibited an initial release of fragrance up on crushing.

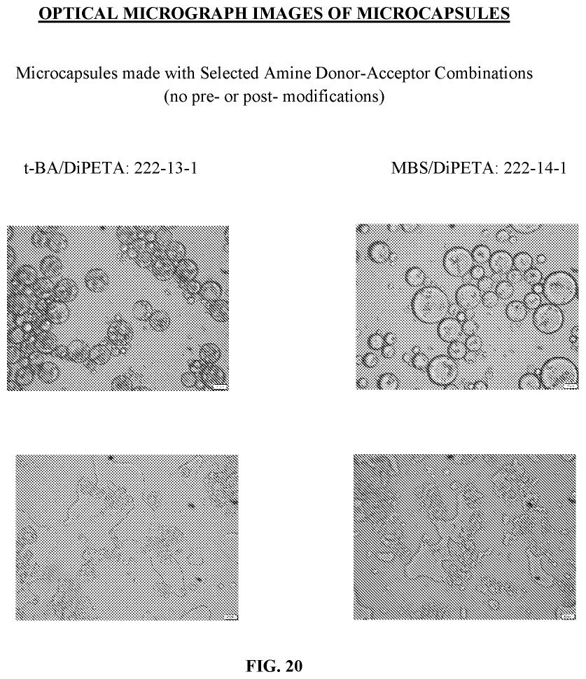

The same procedure as above was also applied to further specific examples of amines or specific mixtures of amines with specific acceptors or mixtures of acceptors where one or more of the monomeric reactant components bear hydrophobic, sterically hindered, charged or pH responsive groups. For example, monofunctional acrylates containing such groups are added into the di-PETA, as an example of a multifunctional acrylate acceptor, and the mixture used as the acceptor component as describes above for di-PETA alone, to react with PEHA in the same procedure as in the Example above. Similarly, charged or pH responsive or hindered, or hydrophobic amines are used in addition to (mixed with) a multifunctional amine such as PEHA as the donor component. The overall stoichiometry is such that polymer and most preferably crosslinked polymer is formed. Examples included a mixture of DiPETA and trimethyl amino ethyl acrylate chloride (MBS: Ref 222-14-1) and a mixture of tertiary-butyl acrylate (t-BA)-DiPETA (222-13-1) as the acceptor components in reactions with PEHA as described above. The overall acrylate: N—H stoichiometry was about 1:1 and mono-functional acrylate was present at such a loading that, on average, 1-2 N—H groups on PEHA were targeted for reaction with the monofunctional component, and this introduces or adds more, hydrophobicity, or charge or pH responsiveness. Other ratios are feasible provided a sufficient ratio or number of N—H groups can react with the multifunctional acceptor components for polymer shell formation. The resulting microcapsule slurry was an aqueous slurry of microcapsules which were visible under a light microscope and which released fragrance upon crushing. Optical micrographs are shown in the Figures (FIG. 20 for 222-13-1 and 222-14-1).

Examples of the Invention Via the Prepolymer/Reactive Pre-Modification Route with Michael Addition Reaction:

Preparation of Microcapsules having polymer shells comprising of 1 mol·eq Dipentaerythritol penta/hexa-acrylate (functionality of 5.7 on acrylate) and modified PEHA wherein 1 or 2 mol·eq of NH's on PEHA were pre-reacted with mono-acrylate or mono-methacrylate or other mono-functional acceptors to make a modified PEHA (either, leaving unreacted, on average, N—H (average) functionalities of 6 or 7: reduced from 8 and depending on whether 1 or 2 mol eq of mono-functional pre-modifier was used)—and designed so (approximately) a matched (1:1) stoichiometry overall of the two co-reactive groups was attained. Other ratios of overall stoichiometry are also able to be used.

### Example 2: Using a Hydrophobically Pre-Modified Amine

Preparation of Microcapsules Having Polymer Shell Comprising of 1 Mol. Eq. Lauryl Methacrylate Modified Pentaethylene Hexamine (220-61-1) and Encapsulation of Home Care Fragrance Sunburst Fresh R14-3913

A modified amine was prepared by heating 9.55 g (41.1 mmol; f8; total NH 329 mmol) penta-ethylene hexamine to 60° C. 10.45 g (41.0 mmol; f1) lauryl methacrylate was added under mechanical stirring. The modified amine was then left to react for 3 hours to complete modification. An aqueous phase was prepared by mixing 26.02 g of 10% aqueous solution of polyvinyl alcohol and 162.61 g of deionized water. 0.16 g of defoamer was also added. An acrylate oil phase was prepared by dissolving dipentaerythritol penta/hexaacrylate (5.40 g; 10.3 mmol; f5.7; total acrylate f+58.7 mmol) in 20 g Fragrance Sunburst fresh. An amine oil phase was prepared by dissolving the modified PEHA (Lauryl Methacrylate modified Penta-ethylene hexamine (ave f7; ave MW 486.79) (4.08 g; 8.38 mmol; 58.7 mmol amine NH) in 12.35 g of Fragrance Sunburst fresh. 8.47 g of propylene glycol dicaprylate/caprate (Waglinol) was added followed by 0.30 g of triethylamine. The amine oil phase was added to the acrylate oil phase under mechanical stirring to form the internal phase. This internal phase was added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 35° C. The oil-in-water emulsion was then left to react for 24 hours to complete polymerization. The resulting microcapsule slurry was an aqueous slurry of microcapsules which were visible under a light microscope.

### Example 3: Using a Pre-Modified Amine Bearing Charge or pH Responsive Groups

Preparation of capsules from [2-(Methacryloyloxy) ethyl]trimethylammonium chloride (also known as Trimethyl aminoethyl methacrylate chloride, MBJ; ref capsules reference: (220-62-1) as modifying agent introducing charge to the multifunctional amine, pentaethylene hexamine (PEHA) and encapsulation of home care fragrance Sunburst fresh R14-3913 by subsequent reaction with dipentaerythritol penta/hexa-acrylate.

A modified amine was prepared by: (i) heating Pentaethylene hexamine (PEHA; 10.56 g 45.4 mmol) to 60° C.; (ii) adding [2-(methacryloyloxy) ethyl]trimethylammonium chloride (MBJ; 12.59 g (75%; 9.44 g, 45.4 mmol)) with mechanical stirring; (iii) stirring the two reactants for 3 hours at 60° C. to complete a reactive modification of a portion of the amine NH-s of PEHA, so introducing a charged group onto some of the amine NH's of PEHA. The stoichiometry of the two reactants is 1:1 molar which leads to, on average, 1 of the 8 NHs of PEHA reacted with 1 monofunctional methacrylate. Product average MW is 440.08 and average functionality on residual NH is 7.

An aqueous phase was then prepared by mixing 26.02 g of 10% aqueous solution of Polyvinyl alcohol and 162.61 g of deionized water. 0.16 g of defoamer was also added.

An acrylate oil phase was prepared by dissolving 5.64 g (10.8 mmol; f5.7; 61.6 mmol acrylate groups) dipentaerythritol penta/hexaacrylate in 20 g Fragrance Sunburst fresh. A modified amine oil phase was prepared by dissolving the modified amine (4.46 g (86.4%; 3.85 g; 8.75 mmol f7; 61.3 mmol NH) ([2-(methacryloyloxy) ethyl]trimethylammonium chloride modified Pentaethylene hexamine) in 12.35 g of Fragrance Sunburst fresh. 8.47 g of Propylene glycol dicaprylate/caprate was added followed by 0.30 g of triethylamine.

The amine oil phase was added to the acrylate oil phase with mechanical stirring to form the final internal phase. This internal phase was added to the aqueous phase with mechanical stirring to form a coarse emulsion. The coarse emulsion was homogenized using IKA magic lab homogenizer, 1 pass at 4000 rpm. The formed emulsion was transferred to a reactor pot and the emulsion was heated to 35° C. The oil-in-water emulsion was then left to react for 24 hours to complete polymerization. The resulting microcapsule slurry was an aqueous slurry of microcapsules which were visible under a light microscope.

The same procedures as above with a pre-modification reaction of a mono-methacrylate modified multifunctional amine, followed by an oil-in-water (in-situ polymerization) encapsulation reaction (with all reactants in the oil phase prior to homogenization and in-situ polymerization) was applied to PEHA with other modifying monofunctional methacrylates at 1:1 molar (mono-methacrylate: PEHA so targeting, on average, reactive modification of 1 N—H on PEHA) and also, for some examples, at 2:1 molar (mono-methacrylate: PEHA, so targeting, on average, reactive modification of 2 N—H's on PEHA). Typical modifying methacrylates used with PEHA amine (or other amines) were used such that on average either 1 NH or 2 NH's were modified from the starting 8 amine NH's (4 secondary; 4 primary), other ratios are also usable including average substitutions on NH's of less than 1, between 1 and 2 and over 2. It is expected the primary NH's would be preferentially modified first.

Examples of mono-methacrylates or methacrylamides (also able to be used as acceptor) used to make microcapsules in this way included: tert-butyl methacrylate (t-BMA, 220-58-1), lauryl methacrylate (LMA, 220-61-1—as described above), stearyl methacrylate (SMA, 220-60-1), trimethyl amino ethyl methacrylate chloride (MBJ, 220-62-1 as described above) methacrylamide-propyl-trimethylammonium chloride (MAPTAC, 220-63-1).

For each modifying acceptor, either an average of 1 NH group of the PEHA (most probably mostly primary NH) reacting with 1 methacrylate was targeted, or pre-reaction stoichiometries were targeted for reaction with (on average) 2 NH groups on PEHA (most probably both the primary NH's—making capsules with references ending in ‘2″ rather than ‘1’, other numbers reaming the same). All approaches and examples made microcapsules which exhibited clear release of fragrance upon crushing. Figures below show examples. Evaluations in bloom tests and aging tests are shown in tables below.

A similar process was also applied to pre-modification reactions of a multifunctional, using PEHA as an example, using mono-functional acrylates or acrylamides. In these cases, rather than heating to 60° C., the pre-modifying reactions were carried out at ambient temperatures (20-35° C.) for about 4 hours with all other details the same as above. The second stage with multifunctional (di-PETA) acrylate proceeded for 20-24 hours at 35° C. Example modifying mono-acrylates used in such reactions, so introducing into the poly-ß-amino ester capsule shell hydrophobicity steric hindrance, charged or pH responsive moieties were: butyl acrylate (BA, 220-40-2) tert-butyl acrylate (t-BA, 220-57-1) benzyl acrylate (BzAc, 220-43-1 and -2 (with a double loading of modifier)), isobornyl acrylate (IBA, 220-44-1), stearyl acrylate (SA, 220-42-1 and -2), trimethyl amino ethyl acrylate chloride (MBS, 220-45-1; and 220-45-2 with a double loading of MBS to target an average reaction stoichiometry of 2 NH groups), 3-acrylamidopropyl trimethyl ammonium chloride (APTAC, 220-46-1), 2-Acrylamido-2-methyl-1-propanesulfonic acid (AMPS, 220-05-1), Acrylic acid (AA, 220-08-1) ß-carboxyethyl acrylate (CEA, 220-07-1), oligomeric ß-carboxyethyl acrylate (CEAO, 220-06-1)).

Similarly, the same procedure of modifying the amine donor was used to prepare microcapsules with a tetrafunctional and trifunctional acrylate with a pre-reaction of PEHA with MBJ, a charged pH responsive moiety, (targeting MBJ pre-reaction with 1 NH on PEHA, on average) and then polymerizing, with capsules formation, with pentaerythritol triacrylate (PETA-TRI, with MBJ pre mod: 220-89-1) and pentaerythritol tetra-acrylate (PETA-TETRA with MBJ pre mod: 220-88-1). Microcapsules were also prepared without a pre-modification with these lower functionality acrylates (following Example 1 procedure). They were: 220-86-1: PETA-TETRA/PEHA (1:1 mol eq on reactive groups) and 220-87-1: PETA-TRI/PEHA (1:1 mol eq on reactive groups). Other stoichiometry ratios are able to be used.

Other reactions can also be applied to pre-modify the amine donor (or post modify a polymer shell). One example is the reaction with lauroyl chloride which introduces an amide group with hydrophobicity into the amine (and shell). The method for that is described below:

PEHA (reactor charge, 9.70 g) is pre-heated in a reactor to 40° C. The pH of the reactor charge is adjusted to 9.5 by addition of 1M sodium hydroxide. Lauroyl chloride (feed; 10.3 g) is added dropwise (slowly) to the reactor. The mixture is then reacted for 3 hours maintaining the pH around 9.5 and then allowed to cool to ambient temperature. The reaction product, with formation of an amide modified PEHA with hydrophobic group, was neutralized with 1M hydrochloride acid. A portion of this product (3.91 g, 60.67 mmol eq of amine NH estimated on average) is then reacted with Di-PETA penta/hexa-acrylate (5.58 g; 60.64 mmol eq of acrylate functionality estimated) in an oil in water encapsulation reaction.

The modified PEHA amine is dissolved in about half of the fragrance cargo (R1439-13 Green Woody) in a beaker while the remaining fragrance with diluent (Waglinol) and triethylamine catalyst, are mixed in another beaker. In parallel an aqueous phase was prepared comprising water, POVAL (polyvinyl alcohol) and Agitan 295 (defoamer), in another beaker. The final oil phase was formed by mixing: the amine-fragrance mix was added into the fragrance-diluent-catalyst mix and the two mixed briefly. This was then promptly added to the aqueous phase—the whole was the stirred for about a minute and then homogenized at 4000 rpm into a reaction flask. The reaction was carried out with stirring for 24 hrs at 35° C. during which microcapsules (ref 220-55-1) were clearly formed and which showed fragrance release and bloom upon crushing.

### Examples of Crosslinked Poly ß-Amino-Ester Microcapsules by In-Situ Oil-In-Water Addition Polymerization with Post Reactive Modification

### Example 4

### (Post-Modified Polymer Shell Using Monofunctional Charged Acrylate or Methacrylate and Excess of Polyfunctional Amine):

Preparation of Microcapsules Having Polymer Shell Comprising of 1 Mol·Eq Dipentaerythritol Penta/Hexaacrylate, 1.1 Mol·Eq Pentaethylene Hexamine and 0.1 Mol·Eq [2-(Methacryloyloxy)Ethyl]Trimethylammonium Chloride and Encapsulation of Home Care Fragrance Sunburst Fresh R14-3913

An aqueous phase was prepared by mixing 26.02 g of 10% aqueous solution of Polyvinyl alcohol and 162.91 g of deionized water. 0.16 g of defoamer was also added.

An acrylate oil phase was prepared by dissolving 7.27 g Dipentaerythritol penta/hexaacrylate in 20 g Fragrance Sunburst fresh. An amine oil phase was prepared by dissolving 2.53 g of pentaethylene hexamine in 12.35 g of Fragrance Sunburst fresh. 8.47 g of propylene glycol dicaprylate/caprate was added followed by 0.29 g of triethylamine.

The amine oil phase was added to the acrylate oil phase under mechanical stirring to form the internal phase. This internal phase was added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 35° C. The oil-in-water emulsion was then left to react for 20 hours to complete polymerization.

The excess amine post modification was carried out by adding 2.38 g (75%) 2-(methacryloyloxy)ethyl]trimethylammonium chloride to the microcapsule slurry under mechanical stirring. The microcapsule slurry was then left to react for 4 hours to complete modification. The resulting microcapsule slurry was an aqueous slurry of microcapsules which were visible under a light microscope.

### Example of an Added Polymer Component

### Example 5: Modified Amine and Added Zein Protein

Preparation of Microcapsules having polymer shell comprising of 1 mol·eq dipentaerythritol penta/hexaacrylate and 1 mol·eq [2-(methacryloyloxy) ethyl]trimethyl ammonium chloride modified pentaethylene hexamine with zein protein in the polymeric shell, and encapsulation of home care fragrance Sunburst fresh R14-3913. Also with 9:1 weight eq. cationic guar (CG) and xanthan gum (XG) coacervate. (Ref 221-21-1)

A 1:1 mol·eq. modified amine was prepared by heating 10.56 g Pentaethylene hexamine and dissolving 0.22 g (2.1% owo PEHA) of Zein protein in the monomer at 35° C. This monomer solution was then heated to 60° C. 12.59 g (75%) [2-(methacryloyloxy)ethyl]trimethylammonium chloride was added under mechanical stirring. The modified amine was then left to react for 3 hours to complete modification.

An aqueous phase was prepared by mixing 26.02 g of 10% aqueous solution of polyvinyl alcohol and 163.24 g of deionized water. 0.16 g of defoamer was also added.

An acrylate oil phase was prepared by dissolving 5.64 g dipentaerythritol penta/hexaacrylate in 30 g Fragrance Sunburst fresh.

An amine oil phase was prepared by dissolving 4.50 g (85.6%) [2-(methacryloyloxy) ethyl]trimethyl ammonium chloride modified pentaethylene hexamine in 12.35 g of Fragrance Sunburst fresh. 8.47 g of propylene glycol dicaprylate/caprate was added followed by 0.30 g of triethylamine. The amine oil phase was added to the acrylate oil phase under mechanical stirring to form the internal phase. The internal phase was added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 35° C. The oil-in-water emulsion was then left to react for 24 hours to complete polymerization. The pH of the final emulsion was adjusted to 4.5. The slurry was cooled to <10° C. and 2.5 ml's of Glutaraldehyde solution was added. The cooling was then removed, and slurry was left under stirring for at least 6 hrs. The resulting microcapsule slurry was an aqueous slurry of microcapsules which were visible under a light microscope. (221-21-1)

### Examples of Other Coated/Multilayered Capsules

### Example 6

Preparation of Microcapsules having polymer shell comprising of 1 mol. eq Dipentaerythritol penta/hexaacrylate (DiPEHA) and 1 mol. eq Pentaethylene hexamine (PEHA) and encapsulation of home care fragrance Sunburst fresh R14-3913: With secondary shell comprising of 9:1 weight eq. Cationic guar and xanthan gum (XG) coacervate. (Ref 221-3-1)

An aqueous phase was prepared by mixing 10 g of 10% aqueous solution of Polyvinyl alcohol, 81.17 g of cationic guar solution (NHANCE CG) and 24.87 g of deionized water. 0.06 g of defoamer was also added. An acrylate oil phase was prepared by dissolving 7.21 g dipentaerythritol penta/hexaacrylate in 30 g Fragrance Sunburst fresh.

An amine oil phase was prepared by dissolving 2.28 g of pentaethylene hexamine (PEHA) in 12.35 g of Fragrance Sunburst fresh. 8.47 g of propylene glycol dicaprylate/caprate was added followed by 0.28 g of triethylamine. The amine oil phase was added to the acrylate oil phase under mechanical stirring to form the internal phase. The internal phase was added to the aqueous phase under mechanical stirring to form a coarse emulsion. The coarse emulsion was homogenized using an overhead stirrer at 400 rpm for 10 mins. The formed emulsion was transferred to a reactor pot and the emulsion was heated to 35° C. The oil-in-water emulsion was then left to react for 24 hours to complete polymerization. 73.31 g of 0.24% Xanthan gum solution was added to the emulsion and allowed to mix for 30 mins. The pH of the final emulsion was adjusted to 4.5. The slurry was cooled to <10° C. and 2.5 mls of Glutaraldehyde solution was added. The cooling was then removed, and slurry was left under stirring for at least 6 hrs. The resulting microcapsule slurry (221-3-1) was an aqueous slurry of microcapsules which were visible under a light microscope.

### Example 7

Preparation of Microcapsules Having Polymer Shell Comprising of 1 Mol·Eq. Dipentaerythritol Penta/Hexaacrylate (Di-PETA) and 1 Mol·Eq. [2-(Methacryloyloxy) Ethyl]Trimethylammonium Chloride (MBJ; 2—Also Known as Trimethylammonioethyl Methacrylate Chloride) Modified Pentaethylene Hexamine and Encapsulation of Home Care Fragrance Sunburst Fresh R14-3913: With Secondary Shell Comprised of 9:1 Weight Eq. Cationic Guar and Xanthan Gum (XG) Coacervate. (Ref 221-10-1)

A 1:1 mol·eq. modified amine was prepared by heating 10.56 g pentaethylene hexamine to 60° C. 12.59 g (75%) [2-(methacryloyloxy)ethyl]trimethylammonium chloride was added under mechanical stirring. The modified amine was then left to react for 3 hours to complete modification. An aqueous phase was prepared by mixing 10 g of 10% aqueous solution of Polyvinyl alcohol, 81.17 g of cationic guar solution and 24.87 g of deionized water. 0.06 g of defoamer was also added. An acrylate oil phase was prepared by dissolving 5.64 g dipentaerythritol penta/hexaacrylate in 30 g Fragrance Sunburst fresh. An amine oil phase was prepared by dissolving 4.46 g (86.4%) [2-(methacryloyloxy)ethyl]trimethylammonium chloride modified pentaethylene hexamine in 12.35 g of Fragrance Sunburst fresh. 8.47 g of propylene glycol dicaprylate/caprate was added followed by 0.30 g of triethylamine. The amine oil phase was added to the acrylate oil phase under mechanical stirring to form the internal phase. The internal phase was added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 35° C. The oil-in-water emulsion was then left to react for 24 hours to complete polymerization. 73.31 g of 0.24% Xanthan gum solution was added to the emulsion and allowed to mix for 30 mins.

The pH of the final emulsion was adjusted to 4.5. The slurry was cooled to <10° C. and 2.5 mls of glutaraldehyde solution was added. The cooling was then removed, and slurry was left under stirring for at least 6 hrs. The resulting microcapsule slurry (221-10-1) was an aqueous slurry of microcapsules which were visible under a light microscope.

A similar process was followed to make microcapsules with the same fragrance. Capsules were first prepared via the acrylate amine Michael addition, with the inclusion of xanthan gum (XG; post-polymerization addition. i.e. added at the end of the initial capsule shell formation) some examples with an increased loading of N—HANCE CCG 45 to form a coacervate with XG, prepared in a pH=4.5 aqueous solution before mixing into aqueous phase (water+Poval (reduced)). Post homogenization addition of xanthan gum solution, pH 10.5.

The cationic guar and xanthan gum (carboxyl acid functional polysaccharide) form a coacervate at pH 4.5 which is then crosslinked with glutaraldehyde or glyoxal or other crosslinkers. Other combinations of cationic or pH responsive polymers and anionic or pH responsive polymers can form coacervates at similar or different pH conditions. Such conditions can be determined experimentally for the specific pairings chosen. For example, poly quaternary ammonium, or more generally cationic polymers or polymers which can form quaternary ammonium groups or cationic groups, include proteins or peptides, and poly-ß-amino esters as examples. Anionic polymers, or polymers which can form anionic groups, include carboxylic acid or sulfonic/sulfate functional polymers, including functional polysaccharides or functional poly-ß-amino esters.

Examples and variations included:

221-11-1 capsules, prepared from MBS pre modified PEHA with di-PETA and with secondary coating, crosslinked with glutaraldehyde; pH=10.5 adjusted to 4.0 then to 8.6.

221-13-1 capsules, prepared from MBS pre modified PEHA with di-PETA with a higher CCG loading for the secondary coating in this case crosslinked with glyoxal; pH=10.5 adjusted to 4.1 then 8.5.

221-20-1 capsules, prepared from MBJ pre-modified PEHA with di-PETA and secondary coating with a higher CCG loading and pH=10.5, adjusted to 4.5.

221-21-1 capsules prepared from MBJ pre-modified PEHA with secondary coating with added zein and higher CCG and pH=8.8.

Some details further illustrating these examples are described below:

### Example 8

Using a pre-modified amine and added zein protein with a secondary coating: Preparation of Microcapsules having polymer shell comprising of 1 mol·eq. Dipentaerythritol penta/hexaacrylate and 1 mol·eq. [2-(Methacryloyloxy)ethyl]trimethylammonium chloride modified Pentaethylene hexamine with zein protein in the polymeric shell, and encapsulation of home care fragrance Sunburst fresh R14-3913. With secondary shell comprised of 9:1 weight eq. cationic guar and xanthan gum coacervate. Ref 221-21-1.

A 1:1 mol. eq modified amine was prepared by heating 10.56 g Pentaethylene hexamine (PEHA) and dissolving 0.22 g (2.1% on wt of PEHA) of zein protein in the PEHA monomer at 35° C. This monomer solution was then heated to 60° C. 12.59 g (75%) [2-(methacryloyloxy)ethyl]trimethylammonium chloride was then added under mechanical stirring. The modified amine was then left to react for 3 hours at 60° C. to complete the PEHA modification reaction. An aqueous phase was prepared by mixing 10 g of 10% aqueous solution of Polyvinyl alcohol, 81.17 g of cationic guar (Nhance CG45) solution and 24.87 g of deionized water. 0.06 g of defoamer was also added. An acrylate oil phase was prepared by dissolving 5.64 g Dipentaerythritol penta/hexaacrylate in 30 g Fragrance Sunburst fresh. An amine oil phase was prepared by dissolving 4.50 g (85.6%) [2-(methacryloyloxy)ethyl]trimethylammonium chloride modified pentaethylene hexamine in 12.35 g of Fragrance Sunburst fresh. 8.47 g of propylene glycol dicaprylate/caprate was added followed by 0.30 g of triethylamine. The amine oil phase was added to the acrylate oil phase under mechanical stirring to form the internal phase. The internal phase was added to the aqueous phase under mechanical stirring to form a coarse emulsion. The emulsion was homogenized using under overhead mechanical stirring at 400 rpm for 10 mins. The formed emulsion was transferred to a reactor pot and the emulsion was heated to 35° C. The oil-in-water emulsion was then left to react for 24 hours to complete polymerization and initial capsule shell formation. 73.31 g of 0.24% xanthan gum solution was added to the emulsion and allowed to mix for 30 mins. The pH of the final emulsion was adjusted to 4.5. The slurry was cooled to <10° C. and 2.5 mls of glutaraldehyde solution was added. The cooling was then removed, and slurry was left under stirring for at least 6 hrs. The resulting microcapsule slurry was an aqueous slurry of microcapsules which were visible under a light microscope.

### Example 9

Using a Pre-Modified Amine and Zein Protein Additive: Preparation of Microcapsules Having Polymer Shell Comprising of 1 Mol. Eq Dipentaerythritol Penta/Hexa Acrylate and 1 Mol. Eq [2-(Methacryloyloxy) Ethyl]Trimethylammonium Chloride Modified Pentaethylene Hexamine with Zein Protein in the Polymeric Shell, and Encapsulation of Home Care Fragrance Sunburst Fresh R14-3913. Ref 221-23-1.

A 1:1 mol. eq. modified amine was prepared by heating 10.56 g pentaethylene hexamine (PEHA) and dissolving 0.22 g (2.1% owo PEHA) of zein protein in the monomer at 35° C. This amine/polymer solution was then heated to 60° C. 12.59 g (75%) [2-(methacryloyloxy)ethyl]trimethylammonium chloride was added with r mechanical stirring. The modified amine-polymer mixture was then left to react for 3 hours to complete the reactive modification. An aqueous phase was prepared by mixing 26.02 g of 10% aqueous solution of polyvinyl alcohol and 163.24 g of deionized water. 0.16 g of defoamer was also added. An acrylate oil phase was prepared by dissolving 5.64 g dipentaerythritol penta/hexaacrylate in 30 g Fragrance Sunburst fresh. An amine oil phase was prepared by dissolving 4.50 g (85.6%) [2-(methacryloyloxy) ethyl]trimethylammonium chloride modified Pentaethylene hexamine containing zein protein in 12.35 g of Fragrance Sunburst fresh. 8.47 g of propylene glycol dicaprylate/caprate was added followed by 0.30 g of triethylamine. The amine oil phase was added to the acrylate oil phase with mechanical stirring to form the internal phase. The internal phase was added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 35° C. The oil-in-water emulsion was then left to react for 24 hours at this temperature to complete polymerization. The pH of the final emulsion was adjusted to 4.5. The slurry was cooled to <10° C. and 2.5 mls of glutaraldehyde solution was added. The cooling was then removed, and slurry was left under stirring for at least 6 hrs. The resulting microcapsule slurry was an aqueous slurry of microcapsules which were visible under a light microscope.

The modification of the poly-ß-amino ester can be via other reactions or modifications or overcoating procedures at one or more various stages of the process. For example, the modification can be by free radical reactions (including ‘controlled’ radical reactions) of residual or available acrylate groups on the poly ß-amino-ester (PBAE) or its copolymer, or via H abstraction reactions or chain transfer reactions of other parts of the PBAE or its copolymer.

Such co-reactions can be with other radical polymerizable molecules groups such as groups containing double bonds (vinyl groups including conjugated vinyl groups and non-conjugated vinyl groups) whether in (meth)acrylate, itaconate, maleate or as other carbon-carbon (vinylic) double bonds, or also proceeding via hydrogen abstraction or chain transfer or other reactions such as may occur in polysaccharides for example, in the presence of radicals.

For example, radical co-reactions of PBAE, or a PBAE copolymer participating in a radical reaction, can be applied to link them to saccharides as the modifying moiety per se, or to saccharides (mono-, oligo- or poly-saccharides), or to saccharide derivatives (sugar alcohols as example) or to other molecules which are charged or have bulky or hydrophobic groups, and wherein such groups become part of the PBAE based shell wall composition after such reactions or linking.

For example, a saccharide or polysaccharide or a hydrophobically modified saccharide, such as OSA (octenyl succinate modified) starch, may be co-reacted with a poly-ß amino-ester or poly-ß-amino-ester copolymer (e.g. poly-ß-amino-co-ß-thio-ester) via radical reactions.

Such radical reactions can be a pre-reaction and/or occur in the process of shell formation-crosslinking, and/or can be a post-shell formation reaction. In such cases, in some embodiments it may be a poly-ß-amino-ester which is made with excess acrylate groups/functionality to facilitate radical linking to the saccharide, which may or may not have carbon-carbon (vinylic) double bonds on it. Examples illustrating such concepts are described below in Examples 10 and 11.

### Examples of Poly-ß-Amino Ester Capsules Modified with Saccharides

### Example 10: Preparation of Poly-ß-Amino Ester (PBAE) Microcapsules with a Hydrophobic Polysaccharide

This example illustrates the preparation of microcapsules having a polymer shell comprising a PBAE (made via reaction of 2 mol·eq Pentaerythritol tetraacrylate (PETA) and 1 mol·eq 4,4′-Trimethylenedipiperidine (TMPP), so having residual or excess acrylate functionality), hydrophobically modified with 0.002 mol·eq Octenyl succinic anhydride modified waxy corn starch, radically reacted with the PBAE (to introduce hydrophobic groups from the saccharide), for the encapsulation of home care fragrance Sunburst fresh R14-3913. Capsules Ref: (230-11-1)

An oil phase containing a poly-ß-amino ester with excess acrylate functionality was prepared by dissolving 1.41 g of Pentaerythritol tetraacrylate (PETA) and 0.42 g of 4,4′-Trimethylenedipiperidine (TMPP) in 25.50 g of Fragrance Sunburst fresh and 5.10 g of Propylene glycol dicaprylate/caprate under mechanical stirring. The oil phase was heated to 30° C. then left to react for a further 24 hours to form an oligomeric PBAE (with acrylate functionality). 0.13 g of 2,2′-Azodi(2-methylbutyronitrile) was then added as an oil phase radical initiator.

An aqueous phase was prepared by mixing 4.28 g of octenyl succinic anhydride modified waxy corn starch to 90.83 g of deionized water under mechanical stirring. Once fully homogeneous the aqueous phase was heated to 80° C.

An aqueous initiator solution was prepared by dissolving 0.09 g of Sodium persulphate in 10 g of deionized water. The initiator solution was added to the aqueous phase and left to react for 5 minutes. The aqueous phase was cooled to 40° C. 12.24 g of 10% aqueous solution of Polyvinyl alcohol was added.

The oil phase was then added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 40° C. The oil-in-water emulsion was then left to react for 1 hour. The temperature was then increased to 60° C. and left to react for 1 hour. The temperature was then further increased to 80° C. and left to react for 2 hours.

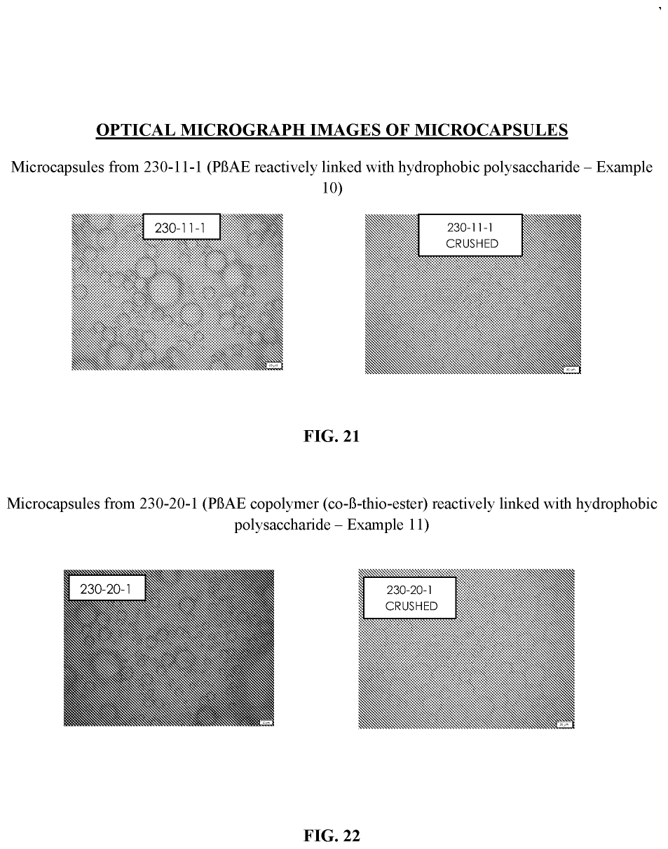

The resulting microcapsule slurry was an aqueous slurry of microcapsules which were visible under a light microscope. Fragrance release from within the microcapsules was strongly evident upon crushing (applying pressure) to the microscope slide (see FIG. 21, Optical Micrograph Images of Microcapsules; capsule reference: 230-11-1).

### Example 11: Preparation of Poly-ß-Amino Ester (PBAE) Copolymer (Poly-ß-Amino Ester-Co-ß-Thio-Ester) Capsules with Hydrophobic Polysaccharide

Microcapsules having a polymer shell comprising a PBAE copolymer, containing ß-amino-ester and ß-thio-ester groups, were made via reaction of 4.3 mol·eq pentaerythritol tetraacrylate (PETA), 1 mol·eq pentaerythritol tetrakis (3-mercaptopropionate) (PTKMP), 0.2 mol·eq 4,4′-trimethylenedipiperidine (TMPP) and 0.005 mol·eq octenyl succinic anhydride modified waxy corn starch (OSA waxy starch) radically reacted with the PBAE copolymer (to introduce hydrophobic groups from the saccharide), for the encapsulation and encapsulation of home care fragrance Sunburst fresh R14-3913. Capsule Ref: 230-20-1.

An oil phase was prepared by dissolving 1.35 g of pentaerythritol tetraacrylate (PETA), 0.44 g of pentaerythritol tetrakis (3-mercaptopropionate)(PTKMP) and 0.04 g of 4,4′-trimethylenedipiperidine (TMPP) in 25.50 g of Fragrance Sunburst fresh and 5.10 g of propylene glycol dicaprylate/caprate under mechanical stirring. The oil phase was heated to 30° C. then left to react for 24 hours. 0.13 g of 2,2′-Azodi(2-methylbutyronitrile) was added.

An aqueous phase was prepared by mixing 4.28 g of octenyl succinic anhydride modified waxy corn starch to 90.87 g of deionized water under mechanical stirring. Once fully homogeneous the aqueous phase was heated to 80° C.

An aqueous phase initiator solution was prepared by dissolving 0.09 g of sodium persulphate in 10 g of deionized water. The initiator solution was added to the aqueous phase and left to react for 5 minutes. The aqueous phase was cooled to 40° C. 12.24 g of 10% aqueous solution of Polyvinyl alcohol was added. The oil phase was added to the aqueous phase under mechanical stirring to form a coarse emulsion. The coarse emulsion was homogenized using an IKA magic lab homogenizer, 1 pass at 4000 rpm.

The formed emulsion was transferred to a reactor pot and the emulsion was heated to 40° C. The oil-in-water emulsion was then left to react for 1 hour. The temperature was then increased to 60° C. and left to react for a further 1 hour. The temperature was then increased to 80° C. and left to react for a further 2 hours.

The resulting microcapsule slurry was an aqueous slurry of microcapsules which were visible under a light microscope. Fragrance release from within the microcapsules was strongly evident upon crushing (applying pressure) to the microscope slide (see FIG. 22, Optical Micrograph Images of Microcapsules; capsule reference: 230-20-1).

The modification of the poly-ß-amino ester or its copolymer(s) can also be via application of an overcoating (which may or may not involve reactions) and/or additional crosslinking. Examples illustrating such concepts are described below in Examples 12 and 13.

### Example 12: Spray Dried Overcoating of a Poly-ß-Amino Ester-Co-ß-Thio-Ester Microcapsule with a Hydrophobic Modifier (Octentyl Succinic Modified Starch—OSA Starch)

This example illustrates the use of a copolymer and the overcoating concept to apply the modifying group (in this case a hydrophobic modifier). (Capsules ref: 229-37-1).

Microcapsules comprising of 1 mol eq pentaerythritol tetraacrylate, 0.1 mol eq 4,4 Trimethylene dipiperidine (TMPP), 0.9 mol eq Pentaerythritol hexakis (3-mercaptopropionate), and home care fragrance sunburst fresh R14-3913 were made according to the procedure described immediately below (and then overcoated).

An oil phase was prepared by dissolving 4.88 g of Pentaerythritol tetraacrylate and 0.58 g of 4,4′-Trimethylenedipiperidine in 25.50 g of Fragrance Sunburst fresh and 5.10 g of Propylene glycol dicaprylate/caprate under mechanical stirring. The oil phase was left to mix for 2 hours at room temperature. In a separate beaker a second oil phase was prepared from 6.50 g of Pentaerythritol hexakis (3-mercaptopropionate) dissolved into 27.92 g of Fragrance Sunburst fresh and 0.36 g of triethylamine, mixed for 5 mins.

An aqueous phase was prepared by mixing 205.9 g of deionized water, 32.81 g of a 10% aqueous solution of polyvinyl alcohol and 0.2 g Agitan 295 defoamer. The aqueous phase was stirred for 5 mins.

The two oil phases were mixed and stirred for 30 seconds. The combined oil phase was added to the aqueous phase and stirred for 1 minute. The coarse emulsion was homogenized with an IKA magic lab at 4000 rpm for 1 pass. The final emulsion was transferred to the reactor flask and reacted at 35° C. for 24 hours to make the initial poly-ß-amino-co-ß-thio-ester capsules.

In readiness for overcoating the capsules, 7.44 g of OSA-modified starch was added to 148.8 g of de-ionized water and mixed at 50° C. for 1 hr and cooled to room temperature. The solution was stirred overnight.

50 g of the pre-prepared microcapsules (made as above—comprising of 1 mol eq pentaerythritol tetraacrylate, 0.1 mol eq 4,4′trimethylene dipiperidine and 0.9 mol eq pentaerythritol hexakis (3-mercaptopropionate) shell and home care fragrance sunburst fresh R14-3913 in the core) was added to the pre-made OSA-modified starch solution. The mixture was homogenized using an IKA Ultraturrax™ mixer, for 1 min at 3000 rpm. 1.25 g of Sipernat 50S (hydrophobically modified silica) was then added to the slurry and left to mix for 30 mins.

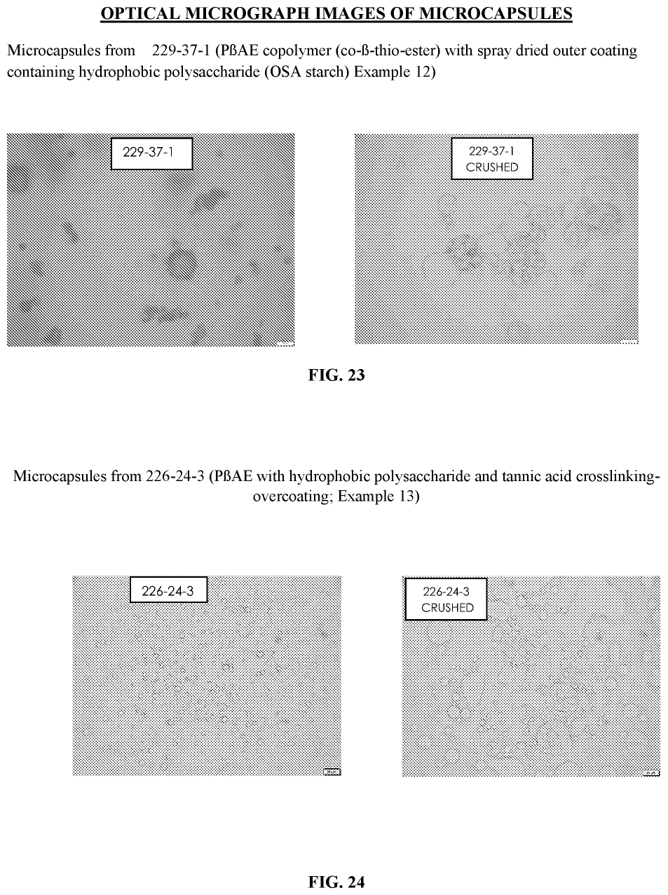

This slurry was spray dried using a Lab Plant SD-06 Spray dryer at 180° C. Overcoated capsules comprising a fragrance inner core inside a poly-ß-amino-ester-co-ß-thio-ester shell overcoated with a hydrophobically modified starch were thus prepared. The spray dried material was collected and observed under the light microscope (see FIG. 23, Optical Micrograph Images of Microcapsules; capsule reference: 229-37-1.). Upon crushing either as dry capsules, or after redispersion into water, a strong burst of fragrance release was evident. Example 13: Poly-8-Amino Ester (PBAE) Capsules Made with a Hydrophobic Saccharide and Additional Coating and/or Crosslinking with Tannic Acid (Preparation of Microcapsules Having Polymer Shell Comprising of 2 Mol·Eq Pentaerythritol Tetraacrylate, 1 Mol·Eq 4,4′-Trimethylenedipiperidine, 0.002 Mol·Eq Octenyl Succinic Anhydride Modified Waxy Corn Starch and 0.06 Mol·Eq Tannic Acid and Encapsulation of Home Care Fragrance Sunburst Fresh R14-3913. Capsule Ref: 226-24-3.)

An oil phase was prepared by dissolving 1.41 g of pentaerythritol tetraacrylate (PETA) and 0.42 g of 4,4′-trimethylenedipiperidine (TMPP) in 25.50 g of 2-Propanol and 5.10 g of propylene glycol dicaprylate/caprate under mechanical stirring. The oil phase was heated to 80° C. and left to react for 2 hours. The oil phase was cooled to 30° C. and the solvent or majority of solvent, was removed by evaporation from an open reactor under fume hood extraction for 24 hours.

An oil phase initiator was prepared by dissolving 0.13 g of 2,2′-Azodi(2-methylbutyronitrile) in 25.50 g of Fragrance Sunburst fresh. The oil phase initiator was added to the oil phase.

An aqueous phase was prepared by mixing 4.28 g of octenyl succinic anhydride modified waxy corn starch to 90.83 g of deionized water under mechanical stirring. Once fully homogeneous the aqueous phase was heated to 80° C.

An aqueous phase initiator solution was prepared by dissolving 0.09 g of Sodium persulphate in 10 g of deionized water. This initiator solution was added to the aqueous phase and left to react for 5 minutes. The aqueous phase was cooled to 40° C. 12.24 g of 10% aqueous solution of polyvinyl alcohol was added.

The oil phase was then added to the aqueous phase under mechanical stirring to form a coarse emulsion. The coarse emulsion was homogenized using an IKA magic lab homogenizer, 1 pass at 4000 rpm. The formed emulsion was transferred to a reactor pot and the emulsion was heated to 40° C. The oil-in-water emulsion was then left to react for 1 hour.

The reaction temperature was then increased to 60° C. and left to react for a further 1 hour. The reaction temperature was then increased to 80° C. and left to react for a further 2 hours. Microcapsules containing fragrance inside were formed (within a slurry).

Additional Post-Encapsulation Process/Layer: A solution was prepared by dissolving 0.21 g of Tannic acid in 10 g of deionized water. The reaction mixture (slurry) above containing the fragrance microcapsules was cooled, or allowed to cool, to 50° C. after the 80° C. reaction stage. The tannic acid solution was added to the microcapsule slurry and left to react for 4 hours. The resulting modified microcapsule slurry was an aqueous slurry of microcapsules which were visible under a light microscope, and which clearly released fragrance upon crushing (see FIG. 24, Optical Micrograph Images of Microcapsules; capsule reference: 226-24-3).

In all cases described in the various examples above and their variants, microcapsules were formed and clearly released fragrance upon crushing under a microscope slide. Images of examples of examples of microcapsules are shown further below. For those, microcapsules prepared with modifying reactants introducing the specific targeted attributes or features (either in pre-reactions, post-reactions or via selected mixtures of monomeric reactants) typically more structured capsule images were observed, and, with all such cases of modification, the fragrance bloom test was significantly improved compared to a basic or classical unmodified amine—acrylate system. This indicates superior performance for the modified systems in the most demanding of formulations or products made by which ever route is applied and would include in-situ and interfacial oil in water polymerizations. For other combinations such as those without the specific targeted modifications or specifically adjusted stoichiometries, they are able to be made via the in-situ oil in water polymerization route and are able to be used in some formulated products—especially where the formulations are considered less demanding or challenging. In modified systems tested after aging (for example at 40° C. for 2 or 4 weeks in formulated product such as fabric conditioner, typically around pH3) fragrance bloom tests after aging of the modified systems were typically well retained and in the main better than bloom test outcomes after aging for unmodified equivalents (unmodified amine-acrylate). These data indicate storage stable microcapsules which are also biodegradable can be made via the approaches of this invention.

In addition, biodegradation tests run on some of the modified system examples indicated satisfactory evidence of biodegradation according to OECD 301F in activated sludge and reached in excess of 20% biodegradation after 28 days or after 60 days, with ongoing biodegradation indicated.

Details of these test methods and example results are given below:

### Biodegradation Testing

### Example 14

This was carried usually out according to OECD methods. For example, methods such as OECD 301D, 301F, 302B, 306, were variously used, some over extended timelines. Samples that are insoluble in aqueous media often require development for a suitable dispersion or form for the test. In some cases, the EN 14852:2018 or EN ISO 14851:2004 test can be used, which runs for time period of 6 months in aquatic media (and is also cited, along with others such as those above, in ECHA draft protocols for avoidance of microplastics concerns). Innocula and suitable water (secondary effluent surface water, seawater or activated sludge) were used as supplied from a local sources such as a wastewater treatment plant. A mineral medium specified by the OECD 301D method and the inoculum were added to deionized water which was subsequently aerated for 20 minutes prior to addition of the sample polymer sample at a concentration of 4-10 mg/ml depending on predicted biodegradability.

In some examples, biodegradation was monitored from measurements of dissolved oxygen content. In some examples this test is done in fresh water using inoculum supplied by a local water treatment plant. This test is used to mimic the environment these polymers will be in after going through a freshwater waste treatment plant. This test uses a readily biodegradable sodium benzoate reference as a positive control. All samples are run in duplicate. Measurements were taken approximately at 7 day intervals to at least 28 days and in many cases beyond. Example data is given in the table below.

Biodegradability measurements of Example capsule shells (301F Activated Sludge)

There is a balance for some applications where a low, but perceptibly ongoing, biodegradation rate can be accepted, and where combined with for example a higher fragrance or other lipophilic cargo compatibility and/or higher water resistance, can still make a desirable end product which will be non-persistent.

### Examples Samples for Biodegradation Testing (Michael Addition Polymerizations)

Equivalent compositions (analogous shell materials) of the microcapsules described above could also be made, without fragrance, using dichloromethane as another lipophilic cargo, which was, for the purposes of testing, then subsequently evaporated to leave polymeric shell material only, for use in biodegradation testing.

Example encapsulated DCM solvent (subsequently removed by evaporation) for biodegradation testing of polymeric shell material; Same procedure for the capsule formation was used but DCM was used in place of fragrance. Following completion of the shell formation (formed around DCM) the mixture was transferred to a beaker with a magnetic stirred bar and allowed to stir in a fume hood for 72 hours to allow evaporation of the dichloromethane. No solvent was detected via GC following this. This dispersion was assessed for biodegradability via 301F, using an activated sludge inoculum.

Representative samples prepared with dichloromethane cargo subsequently evaporated is similar to (equivalent shell prepared with fragrance, described above, which showed a fragrance release/bloom after formulation into a representative fabric conditioner system), showed biodegradation after 28, 30, 40 days or 60 days in the OECD 301F test using activated sludge sourced from a local water treatment plant (Yorkshire Water) and showed ongoing biodegradation thereafter.

220-83-1 was a microcapsule shell made (with DCM as cargo removed) from LMA pre-modified (4 hr 60° C. pre-reaction) at 1:1 mol ratio (LMA:PEHA) subsequently then reacted in an encapsulation process reaction with di-PETA. This sample showed biodegradation of 15% after 26 days and 18% after 40 days in the OECD 301F test with activated sludge and with ongoing biodegradation thereafter. This capsule shell material is equivalent to that used in 220-61-1 which exhibits excellent fragrance bloom in fabric conditioner (see below) and good retention of a bloom performance after 40° C. aging (see below).

220-84-1 was a microcapsule shell made (with DCM as cargo removed) from MBJ pre-modified (4 hr/60° C. pre-reaction) at 1:1 mol ratio (MBJ:PEHA)), subsequently then reacted in an encapsulation process reaction with di-PETA. This sample showed biodegradation of 35% after 26 days, and 46% after 40 days, in the OECD 301F test with activated sludge and with ongoing biodegradation thereafter. This capsule shell material is equivalent to that used in 220-61-2 which exhibits excellent fragrance bloom in fabric conditioner (see below) and good retention of a bloom performance after 40° C. aging (see below).

These data indicate storage stable microcapsules which are also biodegradable can be made via the approaches of this invention.

### Sensory and Fragrance Release Testing

### Example 15

Examples of microcapsules with fragrance inside prepared by in-situ oil in water Michael Addition polymerization displaying a fragrance bloom (triggered release (rubbing) via sensory panel testing) after formulating into a fabric conditioner base are summarized below.

### Test Procedures for Samples for Fragrance Bloom Testing (Fabric Conditioner Base):

Capsule slurries were tested in blind sensory evaluations (fragrance bloom tests) with a collection of people (minimum 2, typically 3-5). Note: Microcapsules produced typically contained ˜15-30 wt % fragrance encapsulated in the slurry—most used in the data reported were ˜17 wt %.

### Typical Pre-Screening Sensory Test

A test mixture of the slurry is prepared using 18 g of a fabric conditioner/softener formulation and an amount of slurry such that the fragrance loading in the test mixture is 0.1 g fragrance (based on the fragrance amount encapsulated in a slurry) and water added to make 20 g of test mixture.

In parallel a fabric wash mixture was prepared using each test mixture, each in a 2-litre beaker using an overhead stirrer at 250 rpm. This comprised 2 g of each test mixture above and 998 g of water (tap). Small squares (approx. 75 mm×75 mm; number is according to the number of people testing the samples of that slurry) of towel material were added to the beaker and stirred for 5 minutes after which they were removed and hung to dry in the air overnight, for 16 hours.

The next day another person or people (who did not make up the samples) smelt (sniffed) an untreated towel and then sniffed a sample of pure fragrance and marked each in terms of an intensity number for these reference points, between 1 to 9 (9=highest fragrance intensity—as in neat fragrance typically). Randomly, samples prepared on towels as described were selected and sniffed and an intensity number recorded. Then the towel material is rubbed together for 5 seconds and sniffed again. The intensity after rubbing (post rub) was also recorded. This was repeated at random until all samples are tested as such by each panel member.

An average intensity is calculated for a before (pre-rub) and after (post rub) the rubbing for each sample. Example data are show further below.

Detailed Sensory Test: In another test method, a terg-o-tometer was used.

### Example 16: Fabric/Conditioner Base Formulation

A prototype fabric softener/conditioner base formulation for such testing comprised of:

| | Base fabric |
|---|---|
| Ingredient | conditioner (wt. %) |
| Phase A | |
| Deionized water | 83.85 |
| Methyl bis[ethyl(tallowate)]-2-hydroxyethyl | 5.55 |
| ammonium methyl sulfate - Stepantex VT-90, | |
| Stepan Co. | |
| Poly(2-dimethylamino) ethyl methacrylate methyl | 0.5 |
| chloride quaternary salt | |
| Chloromethyl isothiazolinone (CMIT) and | 0.1 |
| methylisothiazolinone (MIT) - Troy Guard CM1.5, | |
| Troy corp. | |
| Total | 90.00 |

Prototype fabric softener/conditioner with a hole of 10% to accommodate for other ingredients to be added later. Prepare phase B containing 0.5% active neat fragrance or fragrance encapsulate by pre-mixing with an equal active amount of emulsifier such as Tomadol 1-73B to be added to the fabric conditioner base phase A. The conditioner is then balanced with deionized water. Finally, pH of the prepared fabric conditioner base is adjusted to pH 2.5-3.5 with weak acid, if needed. The Brookfield viscosity of the prepared base varies between 100-600 cP, depending on fragrance encapsulate test material. All capsules tested were observed to be stable in the formulation.

Examples of data from such tests (pre-screening fragrance bloom in fabric conditioner formulation ˜pH 3)) are summarized below. This data will indicate potential for use as triggered release capsules.

Where there is significant difference between a pre-rub and post-rub assessment (A) such potential is indicated. For example, a A of 3 or more is a good indicator of some such potential in some applications though a higher A is better. In parallel though stability aging (see later) is indicative of longevity of performance and storage stability. For some applications this is more readily achieved though for laundry applications such as fabric conditioners, this is often more challenging due to the relative aggressiveness of such formulations.

### Example 17

Fragrance Bloom Data from Sensory Tests: Pre-Screening Fragrance Bloom Tests in Fabric Conditioner Formulation—Pre- and Post-Rubbing of Treated Fabrics. Higher More Intensity of Fragrance:

TABLE 1
Screening Fragrance Bloom Sensory Tests

| Sample No. | Pre-rub | Post-rub | Sample Description |
|---|---|---|---|
| 220-02-1 | 1 | 5 | PEHA-Di-PETA (equimolar on reactive groups) |
| 220-26-2 | 1.5 | 4.5 | HMDA-Di-PETA (equimolar on reactive groups) |
| 220-40-2 | 2.5 | 6.5 | BA (2 mol eq) pre-modified PEHA with di-PETA |
| 220-42-2 | 1.5 | 5 | SA (2 mol eq) pre-modified PEHA with di-PETA |
| 220-43-2 | 1 | 4 | BA (2 mol eq) pre-modified PEHA with di-PETA |
| 220-44-1 | 1 | 4.5 | IBA pre-modified PEHA with di-PETA |
| 220-45-1 | 3.5 | 8 | MBS pre-modified PEHA with di-PETA |
| 220-45-2 | 1 | 6 | MBS (2 mol eq) pre-modified PEHA with di-PETA |
| 220-46-1 | 2 | 7 | APTAC pre-modified PEHA with di-PETA |
| 220-55-1 | 2 | 6.5 | LC (lauroyl chloride) pre-modified PEHA with di- |
| | | | PETA |
| 220-57-1 | 2 | 6 | t-BA modified PEHA with di-PETA |
| 220-58-1 | 2.5 | 7 | t-BMA modified PEHA with di-PETA |
| 220-60-1 | 1.5 | 6.5 | SMA pre-modified PEHA with di-PETA |
| 220-61-1 | 1.5 | 7.5 | LMA pre-modified PEHA with di-PETA |
| 220-62-1 | 1.5 | 8 | MBJ pre-modified PEHA with di-PETA |
| 220-63-1 | 2.5 | 8 | MAPTAC BA pre-modified PEHA with di-PETA |
| 220-70-1 | 2 | 7 | PEHA-di-PETA (1:1 overall stoichiometry on |
| | | | reactive groups |
| 220-71-1 | 2.5 | 7 | PEHA-di-PETA (1:1 overall stoichiometry on |
| | | | reactive groups (repeat) |
| 220-73-1 | 5 | 9 | LMA post-modified PEHA with di-PETA |
| 220-73-2 | 2.5 | 8 | t-BMA post-modified PEHA with di-PETA |
| 220-73-3 | 3 | 8 | MBJ post-modified PEHA with di-PETA |
| 220-73-4 | 3.5 | 8 | MAPTAC post-modified PEHA with di-PETA |
| 220-79-1 | 2.5 | 8 | LMA pre- modified PEHA (2:1) with di-PETA |
| 220-80-1 | 3.5 | 9 | MBJ (2:1) pre-modified PEHA with di-PETA |
| 220-81-1 | 3 | 8 | MBJ (1)-LMA (1) pre-modified PEHA with di- |
| | | | PETA (MBJ pre-modification first) |
| 220-82-1 | 3.5 | 8.5 | LMA (1)- MBJ (1) PEHA pre modified PEHA with |
| | | | di-PETA (LMA pre modification first) |
| 220-88-1 | 1.5 | 8.5 | MBJ (1) pre-modified PEHA (1) with PETA tetra- |
| | | | acrylate |
| 220-89-1 | 2 | 7 | MBJ (1) pre-modified PEHA (1) with PETA tri- |
| | | | acrylate |

### Example 18

TABLE 2
Pre-Screen Fragrance Bloom Sensory Tests on secondary Coated Samples

| Sample No | Pre-rub | Post-rub | Sample description |
|---|---|---|---|
| 221-08-1 | 2.5 | 7.5 | With secondary coating |
| 221-09-1 | 2.5 | 8 | With secondary coating |
| 221-11-1 | 2 | 8.5 | MBS pre modified PEHA with di-PETA and with |
| | | | secondary coating all crosslinked with |
| | | | glutaraldehyde; pH = 10.5 adjusted to 4.0 then to 8.6 |
| 221-13-1 | 2 | 8.5 | MBS pre modified PEHA with di-PETA with higher |
| | | | CCG and with secondary coating: crosslinked with |
| | | | glyoxal; pH = 10.5 adjusted to 4.1 then 8.5 |
| 221-20-1 | 4 | 7.5 | MBJ pre-modified PEHA with di-PETA and |
| | | | secondary coating with higher CCG and pH = 10.5 |
| | | | adjusted to 4.5 |
| 221-21-1 | 2.5 | 8.5 | MBJ pre-modified PEHA with secondary coating |
| | | | with added zein, higher CCG and pH = 8.8 |

For some samples another method was followed:

Rinse time: 5-min. at 100 rpm agitation. After rinse, squeeze excess water; Dry: Airline dry overnight. Terg-o-tometer test methodology:

Water hardness: 200 ppm (3Ca2+/1Mg2+); Temperature: 100° F.; Fabric conditioner: 2 g/L. Test fabrics: Pre-conditioned cotton terry towel 12×12 inches cut into 3×3 inches squares, use 10 pieces per 1 L wash soln. Rinse time: 5-min. at 100 rpm agitation. After rinse, squeeze excess water; Dry: Airline dry overnight.

Sensory panel evaluation: Expert panelist are asked to smell references before scoring samples for fragrance intensity score=1 least intense and score=9/10 most intense. Afterwards, panelist is given one treated towel sample blindly to score for relative fragrance intensity before and after rubbing.

Sensory panel evaluation: Expert panelist are asked to smell references before scoring samples for fragrance intensity score=1 least intense and score=9/10 most intense. Afterwards, panelist is given one treated towel sample blindly to score for relative fragrance intensity before and after rubbing.

### Stability Testing

Samples of formulated products from the pre-screen bloom test, formulated as described above for that test, were placed in an oven at 40° C. and periodically (2 weeks or 3 weeks or 4 weeks) removed to perform another pre-screen bloom test, performed as described above. Comparisons of this data will show which materials or polymeric shells will have better storage stability in such an aggressive formulation as a fabric conditioner, known to be among the most demanding of applications for triggered release capsules and the example capsules show good retention of bloom performance after aging in a fabric conditioner formulation which is around pH3.

Data are tabulated below.

### Example 19

Fragrance Bloom Data from Sensory Test after Aging: Pre-Screening Fragrance Bloom Tests in Fabric Conditioner Formulation—Pre- and Post-Rubbing of Treated Fabrics after 2 Weeks of Aging. Higher=More Intensity of Fragrance.

TABLE 3
Pre-screening fragrance bloom tests in fabric conditioner
formulation - pre- and post- rubbing of treated fabrics

| Sample | Pre- | Post- | Aged | Aged | |
|---|---|---|---|---|---|
| No | rub | rub | Pre-rub | Post-rub | Sample description |
| 220-73-1 | 5 | 9 | 2.5 | 8.5 | LMA post modified PEHA with di-PETA |
| 220-73-2 | 2.5 | 8 | 1.5 | 7.5 | t-BMA post-modified PEHA with di-PETA |
| 220-73-3 | 3 | 8 | 1.5 | 8 | MBJ post-modified PEHA with di-PETA |
| 220-73-4 | 3.5 | 8 | 1 | 6.5 | MAPTAC post-modified PEHA with Di-PETA |
| 220-92-3 | 2.5 | 8.5 | 3 | 8.5 | CEA post modified PEHA with di-PETA |
| 220-92-4 | 1.5 | 8 | 2.5 | 7.5 | AA post modified PEHA with di-PETA |
| 220-05-1 | 2.5 | 8.5 | 1 | 6.5 | AMPS pre-modified PEHA with di-PETA |
| 220-06-1 | 2 | 8 | 1 | 7.5 | CEAO pre-modified PEHA with di-PETA |
| 220-07-1 | 1.5 | 5.5 | 1 | 7.5 | CEA pre-modified PEHA with di-PETA |
| 220-08-1 | 1 | 8.5 | 1 | 8 | AA pre-modified PEHA with di-PETA |
| 220- 45-1 | 1 | 6 | 2.5 | 8 | MBS pre-modified PEHA with di-PETA |
| 220-88-1 | 1.5 | 8.5 | 2.5 | 6.5 | MBJ modified PEHA with PETA tetra-acrylate |
| 220-90-1 | 2 | 7 | 1.5 | 8.5 | PEHA-diPETA with excess amine: 1:1.1 |
| | | | | | acceptor:NH |

### Example 20

Fragrance Bloom Data from Sensory Test after Aging:

Pre-screening fragrance bloom tests in fabric conditioner formulation— pre- and post-rubbing of treated fabrics after 4 weeks of aging. Higher=more intensity of fragrance.

TABLE 4
Fragrance Bloom data from Sensory Test after Aging

| | | | Aged | Aged | |
|---|---|---|---|---|---|
| Sample No | Pre-rub | Post-rub | Pre-rub | Post-rub | Sample description |
| 220-40-2 | 2.5 | 6.5 | 3.5 | 8 | BA (2 mol eq) pre-modified PEHA with di-PETA |
| 220-45-1 | 1 | 6 | 2.5 | 9 | MBJ pre-modified PEHA with di-PETA |
| 220-46-1 | 2 | 7 | 2.5 | 7 | APTAC pre-modified PEHA with di-PETA |
| 220-57-1 | 2 | 6 | 2.5 | 8 | t-BA pre-modified PEHA with di-PETA |
| 220-58-1 | 2.5 | 7 | 2 | 8 | t-BMA pre-modified PEHA with di-PETA |
| 220-61-1 | 1.5 | 7.5 | 5 | 8 | LMA pre-modified PEHA with di-PETA |

These data indicate storage stable microcapsules which are also biodegradable can be made via the approaches of this invention.

While the compositions and methods of the disclosed and/or claimed inventive concept(s) have been described in terms of particular aspects, it will be apparent to those of ordinary skill in the art that variations may be applied to the compositions and/or methods and in the steps or in the sequence of steps of the method described herein without departing from the concept, spirit and scope of the disclosed and/or claimed inventive concept(s). All such similar substitutes and modifications apparent to those skilled in the art are deemed to be within the spirit, scope and concept of the disclosed and/or claimed inventive concept(s).

1. A microcapsule comprising:
(i) a polymeric microcapsule shell; and
(ii) a lipophilic core;
wherein, the polymeric microcapsule shell comprises a poly-ß-amino-ester polymer, a co-polymer of poly-ß-amino-ester, a terpolymer of poly-ß-amino-ester, a crosslinked polymer of poly-ß-amino-ester, or mixtures thereof,
wherein, the microcapsule is storage stable, and its polymeric shell is biodegradable.

2. The microcapsule according to claim 1, wherein the poly-ß-amino-ester based microcapsule shell further comprises or is linked to hydrophobic, sterically hindered, charged or pH responsive functional groups or molecules.

3. The microcapsule according to claim 1, wherein the poly-ß-amino-ester is derived from a Michael or conjugate Addition reaction of at least one amine donor and at least one acceptor, wherein the at least one donor component and at least one acceptor component each have a reactive functionality of at least two.

4. The microcapsule according to claim 1, wherein the poly-ß-amino-ester, derived from a Michael or conjugate Addition reaction of at least one amine donor and at least one acceptor, wherein the at least one donor component and at least one acceptor component each have a reactive functionality of at least three.

5. The microcapsule according to claim 1, wherein the poly-ß-amino-ester is a cross-linked poly-ß-amino-ester.

6. The microcapsule according to claim 3, wherein the amine donor is a difunctional primary amine, a multifunctional primary amine, a difunctional secondary amine, a multifunctional secondary amine, or combinations thereof.

7. The microcapsule according to claim 3, wherein the amine donor has C2-C20 aliphatic chain functionality, a C4-C7 cyclic ring functionality or a C4-C7 heterocyclic ring functionality.

8. The microcapsule according to claim 4, wherein the amine donor is selected from the group consisting of any primary alkylamine or primary cyclo-alkylamine, 4,4′trimethylenepiperidine (TMPP), isophorone diamine (IPD), bis-(aminomethyl) cyclohexane, cyclohexane diamine, piperazine, aminoethylpiperazine, bis-amino-norbornane, ethylene diamine, diethylene triamine, diethylene diamine, triethylene tetramine, tetraethylene pentamine, pentaethylene hexamine (PEHA), tris-(2-aminoethyl)amine, bis-(3-aminopropyl) amine, spermine, hexamethylenediamine (HMDA), diamino-propane, diamino-butane, diamino-pentane, diamino-octane, diamino-decane, diamino dodecane, amino-ethanol amino-propanol, amino-butanol, amino pentanol, polyfunctional amine, and polyethyleneimine or derivatives of such di- or multi-functional amines with available amine groups for reaction with acceptors.

9. The microcapsule according to claim 3, wherein the acceptor is selected from the group consisting of an acrylate, methacrylate, maleate, fumarate, itaconate, malonate, crotonate, citraconate, maleimide and mixtures thereof.

10. The microcapsule according to claim 3, wherein the acceptor is selected from the group consisting of:
(a) an itaconate or maleate containing polyester,
(b) an acrylate, diacrylate, or multifunctional acrylate of a polyester;
(c) an acrylate, diacrylate, or multifunctional acrylate of an epoxide;
(d) an acrylate, diacrylate, or multifunctional acrylate of a urethane;
(e) an acrylate, diacrylate, or multifunctional acrylate of a polyether or a diol or a polyol;
(f) an acrylate, diacrylate, or multifunctional acrylate of an amine;
(g) methacrylate analogue of (b) to (f) components, and combinations thereof.

11. The microcapsule according to claim 10, wherein the acceptor is selected from the group consisting of butanediol diacrylate, trimethylol propane triacrylate, pentaerythritol triacrylate, pentaerythritol tetra-acrylate, dipentaerythritol penta-acrylate, dipentaerythritol hexa-acrylate and methacrylate analogues thereof.

12. The microcapsule according to claim 3, wherein the acceptor or donor component is a monofunctional acceptor or a monofunctional donor.

13. The microcapsule according to claim 12, wherein the mono functional acceptor or monofunctional donor comprises hydrophobic, sterically hindered, charged or pH responsive functional groups.

14. The microcapsule according to claim 1, wherein the polymeric microcapsule shell further comprises added zein, protein, polypeptide, polysaccharide, oligosaccharide, monosaccharide, or a sugar alcohol, or polyol, or polymer having hydrophobic, sterically hindered, charged or pH responsive functional groups.

15. The microcapsule according to claim 2, wherein the hydrophobic or sterically hindered, charged, pH responsive functional groups are selected from methyl, ethyl, propyl, butyl, C₅-C₂₀ alkyl, C₅-C₂₀ branched alkyl, tertiary methyl, tertiary ethyl, tertiary propyl, tertiary butyl, cyclohexyl, alkyl-cyclohexyl, isobornyl, norbornyl, menthyl, cholesteryl, cycloaliphatic, phenyl, phenoxy ethyl, benzyl, and aryl moieties.

16. The microcapsule according to claim 2, wherein the hydrophobic, sterically hindered, charged or pH responsive functional groups are selected from the group comprising carboxylic acid, carboxylate, sulfonic acid, sulfonate, phosphonic acid, phosphate, boronic acid, borate, quaternary ammonium, ammonium, tertiary amine, secondary amine, primary amine, amine containing heterocyclic bases, amino-acids, salts, conjugates, and derivatives thereof.

17. The microcapsule according to claim 14, wherein the added zein, protein, polypeptide, polysaccharide, oligosaccharide, monosaccharide, modified sugar alcohol, or the functional polymer is present as a blended or dispersed additive or is co-reacted with, or reactively linked to, a poly-ß-amino ester component.

18. The microcapsule according to claim 1, wherein the lipophilic core is selected from the group comprising agrochemicals, aliphatic esters, anti-microbial agents, anti-fungal, anti-fouling agents, antioxidants, anti-viral agents, biocides, catalysts, cosmetic actives, colorants, dyes, detergents, edible oils, emollient oils, essential oils, fats, fatty acids, fatty acid esters, food additives, flavors, fragrances, hair care actives, halogenated compounds, hydrocarbons, insecticides, insect repellants, lipids, lipophilic scale inhibitors, mineral oil, oral care actives, organic solvents, organic esters, chlorinated solvents, pesticides, perfumes, preservatives, skin care actives, UV absorbers, vegetable oils and combinations thereof.

19. The microcapsule according to claim 18, wherein the core is a fragrance, a perfume, or an essential oil.

20. The microcapsule according to claim 1, wherein the microcapsule is used in a consumer care composition selected from the group consisting of laundry care composition, oral care composition, hair care composition, skin care composition, cosmetic care composition, home care composition and cleaning composition.

21. The microcapsule according to claim 20, wherein the microcapsule is used in a fabric conditioner composition or a laundry detergent composition.

22. The microcapsule according to claim 1, wherein the polymeric microcapsule shell is biodegradable in an aquatic medium or in solid medium or is compostable.

23. The microcapsule according to claim 22, wherein the aquatic or solid medium is selected from group consisting of activated sludge, secondary effluent, river water, surface water, fresh water, sea water, soil, and compost.

24. The microcapsule according to claim 22, wherein the polymeric microcapsule shell material shows a biodegradation rate of at least 20% in an aquatic medium when measured by an OECD Test method 301, 302 or 306.

25. The microcapsule according to claim 22, wherein the polymeric microcapsule shell material shows evidence of biodegradation within 120 days or within 60 days or within 40 days or within 28 days.

26. The microcapsule according to claim 1, wherein the microcapsule is storage stable as a core-shell capsule in an aqueous slurry, in a water-based formulation or in a solvent-based formulation.

27. The microcapsule according to claim 1, wherein the microcapsule is storage stable as a core-shell capsule in a solid, largely waterless formulation or in a printed product.

28. The microcapsule according to claim 26, wherein the microcapsule shows a retained triggered release of cargo or ‘a bloom’ after storing or aging in respective medium for at least 4 weeks at ambient temperature (15-25° C.), or at least 6 weeks or at least 8 weeks or at least 12 weeks at ambient temperature (15-25° C.).

29. The microcapsule according to claim 26, wherein the microcapsule shows a retained triggered release of cargo or ‘a bloom’ after storing or accelerated aging in respective medium for at least 2 weeks, for at least 3 weeks, for at least 4 weeks, for at least 6 weeks, for least 8 weeks, for at least 10 weeks, or for at least 12 weeks at an elevated temperature of 40° C.

30. The microcapsule according to claim 26, wherein the microcapsule shows a retained triggered release of cargo or ‘a bloom’ after storing or aging in a liquid laundry detergent or conditioner formulation of acidic pH for at least 4 weeks at ambient temperature (15-25° C.), or at 40° C., or at least 6 weeks or at least 8 weeks or at least 12 weeks at ambient temperature (15-25° C.), or at 40° C.

31. The microcapsule according to claim 26, wherein the formulation or aging medium has pH range of 3 to 11, 3 to 6, 6 to 8, or 8 to 12.

32. The microcapsule according to claim 26, wherein the formulation or aging medium has pH in the range of 3 to 4, 4 to 5, 5 to 6, 6 to 7, 7 to 8, 8 to 9, 9 to 10 or 10 to 11, or 11 to 12.

33. The microcapsule according to claim 1, wherein the microcapsule is stable as a core-shell capsule in an aqueous slurry or in a water-based formulation having pH in the range of 3 to 5.

34. The microcapsule according to claim 1, wherein the microcapsule is stable as a core-shell capsule in an aqueous slurry or in a water-based formulation having pH in the range of 9 to 11.

35. The microcapsule according to claim 26, wherein the formulation is selected from the group consisting of laundry detergent, fabric softener, fabric conditioner, shampoo, hair conditioner, liquid soap, solid soap, skin deodorant, skin moisturizer, skin conditioner, hair or skin protectant, cleanser, sanitizer, cleaning fluid, dishwashing fluid, dishwashing tablet, washing powder, washing tablet, washing liquid, and cosmetic formulation.

36. The microcapsule according to claim 1, wherein the microcapsule is a double layered microcapsule, a multi-layered microcapsule or an overcoated microcapsule.

37. The microcapsule according to claim 36, wherein the double layered, multilayered or an overcoated microcapsule comprises within its outer coating
(i) a polysaccharide, a protein, a hydrogel, a coacervate, a polysaccharide, an oligosaccharide, a monosaccharide, a polyphenol, a polymer or other molecule comprising hydrophobic, charged or pH responsive groups or combinations thereof, or
(ii) a formulation of polymers comprising at least one component selected from the group consisting of polysaccharide, protein, hydrogel, coacervate, polysaccharide, oligosaccharide, monosaccharide, or a sugar alcohol or polymer comprising hydrophobic, charged or pH responsive groups or combinations thereof.

38. The microcapsule according to claim 36, wherein the double layered, multilayered or an overcoated microcapsule comprises within its outer coating a xanthan gum, polysaccharide gum, a polysaccharide, an alginate polymer, a cellulose ether including hydroxyethyl cellulose or carboxymethyl cellulose, a guar or modified guar including cationic guar, zein or other protein, a polypeptide, a hydrogel, a coacervate, a sugar alcohol or polymer comprising hydrophobic, sterically hindered, charged or pH responsive groups, or a hydrophobically modified starch such as octenylsuccinate modified starch or dodecenyl succinate modified starch.

39. The microcapsule according to claim 1, wherein the microcapsule has an average diameter of about 100 nm to 150 μm or about 1 μm to 100 μm.

40. A method for preparing a microcapsule of claim 1, the method comprising:
(a) preparing an oil-in-water emulsion of (i) an oil phase comprising at least one multifunctional amine donor with at least one multifunctional acceptor, and at least one lipophilic core; and (ii) a water phase comprising at least one stabilizer, at least one defoamer, or at least one emulsifier,
(b) optionally adding at least one catalyst, at least one diluent or at least one initiator to the oil phase;
(c) heating the oil-in-water emulsion with stirring to a temperature between 25° C. and 100° C. and forming the polymeric microcapsule shell by an in-situ oil-in-water reaction of the amine donor(s) with the acceptor component(s); and
(d) obtaining the lipophilic core encapsulated in a polymeric microcapsule shell.

41. The method according to claim 40, wherein the amine donor and acceptor component is functionalized with hydrophobic, sterically hindered, charged or pH responsive functional groups.

42. The method according to claim 40, wherein the amine donor and acceptor comprises monofunctional component and act as Michael addition reactant and wherein, the monofunctional component is functionalized with hydrophobic, sterically hindered, charged or pH responsive functional groups.

43. The method according to claim 42, wherein the functional groups are incorporated into the polymeric shell.

44. A method for preparing a microcapsule of claim 1, the method comprising:
(a) pre-reacting at least one multifunctional amine donor with a modifying reagent to form a modified amine donor;
(b) preparing an oil-in-water emulsion of (i) an oil phase comprising at least one amine functional donor including at least one modified amine donor as prepared in (a), with at least one acceptor(s), and at least one lipophilic core; and (ii) a water phase comprising at least one stabilizer, at least one defoamer or at least one emulsifier,
(c) optionally adding at least one catalyst, at least one diluent or at least one initiator to the oil phase;
(d) heating the oil-in-water emulsion with stirring to a temperature between 25° C. and 100° C. to form the polymeric microcapsule shell by an in-situ oil-in-water reaction of the amine donor(s) with the acceptor component(s); and
(e) obtaining the lipophilic core encapsulated in a polymeric microcapsule shell.

45. The method according to claim 44, wherein the modifying reagent comprises hydrophobic, sterically hindered, charged, or pH responsive functional groups.

46. The method according to claim 44, wherein the modified amine donor comprises residual (unreacted) amine (NH) groups for further reaction and incorporation into a polymeric shell, or into a polymeric shell comprising a poly-ß-amino ester.

47. The method according to claim 46, wherein the modified amine donor comprises at least two residual (unreacted) amine (NH) groups for further reaction and incorporation into a polymeric shell, or into a polymeric shell comprising a poly-ß-amino ester.

48. A method for preparing a microcapsule of claim 1, the method comprising:
(a) preparing an oil-in-water emulsion of (i) an oil phase comprising at least one amine functional donor with at least one acceptor, and at least one lipophilic core; and (ii) a water phase comprising at least one stabilizer, at least one defoamer or at least one emulsifier,
(b) optionally adding at least one catalyst, at least one diluent or at least one initiator to the oil phase;
(c) heating the oil-in-water emulsion with stirring to a temperature between 25° C. and 100° C. and forming the polymeric microcapsule shell by an in-situ oil-in-water reaction of the amine donor(s) with the acceptor component(s);
(e) obtaining the lipophilic core encapsulated in a polymeric microcapsule shell, and
(f) effecting a post-modification reaction on the polymeric shell material to add hydrophobic, sterically hindered, charged, rigid or pH responsive functional groups to the shell material.

49. The method according to claim 48, wherein at least one donor or acceptor reactant comprises a hydroxyl group reactive functionality incorporated into the polymeric shell or polymeric shell comprising poly-ß-amino ester and is used for the subsequent post reactive modification step as in claim 48 (f), to add hydrophobic, sterically hindered, charged, rigid or pH responsive functional groups to the shell material.

50. The method according to claim 49, wherein the hydroxyl functionality is co-introduced via
(i) inclusion of hydroxyethyl acrylate, hydroxyethyl methacrylate, hydroxypropyl acrylate, hydroxypropyl methacrylate, hydroxybutyl acrylate, hydroxybutyl methacrylate, or
(ii) inclusion of hydroxyl functional amines selected from ethanolamine, diethanolamine, amino pentanol, amino butanol, and aminopropanol.

51. The method according to claim 48, wherein the total donor or the total acceptor reactants are used in stoichiometric excess to leave unreacted amine or acceptor functional groups available for subsequent post-reactive modification.

52. The method according to claim 44, wherein the pre-reaction or post modification reaction is carried out to introduce hydrophobic, sterically hindered, rigid, charged or pH responsive functional groups and proceeds via a modifying monofunctional Michael donor or monofunctional Michael acceptor bearing groups reacting with excess or unreacted acceptor or donor functional groups in the other monomeric reactants, or in the polymeric shell.

53. The method according to claim 44, wherein the pre-modification or post-modification reaction is progressed via:
(i) reaction of excess or unreacted amine groups with monofunctional acid, monofunctional acid derivative, difunctional acid, difunctional acid derivative, half ester of difunctional acid, acid chloride, or acid anhydride, or with a monofunctional or difunctional epoxide, isocyanate, halocarbon or aldehyde or ketone; or
(ii) reaction of an available or added hydroxyl group with monofunctional acid, difunctional acid, half ester of difunctional acid, acid chloride, acid anhydride, and derivatives thereof or with a monofunctional or difunctional epoxide, halocarbon, isocyanate, aldehyde or ketone; or via,
(v) reaction with an amino acid.

54. The method according to claim 53 (i) or (ii), wherein the acid or acid derivatives are selected from the group consisting of C2-C12 alkanoic acids, oxalic acid, propanedioic acid, butanedioic acid, hexanedioic acid, octanedioic acid, decanedioic acid, sebacic acid, dodecanedioic acid, dodecenylsuccinic acid, octenylsuccinic acid, cholesteric acid, itaconic acid, maleic acid, fumaric acid, and malonic acid.

55. The method according to claim 44, wherein the pre-modification or post-modification reaction is via a Michael Addition Reaction of a selected donor and acceptor combination.

56. The method according to claim 45, wherein the functional reactant is an acceptor selected from the group consisting of:
(i) acrylamido sulfonic acid (AMPS), acrylic acid, methacrylic acid, carboxyethyl acrylate, dimethylaminoethyl acrylate or methacrylate (DMAEA/DMAEMA), tert-butyl amino ethyl acrylate or methacrylate (TBAEA/TBAEMA),
(ii) cationically charged mono functional acrylate or methacrylate selected from trimethyl aminoethyl methacrylate chloride ((MBJ), 3-Acrylamidopropyl) trimethyl ammonium chloride (APTAC), 3-methacrylamido propyl trimethyl ammonium chloride (MAPTAC), trimethylamine ethyl acrylate chloride (MBS),
(iii) an acryloyl- or methacryloyl-phosphate,
(iv) lauryl, n-alkyl or branched alkyl acrylate or methacrylate, sec-alkyl acrylate or methacrylate, tert-butyl acrylate or methacrylate, tert-alkyl acrylate or methacrylate, isobornyl acrylate or methacrylate, menthyl acrylate or methacrylate, cyclohexyl acrylate or methacrylate or any other cycloaliphatic acrylate or methacrylate, or
(v) phenoxyethyl-, benzyl-, or any phenyl ring containing mono acrylate or mono methacrylate.

57. The method according to claim 44, wherein the pre-modification or post-modification reaction is a Michael Addition Reaction of a methacrylate acceptor(s) and wherein, the shell formation reaction is a Michael Addition of an acrylate acceptor(s); and
wherein, the final shell material comprises both ß-aminoethyl ester groups, and ß-amino-(1-methyl-ethyl) ester groups.

58. The method according to claim 41, wherein zein, a protein, a polypeptide, or a polymer having hydrophobic, sterically hindered, charged or pH responsive functional groups are added prior to, during or at the end of the reaction.

59. The method according to claim 58, wherein the zein, protein, polypeptide, or a polymer having hydrophobic, sterically hindered, charged or pH responsive functional groups is dissolved in the oil phase, or in an oil phase component, prior to the shell formation reaction step.

60. The method according to claim 58, wherein the zein, protein, polypeptide, or a polymer having hydrophobic, sterically hindered, charged or pH responsive functional groups is present as a solid powder or dispersion.

61. The method according to claim 41, wherein the polymer is selected from xanthan gum, guar gum, cationic guar, polysaccharide, or combinations thereof and are added prior to, during or at the end of the reaction.

62. The method according to claim 41, wherein the hydrophobic or sterically hindered groups are selected from the group consisting of methyl, ethyl, propyl, butyl, C5-C20 alkyl, C5-C20 branched alkyl, tertiary methyl, tertiary ethyl, tertiary propyl, tertiary butyl, cyclohexyl, alkyl-cyclohexyl, isobornyl, norbornyl, menthyl, cholesteryl, cycloaliphatic, phenyl, phenoxyethyl, benzyl, and aryl moieties.

63. The method according to claim 41, wherein the charged or pH responsive functional group is selected from the group consisting of carboxylic acid, carboxylate, sulfonic acid, sulfonate, phosphonic acid, phosphate, boronic acid, borate, quaternary ammonium, ammonium, tertiary amine, secondary amine, primary amine, amine containing heterocyclic bases, amino-acids, salts, and conjugates thereof.

64. The method according to claim 41, wherein the polymeric shell comprises a crosslinked poly ß-amino-ester polymer.

65. The method according to claim 40, wherein the water phase or oil phase, or where both phases, comprise a radical initiator system selected from peroxide based, an azo based, a redox based system, or comprises a radical chain transfer agent, added prior to, during or at the end of the process.

66. The method according to claim 40, wherein the process incorporates a radical addition or polymerization reaction to add crosslinking or to consume residual acceptor or donor functionality, wherein such reaction is performed at a temperature of ≤130° C. or ≤100° C.

67. The method according to claim 40, wherein the method further comprises crosslinking reaction performed at a temperature of ≤130° C. or at a temperature of ≤100° C. or of ≤30° C. using glutaraldehyde glyoxal, isocyanates, acid, acid derivatives, or epoxides.

68. The method according to claim 40, wherein the oil phase optionally comprises at least one diluent or solvent.

69. The method according to claim 68, wherein the diluent or solvent is selected from the group consisting of hydrocarbon oil, alkanes, an ester oils, a fatty acid esters, an aliphatic esters, and alkylene carbonates.

70. The method according to claim 40, wherein the oil phase is homogeneous and is prepared with optional heating up to a temperature of 100° C.

71. The method according to claim 40, wherein the water phase optionally comprises at least one additive selected from the group consisting of surfactants, defoamers, rheology modifiers, thickeners, partitioning inhibitors, radical inhibitors, catalysts, radical initiators or combinations thereof.

72. The method according to claim 40, wherein the
stabilizer or emulsifier is selected from the group consisting of polyvinyl alcohol, hydroxyethyl cellulose, guar, cationic guar, xanthan gum, polysaccharides, polyvinylpyrrolidone and combinations thereof, and
defoamer is selected from the group consisting of liquid hydrocarbons, oils, hydrophobic silicas, fatty acids, alkoxylated compounds, polyethers, polyalkylene glycols, and nonionic emulsifiers.

73. The method according to claim 40, wherein the oil in water emulsion is prepared with or without the application of heat.

74. The method according to claim 40, wherein the at least one donor component and the at least one acceptor component each have a reactive functionality of at least two.

75. The method according to claim 40, wherein the at least one amine donor and the at least one each have a reactive functionality of at least three.

76. The method according to claim 40, wherein the amine is a difunctional primary amine, a difunctional secondary amine, a multifunctional primary amine, a multifunctional primary or a multifunctional secondary amine.

77. The method according to claim 40, wherein the amine component comprises a C₂-C₂₀ aliphatic chain, C4-C7 cyclic or a C4-C7 heterocyclic ring.

78. The method according to claim 40, wherein the amine component is selected from the group consisting of C2-C16 alkylamine or primary cyclo-alkylamine, 4,4′trimethylenepiperidine (TMPP), isophorone diamine (IPD), bis-(aminomethyl) cyclohexane, cyclohexane diamine, piperazine, aminoethylpiperazine, bis-amino-norbornane, ethylene diamine, diethylene triamine, diethylene diamine, triethylene tetramine, tetraethylene pentamine, pentaethylene hexamine (PEHA), spermine, tris(2-aminoethyl)amine, bis(3-aminopropyl)amine, hexamethylenediamine (HMDA), diamino-propane, diamino-butane, diamino-pentane, diamino-octane, decane diamine, dodecane diamine, amino-ethanol, amino-propanol, amino-butanol, amino pentanol, any polyfunctional amine, and polyethyleneimine or any derivatives of such di- or multi-functional amines with available amine groups for reaction with acceptors.

79. The method according to claim 40, wherein the at least one Michael acceptor is selected from the group consisting of
(a) itaconate containing polyester,
(b) an acrylate, diacrylate, or multifunctional acrylate of a polyester;
(c) an acrylate, diacrylate, or multifunctional acrylate of an epoxide;
(d) an acrylate, diacrylate, or multifunctional acrylate of a urethane;
(e) an acrylate, diacrylate, or multifunctional acrylate of a polyether or a diol or polyol;
(f) an acrylate, diacrylate, or multifunctional acrylate of an amine;
(g) methacrylate analogues of (a) to (f) components,
and combinations thereof.

80. The method according to claim 40, wherein the in-situ reaction to form the polymeric shell is an addition reaction at a temperature of ≤80° C., or ≤60° C., or ≤50° C.

81. The method according to claim 40, wherein the acceptor is selected from the group consisting of multifunctional acrylate, methacrylate, acrylamide, methacrylamide, maleate, fumarate, itaconate, malonate, crotonate, citraconate, maleimide and mixtures thereof.

82. The method according to claim 40, wherein the catalyst is a tertiary amine.

83. The method according to claim 40, wherein the method further comprises adding a coating or outer layer to the microcapsule.

84. The method according to claim 83, wherein the coating or outer layer is made from at least one polymer selected from the group consisting of xanthan gum, guar, cationic guar, polysaccharide gum, an alginate polymer, hydroxyethyl cellulose, carboxymethylcellulose, zein protein, a hydrogel, a coacervate, a polymer bearing hydrophobic, charged, pH responsive groups, and combinations thereof.

85. The method according to claim 40, wherein the microcapsule slurry is spray dried, freeze dried, or dried via a fluid bed process to isolate dry microcapsules.

86. The method according to claim 83 wherein the coating or outer layer comprises octentyl succinate modified starch, dodecenyl succinate modified starch, or tannic acid.
